(19) United States
(12) Patent Application Publication
(10) Pub. No.: US 2024/0350433 A1
(43) Pub. Date: Oct. 24, 2024

(54) TREATMENT COMPOSITIONS AND METHODS

(71) Applicants: Yale University, New Haven, CT (US); THE UNITED STATES GOVERNMENT AS REPRESENTED BY THE DEPARTMENT OF VETERANS AFFAIRS, Washington, DC (US)

(72) Inventors: John H. Krystal, New Haven, CT (US); Gihyun Yoon, New Haven, CT (US); Ismene L. Petrakis, New Haven, CT (US)

(21) Appl. No.: 18/684,563

(22) PCT Filed: Aug. 16, 2022
(86) PCT No.: PCT/US2022/040441

§ 371 Date: Feb. 16, 2024

Related U.S. Application Data
(60) Provisional application No. 63/234,088, filed on Aug. 17, 2021.

(51) Int. Cl. A61K 31/135; A61K 31/485; A61P 25/24; A61P 25/32

(52) U.S. Cl. CPC A61K 31/135; A61K 31/485; A61P 25/24; A61P 25/32

(57) ABSTRACT

The present application provides pharmaceutical compositions and methods for treating diseases or disorders. The pharmaceutical composition comprises N-methyl-D-aspartate receptor modulator and μ-opioid receptor modulator. The present application also discloses formulations, dosing and administration routes for the pharmaceutical composition. Diseases can be treated by the pharmaceutical composition are also described.

### CROSS-REFERENCE TO RELATED APPLICATIONS

This application claims priority to U.S. Provisional Application No. 63/234,088 filed Aug. 17, 2021, which is incorporated herein by reference in the entirety for all purposes.

### BACKGROUND

The N-methyl-D-aspartate (“NMDA”) glutamate receptor (also known as “NMDAR” or “NMDA-R”) is an ionotropic glutamate receptor found in nerve cells. The NMDA receptor is activated in a depolarized membrane when glutamate and glycine or D-serine bind to it. That activation opens a channel in the receptor that enables cations, particularly calcium ions, to enter the neuron, triggering neuronal excitation and initiating many forms of neuroplasticity. As a result, the NMDA receptor plays important roles in initiating and coordinating neural circuit activity as well as playing roles in neurodevelopment, learning and memory, neurotoxicity, and other functions.

Modulators of the NMDA receptor are under development for the treatment of various mood disorders. However, NMDA receptor modulators are plagued with high abuse potential, and drug supplies are often misused as recreational drugs (Schatzberg, A. F. (2019). A Word to the Wise About Intranasal Esketamine. Am J Psychiatry, 176, 422-424).

There is thus a need in the art for novel compositions and methods that allow for the administration of NMDA receptor modulators to subjects who can benefit from their pharmacological effects, while minimizing or eliminating abuse liability for these modulators. The present application addresses and meets this need.

### BRIEF SUMMARY

The present disclosure provides a pharmaceutical composition comprising a N-methyl-D-aspartate receptor modulator and a μ-opioid receptor modulator. In certain embodiments, the μ-opioid receptor modulator is present in an amount that, when administered to a subject, occupies at least 10% of the subject's μ-opioid receptors in vivo as measured by positron emission tomography with a [¹¹C]-carfentanil ligand (wherein carfentanyl corresponds to methyl 1-(2-phenylethyl)-4-[phenyl(propanoyl)amino]piperidine-4-carboxylate, or a salt and/or solvate thereof).

The present disclosure further provides a method of treating, ameliorating, and/or preventing a disease or disorder in a subject in need thereof. In certain embodiments, the method comprises administering to the subject at least one of the pharmaceutical compositions contemplated herein. In certain embodiments, the method comprises administering to the subject a N-methyl-D-aspartate receptor modulator and a μ-opioid receptor modulator. In certain embodiments, the μ-opioid receptor modulator is administered in an amount that occupies at least 10% of the subject's μ-opioid receptors in vivo as measured by positron emission tomography with a [¹¹C]-carfentanil ligand.

Also provided herein is a method of treating, ameliorating, and/or preventing a disease or disorder in a subject in need thereof comprising administering a coformulation to the subject. In some embodiments, the coformulation comprises a N-methyl-D-aspartate receptor modulator and a μ-opioid receptor modulator. In some embodiments, the coformulation is administered to the subject repeatedly.

In some embodiments, the μ-opioid receptor modulator is present in an amount that, when administered to a subject, occupies at least 25% of the subject's μ-opioid receptors in vivo. In some embodiments, the μ-opioid receptor modulator is present in an amount that, when administered to a subject, occupies at least 50% of the subject's μ-opioid receptors in vivo. In some embodiments, the μ-opioid receptor modulator is present in an amount that, when administered to a subject, occupies at least 75% of the subject's μ-opioid receptors in vivo. In some embodiments, the μ-opioid receptor modulator is present in an amount that, when administered to a subject, occupies at least 90% of the subject's μ-opioid receptors in vivo. In some embodiments, the μ-opioid receptor modulator is present in an amount that, when administered to a subject, occupies at least 99% of the subject's μ-opioid receptors in vivo.

In some embodiments, the μ-opioid receptor modulator is administered to the subject in an amount that occupies at least 25% of the subject's μ-opioid receptors in vivo. In some embodiments, the μ-opioid receptor modulator is administered to the subject in an amount that occupies at least 50% of the subject's μ-opioid receptors in vivo. In some embodiments, the μ-opioid receptor modulator is administered to the subject in an amount that occupies at least 75% of the subject's μ-opioid receptors in vivo. In some embodiments, the μ-opioid receptor modulator is administered to the subject in an amount that occupies at least 90% of the subject's μ-opioid receptors in vivo. In some embodiments, the μ-opioid receptor modulator is administered to the subject in an amount that occupies at least 99% of the subject's μ-opioid receptors in vivo.

In some embodiments, the N-methyl-D-aspartate receptor modulator is selected from the group consisting of a N-methyl-D-aspartate receptor antagonist, a N-methyl-D-aspartate receptor negative allosteric modulator, and a N-methyl-D-aspartate receptor partial agonist.

In some embodiments, the N-methyl-D-aspartate receptor modulator is a N-methyl-D-aspartate receptor antagonist. In some embodiments, the N-methyl-D-aspartate receptor modulator is a N-methyl-D-aspartate receptor negative allosteric modulator. In some embodiments, the N-methyl-D-aspartate receptor modulator is a N-methyl-D-aspartate receptor partial agonist.

In some embodiments, the N-methyl-D-aspartate receptor modulator comprises a compound selected from the group consisting of ketamine, R-ketamine, S-ketamine, nitrous oxide, memantine, amantadine, racemic dextromethorphan, dextromethorphan, lanicemine, phencyclidine, dizocilpine, CERC-301, CGP 37849, 1-aminocylopropanecarboxylic acid, traxoprodil, Ro 25-6981, eliprodil, methoxetamine, CPPene, AP5, AP7, Selfotel (CGS-19755), minocycline, nitromemantine, PD-137889, rolicyclidine, tenocyclidine, methoxydine, tiletamine, neramexane, etoxadrol, dexoxadrol, WMS-2539, NEFA, remacemide, 3-MeO-PCP, 8A-PDHQ, atomoxetine, AZD6765, agmatine, chloroform, delucemine, dextrallorphan, dextrorphan, diphenidine, eticyclidine, gacyclidine, aptiganel, HU-211, huperzine A, dipeptide D-Phe-L-Tyr, ibogaine, rhynchophylline, rapastinel, NRX-1074, 7-Chlorokynurenic acid, 4-Chlorokynurenine, 5,7-Dichlorokynurenic acid, Kynurenic acid, TK-40, L-Phenylalanine, xenon, methadone, EU1180-438, radiprodil, Ifenprodil, TCN-201, MPX-004, MPX-007, NAB-14, EVT-101, QNZ-46, DQP-1105, pregnanolone sulfate (3α5βS), UBP608, UBP618, UBP551, UBP512, HA-966, felbamate, PEAQX (NVP-AAM077), PD0196860, RGH896, MK0657, L701324, LY293558, LY300164, LY246492, LY202157, NYX-783, and/or any salt, solvate, enantiomer, tautomer, stereoisomer, and/or geometric isomer thereof, and/or any mixtures thereof.

In some embodiments, the N-methyl-D-aspartate receptor modulator comprises ketamine. In some embodiments, the N-methyl-D-aspartate receptor modulator comprises R-ketamine. In some embodiments, the N-methyl-D-aspartate receptor modulator comprises S-ketamine. In some embodiments, the N-methyl-D-aspartate receptor modulator comprises nitrous oxide. In some embodiments, the N-methyl-D-aspartate receptor modulator comprises memantine. In some embodiments, the N-methyl-D-aspartate receptor modulator comprises amantadine. In some embodiments, the N-methyl-D-aspartate receptor modulator comprises racemic dextromethorphan. In some embodiments, the N-methyl-D-aspartate receptor modulator comprises dextromethorphan. In some embodiments, the N-methyl-D-aspartate receptor modulator comprises lanicemine. In some embodiments, the N-methyl-D-aspartate receptor modulator comprises phencyclidine. In some embodiments, the N-methyl-D-aspartate receptor modulator comprises dizocilpine. In some embodiments, the N-methyl-D-aspartate receptor modulator comprises CERC-301. In some embodiments, the N-methyl-D-aspartate receptor modulator comprises CGP 37849. In some embodiments, the N-methyl-D-aspartate receptor modulator comprises 1-aminocylopropanecarboxylic acid. In some embodiments, the N-methyl-D-aspartate receptor modulator comprises traxoprodil. In some embodiments, the N-methyl-D-aspartate receptor modulator comprises Ro 25-6981. In some embodiments, the N-methyl-D-aspartate receptor modulator comprises eliprodil. In some embodiments, the N-methyl-D-aspartate receptor modulator comprises methoxetamine. In some embodiments, the N-methyl-D-aspartate receptor modulator comprises CPPene. In some embodiments, the N-methyl-D-aspartate receptor modulator comprises AP5. In some embodiments, the N-methyl-D-aspartate receptor modulator comprises AP7. In some embodiments, the N-methyl-D-aspartate receptor modulator comprises Selfotel (CGS-19755). In some embodiments, the N-methyl-D-aspartate receptor modulator comprises minocycline. In some embodiments, the N-methyl-D-aspartate receptor modulator comprises nitromemantine. In some embodiments, the N-methyl-D-aspartate receptor modulator comprises PD-137889. In some embodiments, the N-methyl-D-aspartate receptor modulator comprises rolicyclidine. In some embodiments, the N-methyl-D-aspartate receptor modulator comprises tenocyclidine. In some embodiments, the N-methyl-D-aspartate receptor modulator comprises methoxydine. In some embodiments, the N-methyl-D-aspartate receptor modulator comprises tiletamine. In some embodiments, the N-methyl-D-aspartate receptor modulator comprises neramexane. In some embodiments, the N-methyl-D-aspartate receptor modulator comprises etoxadrol. In some embodiments, the N-methyl-D-aspartate receptor modulator comprises dexoxadrol. In some embodiments, the N-methyl-D-aspartate receptor modulator comprises WMS-2539. In some embodiments, the N-methyl-D-aspartate receptor modulator comprises NEFA. In some embodiments, the N-methyl-D-aspartate receptor modulator comprises remacemide. In some embodiments, the N-methyl-D-aspartate receptor modulator comprises 3-MeO-PCP. In some embodiments, the N-methyl-D-aspartate receptor modulator comprises 8A-PDHQ. In some embodiments, the N-methyl-D-aspartate receptor modulator comprises atomoxetine. In some embodiments, the N-methyl-D-aspartate receptor modulator comprises AZD6765. In some embodiments, the N-methyl-D-aspartate receptor modulator comprises agmatine. In some embodiments, the N-methyl-D-aspartate receptor modulator comprises chloroform. In some embodiments, the N-methyl-D-aspartate receptor modulator comprises delucemine. In some embodiments, the N-methyl-D-aspartate receptor modulator comprises dextrallorphan. In some embodiments, the N-methyl-D-aspartate receptor modulator comprises dextrorphan. In some embodiments, the N-methyl-D-aspartate receptor modulator comprises diphenidine. In some embodiments, the N-methyl-D-aspartate receptor modulator comprises eticyclidine. In some embodiments, the N-methyl-D-aspartate receptor modulator comprises gacyclidine. In some embodiments, the N-methyl-D-aspartate receptor modulator comprises aptiganel. In some embodiments, the N-methyl-D-aspartate receptor modulator comprises HU-211. In some embodiments, the N-methyl-D-aspartate receptor modulator comprises huperzine A. In some embodiments, the N-methyl-D-aspartate receptor modulator comprises dipeptide D-Phe-L-Tyr. In some embodiments, the N-methyl-D-aspartate receptor modulator comprises ibogaine. In some embodiments, the N-methyl-D-aspartate receptor modulator comprises rhynchophylline. In some embodiments, the N-methyl-D-aspartate receptor modulator comprises rapastinel. In some embodiments, the N-methyl-D-aspartate receptor modulator comprises NRX-1074. In some embodiments, the N-methyl-D-aspartate receptor modulator comprises 7-Chlorokynurenic acid. In some embodiments, the N-methyl-D-aspartate receptor modulator comprises 4-Chlorokynurenine. In some embodiments, the N-methyl-D-aspartate receptor modulator comprises 5,7-Dichlorokynurenic acid. In some embodiments, the N-methyl-D-aspartate receptor modulator comprises Kynurenic acid. In some embodiments, the N-methyl-D-aspartate receptor modulator comprises TK-40. In some embodiments, the N-methyl-D-aspartate receptor modulator comprises L-Phenylalanine. In some embodiments, the N-methyl-D-aspartate receptor modulator comprises xenon. In some embodiments, the N-methyl-D-aspartate receptor modulator comprises methadone. In some embodiments, the N-methyl-D-aspartate receptor modulator comprises EU1180-438. In some embodiments, the N-methyl-D-aspartate receptor modulator comprises radiprodil. In some embodiments, the N-methyl-D-aspartate receptor modulator comprises Ifenprodil. In some embodiments, the N-methyl-D-aspartate receptor modulator comprises TCN-201. In some embodiments, the N-methyl-D-aspartate receptor modulator comprises MPX-004. In some embodiments, the N-methyl-D-aspartate receptor modulator comprises MPX-007. In some embodiments, the N-methyl-D-aspartate receptor modulator comprises NAB-14. In some embodiments, the N-methyl-D-aspartate receptor modulator comprises EVT-101. In some embodiments, the N-methyl-D-aspartate receptor modulator comprises QNZ-46. In some embodiments, the N-methyl-D-aspartate receptor modulator comprises DQP-1105. In some embodiments, the N-methyl-D-aspartate receptor modulator comprises pregnanolone sulfate (3α5βS). In some embodiments, the N-methyl-D-aspartate receptor modulator comprises UBP608. In some embodiments, the N-methyl-D-aspartate receptor modulator comprises UBP618. In some embodiments, the N-methyl-D-aspartate receptor modulator comprises UBP551. In some embodiments, the N-methyl-D-aspartate receptor modulator comprises UBP512. In some embodiments, the N-methyl-D-aspartate receptor modulator comprises HA-966. In some embodiments, the N-methyl-D-aspartate receptor modulator comprises felbamate. In some embodiments, the N-methyl-D-aspartate receptor modulator comprises PEAQX (NVP-AAM077). In some embodiments, the N-methyl-D-aspartate receptor modulator comprises PD0196860. In some embodiments, the N-methyl-D-aspartate receptor modulator comprises RGH896. In some embodiments, the N-methyl-D-aspartate receptor modulator comprises MK0657. In some embodiments, the N-methyl-D-aspartate receptor modulator comprises L701324. In some embodiments, the N-methyl-D-aspartate receptor modulator comprises LY293558. In some embodiments, the N-methyl-D-aspartate receptor modulator comprises LY300164. In some embodiments, the N-methyl-D-aspartate receptor modulator comprises LY246492. In some embodiments, the N-methyl-D-aspartate receptor modulator comprises LY202157. In some embodiments, the N-methyl-D-aspartate receptor modulator comprises NYX-783. In some embodiments, the N-methyl-D-aspartate receptor modulator comprises any N-methyl-D-aspartate receptor modulator recited herein and/or any salt, solvate, enantiomer, tautomer, stereoisomer, and/or geometric isomer thereof, and/or any mixtures thereof.

In some embodiments, the N-methyl-D-aspartate receptor modulator comprises ketamine, and/or any salt, solvate, enantiomer, tautomer, stereoisomer, and/or geometric isomer thereof, and/or any mixtures thereof.

In some embodiments, the N-methyl-D-aspartate receptor modulator is present in an amount of about 10 mg to about 60 mg. In some embodiments, the N-methyl-D-aspartate receptor modulator is present in an amount of about 15 mg to about 95 mg. In some embodiments, the N-methyl-D-aspartate receptor modulator is present in an amount of about 50 mg.

In some embodiments, the N-methyl-D-aspartate receptor modulator is administered in an amount of about 10 mg to about 60 mg. In some embodiments, the N-methyl-D-aspartate receptor modulator is administered in an amount of about 15 mg to about 95 mg. In some embodiments, the N-methyl-D-aspartate receptor modulator is administered in an amount of about 50 mg.

In some embodiments, the μ-opioid receptor modulator is selected from the group consisting of a μ-opioid receptor antagonist, a μ-opioid receptor negative allosteric modulator, and a μ-opioid receptor partial agonist.

In some embodiments, the μ-opioid receptor modulator is a μ-opioid receptor antagonist. In some embodiments, the μ-opioid receptor modulator is a μ-opioid receptor negative allosteric modulator. In some embodiments, the μ-opioid receptor modulator is a μ-opioid receptor partial agonist.

In some embodiments, the μ-opioid receptor modulator comprises a compound selected from the group consisting of naltrexone, naloxone, nalmefene, nalodeine, and/or any salt, solvate, enantiomer, tautomer, stereoisomer, and/or geometric isomer thereof, and/or any mixtures thereof. In some embodiments, the μ-opioid receptor modulator comprises naltrexone, and/or any salt, solvate, enantiomer, tautomer, stereoisomer, and/or geometric isomer thereof, and/or any mixtures thereof.

In some embodiments, the μ-opioid receptor modulator comprises naltrexone. In some embodiments, the μ-opioid receptor modulator comprises naloxone. In some embodiments, the μ-opioid receptor modulator comprises nalmefene. In some embodiments, the μ-opioid receptor modulator comprises nalodeine. In some embodiments, the μ-opioid receptor modulator comprises any μ-opioid receptor modulator recited herein, and/or any salt, solvate, enantiomer, tautomer, stereoisomer, and/or geometric isomer thereof, and/or any mixtures thereof.

In some embodiments, the naltrexone is formulated for extended-release (long-acting naltrexone) which prolongs the serum half-life of the naltrexone. In some embodiments, the long-acting naltrexone is VIVITROL®. In some embodiments, VIVITROL® is an injectable suspension containing 380 mg of naltrexone in a microsphere formulation and 4 mL diluent. In some embodiments, in VIVITROL® naltrexone is incorporated in 75:25 polylactide-co-glycolide (PLG) at a concentration of 337 mg of naltrexone per gram of microspheres. In some embodiments, the composition of the diluent in VIVITROL® includes carboxymethylcellulose sodium salt, polysorbate 20, sodium chloride, and water for injection. In some embodiments, the naloxone is formulated for extended-release (long-acting naloxone) which prolongs the serum half-life of the naloxone. In some embodiments, the nalmefene is formulated for extended-release (long-acting nalmefene) which prolongs the serum half-life of the nalmefene. In some embodiments, the μ-opioid receptor modulator comprises naltrexone, and/or any salt, solvate, enantiomer, tautomer, stereoisomer, and/or geometric isomer thereof, and/or any mixtures thereof.

In some embodiments, the μ-opioid receptor modulator is present in an amount of lower than about 400 mg. In some embodiments, the μ-opioid receptor modulator is present in an amount of about 300 mg to about 400 mg. In some embodiments, the μ-opioid receptor modulator is present in an amount of about 200 mg to about 300 mg. In some embodiments, the μ-opioid receptor modulator is present in an amount of about 100 mg to about 200 mg. In some embodiments, the μ-opioid receptor modulator is present in an amount of about 5 mg to about 100 mg. In some embodiments, the μ-opioid receptor modulator is present in an amount of about 25 mg to about 100 mg. In some embodiments, the μ-opioid receptor modulator is present in an amount of about 1 mg to about 90 mg. In some embodiments, the μ-opioid receptor modulator is present in an amount of about 2 mg to about 10 mg.

In some embodiments, the μ-opioid receptor modulator is administered in an amount of lower than about 400 mg. In some embodiments, the μ-opioid receptor modulator is administered in an amount of about 300 mg to about 400 mg. In some embodiments, the μ-opioid receptor modulator is administered in an amount of about 200 mg to about 300 mg. In some embodiments, the μ-opioid receptor modulator is administered in an amount of about 100 mg to about 200 mg. In some embodiments, the μ-opioid receptor modulator is administered in an amount of about 5 mg to about 100 mg. In some embodiments, the μ-opioid receptor modulator is administered in an amount of about 25 mg to about 100 mg. In some embodiments, the μ-opioid receptor modulator is administered in an amount of about 1 mg to about 90 mg. In some embodiments, the μ-opioid receptor modulator is administered in an amount of about 2 mg to about 10 mg.

In some embodiments, the N-methyl-D-aspartate receptor modulator comprises ketamine, and/or any salt, solvate, enantiomer, tautomer, stereoisomer, and/or geometric isomer thereof, and/or any mixtures thereof; and the μ-opioid receptor modulator comprises naltrexone, and/or any salt, solvate, enantiomer, tautomer, stereoisomer, and/or geometric isomer thereof, and/or any mixtures thereof. In some embodiments, the N-methyl-D-aspartate receptor modulator is present in an amount of about 10 mg to about 95 mg, and the μ-opioid receptor modulator is present in an amount of about 10 mg to about 400 mg. In some embodiments, the N-methyl-D-aspartate receptor modulator is administered in an amount of about 10 mg to about 95 mg, and the μ-opioid receptor modulator is administered in an amount of about 10 mg to about 400 mg.

In some embodiments, the N-methyl-D-aspartate receptor modulator and the μ-opioid receptor modulator are administered concurrently. In some embodiments, the N-methyl-D-aspartate receptor modulator and the μ-opioid receptor modulator are administered separately. In some embodiments, the N-methyl-D-aspartate receptor modulator and the μ-opioid receptor modulator are administered sequentially. In some embodiments, the N-methyl-D-aspartate receptor modulator is administered once per day. In some embodiments, the N-methyl-D-aspartate receptor modulator is administered twice per day. In some embodiments, the N-methyl-D-aspartate receptor modulator is administered three times per day. In some embodiments, the N-methyl-D-aspartate receptor modulator is administered once per week. In some embodiments, the N-methyl-D-aspartate receptor modulator is administered twice per week. In some embodiments, the N-methyl-D-aspartate receptor modulator is administered three times per week. In some embodiments, the N-methyl-D-aspartate receptor modulator is administered once per month. In some embodiments, the N-methyl-D-aspartate receptor modulator is administered twice per month. In some embodiments, the N-methyl-D-aspartate receptor modulator is administered three times per month. In some embodiments, the μ-opioid receptor modulator is administered once per day. In some embodiments, the μ-opioid receptor modulator is administered twice per day. In some embodiments, the μ-opioid receptor modulator is administered three times per day. In some embodiments, the μ-opioid receptor modulator is administered once per week. In some embodiments, the μ-opioid receptor modulator is administered twice per week. In some embodiments, the μ-opioid receptor modulator is administered three times per week. In some embodiments, the μ-opioid receptor modulator is administered once per month. In some embodiments, the μ-opioid receptor modulator is administered twice per month. In some embodiments, the μ-opioid receptor modulator is administered three times per month. In some embodiments, the N-methyl-D-aspartate receptor modulator is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous. In some embodiments, the μ-opioid receptor modulator is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

In some embodiments, the disease is at least one selected from the group consisting of major depressive disorder, major depressive episode in bipolar disorder (bipolar depression), bipolar I disorder, bipolar II disorder, persistent depressive disorder (dysthymia), disruptive mood dysregulation disorder, major depressive disorder (including major depressive episode), premenstrual dysphoric disorder, substance/medication-induced depressive disorder, depressive disorder due to another medical condition, other specified depressive disorder, unspecified depressive disorder, anxiety disorder, generalized anxiety disorder, social anxiety disorder (social phobia), specific phobia, panic disorder, agoraphobia, separation anxiety disorder, selective mutism, substance-induced anxiety disorder, medication-induced anxiety disorder, anxiety disorder due to another medical condition, borderline personality disorder, treatment-resistant depression, unspecified anxiety disorder, and posttraumatic stress disorder. In some embodiments, the subject suffers from a comorbid substance use disorder.

In some embodiments, the disease or disorder is major depressive disorder. In some embodiments, the disease or disorder is major depressive episode in bipolar disorder (bipolar depression). In some embodiments, the disease or disorder is bipolar I disorder. In some embodiments, the disease or disorder is bipolar II disorder. In some embodiments, the disease or disorder is persistent depressive disorder (dysthymia). In some embodiments, the disease or disorder is disruptive mood dysregulation disorder. In some embodiments, the disease or disorder is major depressive disorder (including major depressive episode). In some embodiments, the disease or disorder is premenstrual dysphoric disorder. In some embodiments, the disease or disorder is substance/medication-induced depressive disorder. In some embodiments, the disease or disorder is depressive disorder due to another medical condition. In some embodiments, the disease or disorder is other specified depressive disorder. In some embodiments, the disease or disorder is unspecified depressive disorder. In some embodiments, the disease or disorder is anxiety disorder. In some embodiments, the disease or disorder is generalized anxiety disorder. In some embodiments, the disease or disorder is social anxiety disorder (social phobia). In some embodiments, the disease or disorder is specific phobia. In some embodiments, the disease or disorder is panic disorder. In some embodiments, the disease or disorder is agoraphobia. In some embodiments, the disease or disorder is separation anxiety disorder. In some embodiments, the disease or disorder is selective mutism. In some embodiments, the disease or disorder is substance-induced anxiety disorder. In some embodiments, the disease or disorder is medication-induced anxiety disorder. In some embodiments, the disease or disorder is anxiety disorder due to another medical condition. In some embodiments, the disease or disorder is borderline personality disorder. In some embodiments, the disease or disorder is treatment-resistant depression. In some embodiments, the disease or disorder is unspecified anxiety disorder. In some embodiments, the disease or disorder is posttraumatic stress disorder. In some embodiments, the subject suffers from a comorbid substance use disorder.

In some embodiments, the coformulation is administered once per day, twice per day, or three times per day. In some embodiments, the coformulation is administered once per week, twice per week, or three times per week. In some embodiments, the coformulation is administered once per month, twice per month, or three times per month. In some embodiments, the coformulation is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

In some embodiments, presence of the μ-opioid receptor modulator prevents, ameliorates, and/or minimizes abuse of the N-methyl-D-aspartate receptor modulator and an abused substance that is not the N-methyl-D-aspartate receptor modulator. In some embodiments, administration of the μ-opioid receptor modulator prevents, ameliorates, and/or minimizes abuse of the N-methyl-D-aspartate receptor modulator and an abused substance that is not the N-methyl-D-aspartate receptor modulator.

In some embodiments, the abused substance is selected from the group consisting of alcohol, a stimulant, an opioid, cannabis, a hallucinogen, an inhalant, a sedative, a hypnotic, an anxiolytic, tobacco, caffeine, nicotine, and other (unknown) substances. In some embodiments, the stimulant comprises cocaine and/or amphetamine. In some embodiments, the hallucinogen comprises lysergic acid diethylamide (LSD) and/or phencyclidine. In some embodiments, the anxiolytic comprises a barbiturate and/or a benzodiazepine. In some embodiments, the administering has at least one effect selected from the group consisting of reduced anxiety, reduced irritability, reduced anger, and reduced alcohol consumption.

In some embodiments, the abused substance is alcohol. In some embodiments, the abused substance is a stimulant. In some embodiments, the abused substance is an opioid. In some embodiments, the abused substance is cannabis. In some embodiments, the abused substance is a hallucinogen. In some embodiments, the abused substance is an inhalant. In some embodiments, the abused substance is a sedative. In some embodiments, the abused substance is a hypnotic. In some embodiments, the abused substance is an anxiolytic. In some embodiments, the abused substance is tobacco. In some embodiments, the abused substance is caffeine. In some embodiments, the abused substance is nicotine. In some embodiments, the abused substance is other (unknown) substances. In some embodiments, the stimulant comprises cocaine. In some embodiments, the stimulant comprises amphetamine. In some embodiments, the hallucinogen comprises lysergic acid diethylamide (LSD). In some embodiments, the hallucinogen comprises phencyclidine. In some embodiments, the anxiolytic comprises a barbiturate. In some embodiments, the anxiolytic comprises benzodiazepine. In some embodiments, the administering reduces anxiety. In some embodiments, the administering reduces irritability. In some embodiments, the administering reduces anger. In some embodiments, the administering reduces alcohol consumption.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 shows a percentage of responders on Montgomery-Asberg Depression Rating Scale (MADRS) per visit in accordance with aspects of the present disclosure.

FIG. 2 shows a continuous score on MADRS per visit in accordance with aspects of the present disclosure.

FIG. 3 shows a changing means and standard deviation per visit in accordance with aspects of the present disclosure.

FIG. 4 shows a percentage of remission (MADRS<10) per visit in accordance with aspects of the present disclosure.

FIG. 5 shows a mean value of DAPS drug liking per visit in accordance with aspects of the present disclosure.

FIG. 6 shows a mean value of Yale Craving Scale (YCS) at 60 minutes before an infusion per visit in accordance with aspects of the present disclosure.

### DETAILED DESCRIPTION

### Definitions

Unless defined otherwise, all technical and scientific terms used herein have the same meaning as commonly understood by one of ordinary skill in the art to which this present application belongs. Although any methods and materials similar or equivalent to those described herein can be used in the practice or testing of the present application, illustrative methods and materials are described. As used herein, each of the following terms has the meaning associated with it in this section.

Generally, the nomenclature used herein and the laboratory procedures in molecular biology, pharmacology, and organic chemistry are those well-known and commonly employed in the art.

Standard techniques are used for biochemical and/or biological manipulations. The techniques and procedures are generally performed according to conventional methods in the art and various general references (e.g., Sambrook and Russell, 2012, Molecular Cloning, A Laboratory Approach, Cold Spring Harbor Press, Cold Spring Harbor, NY, and Ausubel et al., 2002, Current Protocols in Molecular Biology, John Wiley & Sons, NY), which are provided throughout this document.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +50%, +40%, +30%, +20%, +10%, +5%, +2% or +1%.

A disease or disorder is “alleviated” if the severity or frequency of at least one sign or symptom of the disease or disorder experienced by a patient is reduced.

As used herein, the terms “analog.” “analogue,” or “derivative” are meant to refer to a chemical compound or molecule made from a parent compound or molecule by one or more chemical reactions. As such, an analog can be a structure having a structure similar to that of the small molecule inhibitors described herein or can be based on a scaffold of a small molecule inhibitor described herein, but differing from it in respect to certain components or structural makeup, which may have a similar or opposite action metabolically.

As used herein, the term “binding” refers to the adherence of molecules to one another, such as, but not limited to, enzymes to substrates, antibodies to antigens, DNA strands to their complementary strands. Binding occurs because the shape and chemical nature of parts of the molecule surfaces are complementary. A common metaphor is the “lock-and-key” used to describe how enzymes fit around their substrate.

An “effective amount” or “therapeutically effective amount” of a compound or composition is that amount of compound or composition that is sufficient to provide a beneficial effect to the subject to which the compound or composition is administered. An “effective amount” of a delivery vehicle is that amount sufficient to effectively bind or deliver a compound or composition.

The phrase “inhibit,” as used herein, means to reduce a molecule, a reaction, an interaction, a gene, an mRNA, and/or a protein's expression, stability, function or activity by a measurable amount or to prevent entirely. Inhibitors are compounds that, e.g., bind to, partially or totally block stimulation, decrease, prevent, delay activation, inactivate, desensitize, or down-regulate a protein, a gene, and an mRNA stability, expression, function and activity, e.g., antagonists.

“Naturally occurring” as applied to an object refers to the fact that the object can be found in nature. For example, a polypeptide or polynucleotide sequence that is present in an organism (including viruses) that can be isolated from a source in nature and which has not been intentionally modified by man is a naturally-occurring sequence.

As used herein, the term “NMDA” refers to N-methyl-D-aspartate.

As used herein, the term “NMDAR” or “NMDA-R” refers to a NMDA glutamate receptor.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or composition useful within the present application within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound or composition useful within the present application, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth: malt: gelatin: talc: excipients, such as cocoa butter and suppository waxes: oils, such as peanut oil, cottonseed oil, safflower oil, sesame oil, olive oil, corn oil and soybean oil: glycols, such as propylene glycol: polyols, such as glycerin, sorbitol, mannitol and polyethylene glycol: esters, such as ethyl oleate and ethyl laurate: agar: buffering agents, such as magnesium hydroxide and aluminum hydroxide: surface active agents: alginic acid: pyrogen-free water: isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound or composition useful within the present application, and are physiologically acceptable to the patient. Supplementary active compounds may also be incorporated into the compositions. The “pharmaceutically acceptable carrier” may further include a pharmaceutically acceptable salt of the compound useful within the present application. Other additional ingredients that may be included in the pharmaceutical compositions used in the practice of the present application are known in the art and described, for example in Remington's Pharmaceutical Sciences (Genaro, Ed., Mack Publishing Co., 1985, Easton, PA), which is incorporated herein by reference.

As used herein, the language “pharmaceutically acceptable salt” or “therapeutically acceptable salt” refers to a salt of the administered compounds prepared from pharmaceutically acceptable non-toxic acids, including inorganic acids or bases, organic acids or bases, solvates, hydrates, or clathrates thereof.

The terms “pharmaceutically effective amount” and “effective amount” or “therapeutically effective amount” refer to a nontoxic but sufficient amount of an agent to provide the desired biological result. That result can be reduction and/or alleviation of the signs, symptoms, or causes of a disease or disorder, or any other desired alteration of a biological system. The amount of a compound of the present application that constitutes a “therapeutically effective amount” varies depending on the compound, the disease state and its severity, the age of the patient to be treated, and the like. An appropriate effective amount in any individual case may be determined by one of ordinary skill in the art using routine experimentation.

As used herein, the terms “polypeptide,” “protein,” and “peptide” are used interchangeably and refer to a polymer composed of amino acid residues, related naturally occurring structural variants, and synthetic non-naturally occurring analogs thereof linked via peptide bonds. Synthetic polypeptides can be synthesized, for example, using an automated polypeptide synthesizer.

By the term “specifically binds,” as used herein, is meant a molecule, such as an antibody, which recognizes and binds to another molecule or feature, but does not substantially recognize or bind other molecules or features in a sample.

A “therapeutic” treatment is a treatment administered to a subject who exhibits signs of pathology, for the purpose of ameliorating, diminishing, and/or eliminating those signs.

As used herein, “treating a disease or disorder” means reducing the frequency with which a symptom of the disease or disorder is experienced by a patient. Disease and disorder are used interchangeably herein. Thus, in a non-limiting embodiment, “treating” or “treatment” of a state, disorder or condition includes: (i) inhibiting the state, disorder or condition, i.e., arresting or reducing the development of the disease or at least one clinical or subclinical symptom thereof, or (ii) relieving the disease, i.e. causing regression of the state, disorder or condition or at least one of its clinical or subclinical symptoms.

As used herein, the term “preventing” encompasses prophylaxis. Accordingly, in a non-limiting embodiment, “preventing” a state, disorder or condition includes preventing or delaying the appearance of clinical symptoms of the state, disorder or condition developing in a subject that may be afflicted with or predisposed to the state, disorder or condition but does not yet experience or display clinical or subclinical symptoms of the state, disorder or condition.

Ranges: throughout this disclosure, various aspects of the present application can be presented in a range format. It should be understood that the description in range format is merely for convenience and brevity and should not be construed as an inflexible limitation on the scope of the present application. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### DESCRIPTION

As demonstrated herein, in one aspect the present application describes a pharmaceutical composition comprising a NMDA receptor modulator and a μ-opioid receptor modulator. The present application provides methods for treating and/or preventing diseases, such as but not limited to depressive disorders, with the combination of at least one NMDA glutamate receptor modulator and at least one μ-opioid receptor modulator. As compared to the NMDA modulator treatment alone, this combination treatment has several benefits, such as but not limited to (1) reducing and/or preventing NMDA antagonist's abuse liability; and/or (2) treating and/or preventing comorbid addictive disorders in depressed patients.

In one aspect, the present application provides a method for treating, ameliorating, and/or preventing depressive disorders, such as but not limited to major depressive disorder, major depressive episode in bipolar disorder (bipolar depression), bipolar I disorder, bipolar II disorder, persistent depressive disorder (dysthymia), disruptive mood dysregulation disorder, major depressive episode, premenstrual dysphoric disorder, substance/medication-induced depressive disorder, depressive disorder due to another medical condition, other specified depressive disorder, unspecified depressive disorder, anxiety disorder, generalized anxiety disorder, social anxiety disorder (social phobia), specific phobia, panic disorder, agoraphobia, separation anxiety disorder, selective mutism, substance-induced anxiety disorder, medication-induced anxiety disorder, anxiety disorder due to another medical condition, borderline personality disorder, treatment-resistant depression, unspecified anxiety disorder, and posttraumatic stress disorder. In some embodiments, the subject suffering from the depressive disorder further suffers from an addictive disorder (i.e., the subject has comorbid depressive disorder and addictive disorder).

Major depressive disorder (MDD) is the second leading cause of disease burden among all medical diseases in developed countries, and is projected to be the number one cause of burden of disease in the 2030's worldwide. However, the efficacy of current pharmacotherapy for depression is only modest. The delay of weeks or months before the onset of antidepressant effects of traditional antidepressants is also problematic, particularly given the elevated risk for suicide in this population.

Major depressive disorder (MDD), also known as depression, is a mental disorder characterized by at least two weeks of continuously low mood, and often accompanied by low self-esteem, loss of interest in previously enjoyable activities, low energy, and unexplained pain. MDD negatively affects a person's social life, sleeping or eating habits, and general health, leading to suicide in about 2-7% of affected adults. Up to 60% of suicide victims had experienced MDD or another mood disorder. MDD patients are often treated with counseling and antidepressants, but unfortunately the efficacy of current pharmacotherapy for depression is only modest.

In some embodiments, the subject in need of such treatment is administered a combination of an NMDA receptor modulator and a μ-opioid receptor modulator. In other embodiments, the NMDA receptor modulator and the μ-opioid receptor modulator are co-administered to the subject. In yet other embodiments, the NMDA receptor modulator and the μ-opioid receptor modulator are coformulated. In yet other embodiments, the methods of the present application allow for reducing, minimizing, and/or eliminating abuse potential of the NMDA receptor modulator by the subject. In yet other embodiments, the NMDA receptor modulator and μ-opioid receptor modulator are the only active agents administered to the subject. In yet other embodiments, the NMDA receptor modulator and μ-opioid receptor modulator are the only active agents administered to the subject to treat, ameliorate, and/or prevent any of the diseases or disorders contemplated herein, such as but not limited to depressive disorders.

### NMDA Receptor Modulators

Glutamate is the major excitatory neurotransmitter in the central nervous system (CNS) and also a key player in numerous brain functions. Abnormalities in glutamatergic neurotransmission are involved in the development of psychiatric disorders including schizophrenia, bipolar disorder, or depression. Glutamate receptors are divided into: ionotropic receptors [N-methyl-D-aspartate (NMDA), α-amino-3-hydroxy5-methyl-4-isoxazolepropionic acid (AMPA), kainate] and metabotropic receptors [mGluR (group 1-3)]. Among the ionotropic receptors, the NMDA receptor is considered a major target for the development of new generation antidepressants. The NMDA receptor is an ionotropic glutamate receptor found in nerve cells. The NMDA receptor is an ion tetramer channel formed by two GluN1 subunits and two GluN2 subunits or GluN2 and GluN3 subunits. The activation of NMDA receptor requires the binding of glutamate to the GluN2 subunits and glycine to its GluN1 subunits, and that activation opens a channel in the receptor than enables triggers cations, particularly calcium ions, to enter the neuron, triggering neuronal excitation and initiating many forms of neuroplasticity. As a result, the NMDA receptor plays important roles in initiating and coordinating neural circuit activity as well as playing roles in neurodevelopment, learning and memory, neurotoxicity, and other functions.

Some endogenous and exogenous agents may modulate the NMDA receptor through binding to its different components, several of these have already been investigated in preclinical and clinical studies for their potential antidepressant activity. In certain embodiments, NMDA receptor modulators can evoke different antidepressant profiles. NMDA receptor modulators have a potential of inducing side effects such as psychotomimetic effects and hallucinations which limits its large-scale clinical use.

NMDA receptors are functionally antagonized by a wide range of drugs. One class of these drugs are the competitive NMDA receptor antagonists that compete with glutamate for access to the NMDA receptor, such as D-CPPene and CGS19755 (Selfotel). There are also negative allosteric modulators of NMDA receptors including neurosteroids, ethanol, and nitrous oxide. There are also drugs that reduce NMDA receptor function through partial agonism or antagonism of the glycine co-agonist site of the NMDA receptor, such as D-cycloserine, ACEA-1021, HA-966, and felbamate.

However, the most commonly studied class of drugs are non-competitive NMDA receptor antagonists include (non-limiting): AP5 (also known as R-2-amino-5-phosphonopentanoate), dextrotphan, CERC-301 (also known as MK-0657 or 4-methylbenzyl (3S,4R)-3-fluoro-4-[(2-pyrimidinylamino)methyl]-1-piperidine carboxylate), conantokins, dextromethorphan, dexanabinol, diethyl ether, dizocilpine (also known as MK-801), ketamine, memantine, nitrous oxide, phencyclidine, xenon, remacemide, methoxetamine, agmatine and 4-chlorokynurenine (also known as AV-101: this is a prodrug of NMDA receptor antagonist 7-chlorokynurenic acid). Further, the following compounds are known to have weak NMDA receptor antagonism: amantadine, dextropropoxyphene, ethanol, guaifenisen, huperzine A, ibogaine, ketobemidone, R/L-methadone, and tramadol.

Antagonists of the NMDA receptor, such as ketamine and nitrous oxide, are used as anesthetics for animals and humans, and they are often used as recreational drugs due to their potential to produce euphoria and stimulation, reduce anxiety, and/or alter sensory processing. Further, NMDA receptor antagonists often have cognitive and behavioral effects, such as dissociation, impaired attention, and disrupted learning and memory at elevated subanesthetic dosages.

The S-isomer of ketamine (Esketamine) was approved by the U.S. FDA for the pharmacotherapy of treatment-resistant depression in March 2019. In 2020, the FDA approved Esketamine for the treatment of major depression symptoms complicated by suicidal ideation or behavior. Although R/S-ketamine is not approved for the treatment of major depression, the off-label prescription of this drug has disseminated into clinical practice as well, perhaps because the original descriptions of antidepressant effects of NMDA receptor antagonists involved R/S-ketamine. Further, there is an earlier use of R/S-ketamine in the context of “ketamine psychedelic therapy” reported in the former Soviet Union (Krupitsky). With the approval of Esketamine, ketamine and other NMDA receptor antagonists, including ketam 4-chlorokynurenine and CERC-301, NMDA receptor antagonists are under development for the treatment of mood disorders, including major depressive disorder and treatment-resistant depression.

Any NMDA receptor modulators known in the art is contemplated within the present application. Non-limiting examples of NMDA receptor modulators useful within the methods of the present application are recited herein, and further include any salt, solvate, enantiomer, tautomer, stereoisomer, and/or geometric isomer thereof, and/or any mixtures thereof. In certain embodiments, the NMDA receptor modulator is ketamine (also known as RS-ketamine. R/S-ketamine. (RS)-2-(2-Chlorophenyl)-2-(methylamino)cyclohexanone. CI-581, CL-369, or CM-52372-2). In certain embodiments, the NMDA receptor modulator is R-ketamine (also known as arketamine, PCN-101. HR-071603, or (R)-2-(2-Chlorophenyl)-2-(methylamino)cyclohexanone). In certain embodiments, the NMDA receptor modulator is S-ketamine (also known as esketamine, esketamine hydrochloride. (S)-Ketamine. S (+)-Ketamine, JNJ-54135419, or(S)-2-(2-Chlorophenyl)-2-(methylamino)cyclohexanone). In certain embodiments, the NMDA receptor modulator is nitrous oxide. In certain embodiments, the NMDA receptor modulator is memantine (also known as 3,5-dimethyladamantan-1-amine. Axura, Ebixa, or Namenda). In certain embodiments, the NMDA receptor modulator is amantadine (also known as adamantan-1-amine, 1-Adamantylamine, Gocovri, Symadine, or Symmetrel). In certain embodiments, the NMDA receptor modulator is racemic dextromethorphan (also known as racemic (4bS,8aR,9S)-3-Methoxy-11-methyl-6,7,8,8a,9,10-hexahydro-5H-9,4b-(epiminoethano) phenanthrene). In certain embodiments, the NMDA receptor modulator is dextromethorphan (also known as DXM, Robitussin, Delsym, DM, DexAlone, or Duract). In certain embodiments, the NMDA receptor modulator is lanicemine (also known as AZD6765, or (1S)-1-phenyl-2-pyridin-2-ylethanamine). In certain embodiments, the NMDA receptor modulator is phencyclidine (also known as 1-(1-phenylcyclohexyl) piperidine. CI-395, phenylcyclohexyl piperidine (PCP), “Angel dust”, Sernyl, or Sernylan). In certain embodiments, the NMDA receptor modulator is dizocilpine (also known as MK-801 or (5R,10S)-(+)-5-methyl-10,11-dihydro-5H-dibenzo[a,d|cyclohepten-5,10-imine). In certain embodiments, the NMDA receptor modulator is CERC-301 (also known as MK-0657, 4-Methylbenzyl (3S,4R)-3-fluoro-4-| (2-pyrimidinylamino)methyl]-1-piperidinecarboxylate, or Rislenemdaz). In certain embodiments, the NMDA receptor modulator is CGP 37849 (also known as (E,2R)-2-amino-4-methyl-5-phosphonopent-3-enoic acid, or CGP-40116). In certain embodiments, the NMDA receptor modulator is 1-aminocylopropanecarboxylic acid. In certain embodiments, the NMDA receptor modulator is traxoprodil (also known as CP-101606, or (1S,2S)-1-(4-hydroxyphenyl)-2-(4-hydroxy-4-phenylpiperidino)-1-propanol). In certain embodiments, the NMDA receptor modulator is Ro 25-6981 (also known as (αR,βS)-α-(4-Hydroxyphenyl)-β-methyl-4-(phenylmethyl)-1-piperidinepropanol). In certain embodiments, the NMDA receptor modulator is eliprodil (also known as 1-(4-chlorophenyl)-2-[4-[(4-fluorophenyl)methyl]piperidin-1-yl]ethanol, or SL-82.0715). In certain embodiments, the NMDA receptor modulator is methoxetamine (also known as MXE, or 3-MeO-2′-oxo-PCE). In certain embodiments, the NMDA receptor modulator is CPPene (also known as Midafotel. SDZ EAA 494). In certain embodiments, the NMDA receptor modulator is AP5 (also known as APV. (2R)-amino-5-phosphonovaleric acid, or (2R)-amino-5-phosphonopentanoate). In certain embodiments, the NMDA receptor modulator is AP-7 (also known as 2-amino-7-phosphoheptanoic acid). In certain embodiments, the NMDA receptor modulator is Selfotel (CGS-19755). In certain embodiments, the NMDA receptor modulator is minocycline (also known as Minocin. Minomycin, or Akamin). In certain embodiments, the NMDA receptor modulator is nitromemantine (also known as YQW-36). In certain embodiments, the NMDA receptor modulator is PD-137889 (also known as N-methylhexahydrofluorenamine). In certain embodiments, the NMDA receptor modulator is rolicyclidine (also known as PCPy). In certain embodiments, the NMDA receptor modulator is tenocyclidine (also known as TCP). In certain embodiments, the NMDA receptor modulator is methoxydine (also known as 4-Methoxyphencyclidine, or 4-MeO-PCP). In certain embodiments, the NMDA receptor modulator is tiletamine. In certain embodiments, the NMDA receptor modulator is neramexane. In certain embodiments, the NMDA receptor modulator is etoxadrol (also known as CL-1848C). In certain embodiments, the NMDA receptor modulator is dexoxadrol (also known as Dioxadrol). In certain embodiments, the NMDA receptor modulator is WMS-2539. In certain embodiments, the NMDA receptor modulator is NEFA. In certain embodiments, the NMDA receptor modulator is remacemide (also known as (+)-2-amino-N-[1,2-di(phenyl) propan-2-yl]acetamide, remacemide hydrochloride. FPL 12924AA. PR 934-423, or Ecovia). In certain embodiments, the NMDA receptor modulator is 3-MeO-PCP (also known as 3-Methoxyphencyclidine). In certain embodiments, the NMDA receptor modulator is 8A-PDHQ (also known as 8a-Phenyldecahydroquinoline). In certain embodiments, the NMDA receptor modulator is atomoxetine (also known as (R)-N-Methyl-3-phenyl-3-(o-tolyloxy) propan-1-amine, or Strattera). In certain embodiments, the NMDA receptor modulator is AZD6765. In certain embodiments, the NMDA receptor modulator is agmatine (also known as (4-aminobutyl) guanidine). In certain embodiments, the NMDA receptor modulator is chloroform (also known as trichloromethane). In certain embodiments, the NMDA receptor modulator is delucemine (also known as NPS-1506). In certain embodiments, the NMDA receptor modulator is dextrallorphan. In certain embodiments, the NMDA receptor modulator is dextrorphan. In certain embodiments, the NMDA receptor modulator is diphenidine (also known as 1,2-DEP, DPD, or DND). In certain embodiments, the NMDA receptor modulator is eticyclidine (also known as PCE, or CI-400). In certain embodiments, the NMDA receptor modulator is gacyclidine (also known as GK-11). In certain embodiments, the NMDA receptor modulator is aptiganel (also known as Cerestat, or CNS-1102). In certain embodiments, the NMDA receptor modulator is HU-211 (also known as Dexanabinol, or ETS2101). In certain embodiments, the NMDA receptor modulator is huperzine A (also known as HupA). In certain embodiments, the NMDA receptor modulator is dipeptide D-Phe-L-Tyr. In certain embodiments, the NMDA receptor modulator is ibogaine. In certain embodiments, the NMDA receptor modulator is rhynchophylline. In certain embodiments, the NMDA receptor modulator is rapastinel (also known as INN, GLYX-13, or BV-102). In certain embodiments, the NMDA receptor modulator is NRX-1074 (also known as Apimostinel. AGN-241660), or Threonyl-prolyl-2R-(2-benzyl)-prolyl-threonine amide). In certain embodiments, the NMDA receptor modulator is 7-Chlorokynurenic acid (also known as 7-CKA). In certain embodiments, the NMDA receptor modulator is 4-Chlorokynurenine (also known as 4-Cl-KYN. AV-101, or 3-(4-Chloroanthraniloyl)-DL-alanine). In certain embodiments, the NMDA receptor modulator is 5,7-Dichlorokynurenic acid (also known as DCKA). In certain embodiments, the NMDA receptor modulator is Kynurenic acid (also known as KYNA or KYN). In certain embodiments, the NMDA receptor modulator is TK-40. In certain embodiments, the NMDA receptor modulator is L-Phenylalanine. In certain embodiments, the NMDA receptor modulator is xenon. In certain embodiments, the NMDA receptor modulator is methadone. In certain embodiments, the NMDA receptor modulator is EU1180-438. In certain embodiments, the NMDA receptor modulator is radiprodil. In certain embodiments, the NMDA receptor modulator is Ifenprodil. In certain embodiments, the NMDA receptor modulator is TCN-201. In certain embodiments, the NMDA receptor modulator is MPX-004. In certain embodiments, the NMDA receptor modulator is MPX-007. In certain embodiments, the NMDA receptor modulator is NAB-14. In certain embodiments, the NMDA receptor modulator is EVT-101. In certain embodiments, the NMDA receptor modulator is QNZ-46. In certain embodiments, the NMDA receptor modulator is DQP-1105. In certain embodiments, the NMDA receptor modulator is pregnanolone sulfate (also known as 3α5βS). In certain embodiments, the NMDA receptor modulator is UBP608. In certain embodiments, the NMDA receptor modulator is UBP618. In certain embodiments, the NMDA receptor modulator is UBP551. In certain embodiments, the NMDA receptor modulator is UBP512. In certain embodiments, the NMDA receptor modulator is HA-966.

In certain embodiments, the NMDA receptor modulator is felbamate. In certain embodiments, the NMDA receptor modulator is PEAQX (also known as NVP-AAM077). In certain embodiments, the NMDA receptor modulator is PD0196860. In certain embodiments, the NMDA receptor modulator is RGH896. In certain embodiments, the NMDA receptor modulator is MK0657. In certain embodiments, the NMDA receptor modulator is L701324. In certain embodiments, the NMDA receptor modulator is LY293558. In certain embodiments, the NMDA receptor modulator is LY300164. In certain embodiments, the NMDA receptor modulator is LY246492. In certain embodiments, the NMDA receptor modulator is LY202157. In certain embodiments, the NMDA receptor modulator is NTX-783.

### μ-Opioid Receptor Modulators

The class of NMDA receptor modulators is plagued with high abuse potential, and drug supplies are often misused as recreational drugs. Ketamine (known as K or Special K) is a widely abused substance, especially in southern Asia (for example, China, Taiwan, Hong Kong, Vietnam, and Cambodia) and the United Kingdom. Some data suggest that ketamine is the third most commonly abused drug, after methamphetamine and MDMA. Ketamine abuse is associated with damage to the mucosa of the urinary bladder and nasal passages, as well as with cognitive, behavioral and emotional abnormalities, including persisting psychosis in some heavy users. Nitrous oxide (known as “whippets”), dextromethorphan (known as “Robo-tripping” or “skittling”) and amantadine also are abused. The most highly abused NMDA receptor modulator is ethanol, which produces significant occupancy of NMDA receptor at ethanol doses associated with heavy social drinking (>4 alcohol drinks). The abuse liability of Esketamine contributed to a “Risk Evaluation and Mitigation Strategy.” required by the U.S. Food and Drug Administration that limits Esketamine administration to a clinic setting where it is administered under the direct supervision of a provider, who monitors the patient for at least two hours. The medication is never to be dispensed directly to a patient for home use.

Beyond the abuse liability of NMDA receptor modulators themselves, there are concerns about prescribing NMDA receptor modulators to depressed patients with comorbid substance use disorders. Major depression doubles the risk for substance use disorders, with the 12-month prevalence of any substance use disorder among patients with major depression of 45.3% and the lifetime prevalence of any substance use disorder in this group of 57.9%. A case report of ketamine addiction and worsening of pathological alcohol use developing in the context of ketamine treatment for depression illustrates a risk of ketamine or Esketamine prescription. Esketamine abuse also developed in a small series of patients who received intranasal Esketamine to self-administer between sessions of intravenous ketamine infusion. Concerns that ketamine or Esketamine treatment would lead to ketamine abuse or worsen other addictions has been echoed by several commentators in the literature, including a consensus report from the American Psychiatric Association. This concern has also led to a proposal to create a UK National Registry to monitor ketamine and Esketamine prescriptions.

Patients with active (moderate-to-severe) substance use disorders other than nicotine were typically excluded from the antidepressant clinical trials of R/S-ketamine and they were excluded from the registration trials of Esketamine. Concerns remain about the impact of ketamine exposure on substance abuse disorders as recreational exposure to ketamine in polysubstance users can be associated with medical risk and poor substance abuse outcomes. A large case series reported a high level of ketamine safety overall, but reported a small number of cases of worsening addiction among people with pre-existing addiction complicating their depression. This has led to recommendations that addiction histories be carefully assessed in all patients receiving ketamine or Esketamine for depression the resulting practice of frequent exclusion of patients with active substance use disorders or even histories of substance use disorders from treatment programs providing access to ketamine or Esketamine.

In summary, while the class of NMDAR antagonist, exemplified by ketamine and Esketamine, are ground-breaking treatments for the rapid treatment of depression, particularly treatment-resistant depression, access to these drugs is constrained by concerns about their abuse liability and the possibility that they may worsen other addictions. Such risks limit the contexts in which these drugs may be administered, reduce the number of providers interested in delivering the treatment, and decrease the number of patients eligible to receive the treatment.

Without wishing to be limited by any theory, NMDA receptor modulators bind to the μ-opioid receptor directly. Such binding may contribute to the abuse liability of these substances, but the clinical efficacy of these drugs does not depend on μ-opioid receptor stimulation. Thus, by blocking μ-opioid receptors, μ-opioid receptor modulators reduce the abuse liability of NMDA receptor modulators without compromising their clinical efficacy. Further, μ-opioid receptor antagonists protect against the relapse of addictions. Members of this class, particularly naltrexone, are approved by the U.S. Food and Drug administration for the treatment of opioid use disorder and alcohol use disorder. Naltrexone also has been reported to reduce the consumption of tobacco products. Lastly, as supported by a case report, naltrexone decreases the consumption of ketamine and supports the treatment of ketamine use disorder.

The opium poppy was known to possess powerful analgesic properties even in ancient times. It was not until the 19^{th} century that one of its potent analgesic ingredients, morphine, was successfully isolated. However, morphine was also shown to have adverse effects on both the respiratory and gastrointestinal (GI) systems. Addiction and tolerance caused by this substance led to strict government regulations for its production, use, and distribution. Pharmacological studies later revealed that opioid receptors trigger a series of intracellular responses which are responsible for their pharmacological outcomes. The μ-opioid receptor is a well-known member of this receptor family. Many morphine analogs are believed to target μ-opioid receptor via two distinct downstream signaling pathways that are simultaneously stimulated. These two pathways are independently associated with the analgesic properties and undesired side effects of opioids. Analgesia is achieved via a classical G-protein pathway which suppresses neuronal excitability and promotes the hyperpolarization of neurons. By contrast, most undesirable opioid-mediated effects are related to the β-arrestin pathway, which regulates the desensitization and internalization of the opioid receptor.

Any μ-opioid receptor modulator known in the art is contemplated within the present application. In some embodiments, the μ-opioid receptor modulator is a μ-opioid receptor antagonist. In some embodiments, the μ-opioid receptor modulator is a μ-opioid receptor negative allosteric modulator. In some embodiments, the μ-opioid receptor modulator is a μ-opioid receptor partial agonist.

Non-limiting examples of μ-opioid receptor modulators useful within the methods of the present application are recited herein, and include any salt, solvate, enantiomer, tautomer, stereoisomer, and/or geometric isomer thereof, or any mixtures thereof. In certain embodiments, the μ-opioid receptor modulator is naltrexone (also known as 17-(cyclopropylmethyl)-4,5α-epoxy-3,14-dihydroxymorphinan-6-one). In certain embodiments, the μ-opioid receptor modulator is long-acting naltrexone (extended-release naltrexone, e.g., VIVITROL®). In certain embodiments, the μ-opioid receptor modulator is naloxone (also known as (4R,4aS,7aR,12bS)-4α,9-dihydroxy-3-prop-2-enyl-2,4,5,6,7a, 13-hexahydro-1H-4,12-methanobenzofuro [3,2-e] isoquinoline-7-one, Narcan, Evzio, or Nyxoid). In certain embodiments, the μ-opioid receptor modulator is long-acting naloxone (extended-release naloxone). In certain embodiments, the μ-opioid receptor modulator is nalmefene (also known as 17-cyclopropylmethyl-4,5a-epoxy-6-methylenemorphinan-3,14-diol, nalmetrene, or Selincro). In certain embodiments, the μ-opioid receptor modulator is long-acting nalmefene (extended-release nalmefene). In certain embodiments, the μ-opioid receptor modulator is nalodeine (also known as N-allylnorcodeine, or (4R,4aR,7S,7aR,12bS)-9-methoxy-3-prop-2-enyl-2,4,4α, 7,7α, 13-hexahydro-1H-4,12-methanobenzofuro[3,2-e]isoquinoline-7-ol). In some embodiments, the long-acting naltrexone has a longer serum half-life than naltrexone. In some embodiments, the long-acting naloxone has a longer serum half-life than naloxone. In some embodiments, the long-acting nalmefene has a longer serum half-life than nalmefene.

### Salts and Isoforms

The compounds described herein may form salts with acids and/or bases, and such salts are included in the present application. In some embodiments, the salts are pharmaceutically acceptable salts. The term “salts” embraces addition salts of free acids and/or bases that are useful within the methods of the present application. The term “pharmaceutically acceptable salt” refers to salts that possess toxicity profiles within a range that affords utility in pharmaceutical applications. Pharmaceutically unacceptable salts may nonetheless possess properties such as high crystallinity, which have utility in the practice of the present application, such as for example utility in process of synthesis, purification or formulation of compounds useful within the methods of the present application.

Suitable pharmaceutically acceptable acid addition salts may be prepared from an inorganic acid or from an organic acid. Examples of inorganic acids include hydrochloric, hydrobromic, hydriodic, nitric, carbonic, sulfuric (including sulfate and hydrogen sulfate), and phosphoric acids (including hydrogen phosphate and dihydrogen phosphate). Appropriate organic acids may be selected from aliphatic, cycloaliphatic, aromatic, araliphatic, heterocyclic, carboxylic and sulfonic classes of organic acids, examples of which include formic, acetic, propionic, succinic, glycolic, gluconic, lactic, malic, tartaric, citric, ascorbic, glucuronic, maleic, malonic, saccharin, fumaric, pyruvic, aspartic, glutamic, benzoic, anthranilic, 4-hydroxy benzoic, phenylacetic, mandelic, embonic (pamoic), methanesulfonic, ethanesulfonic, benzenesulfonic, pantothenic, trifluoromethanesulfonic, 2-hydroxyethanesulfonic, p-toluenesulfonic, sulfanilic, cyclohexylaminosulfonic, stearic, alginic, β-hydroxy butyric, salicylic, galactaric and galacturonic acid.

Suitable pharmaceutically acceptable base addition salts of compounds of the present application include, for example, ammonium salts, metallic salts including alkali metal, alkaline earth metal and transition metal salts such as, for example, calcium, magnesium, potassium, sodium and zinc salts. Pharmaceutically acceptable base addition salts also include organic salts made from basic amines such as, for example, N,N′-dibenzylethylene-diamine, chloroprocaine, choline, diethanolamine, ethylenediamine, meglumine (also known as N-methylglucamine) and procaine. All of these salts may be prepared from the corresponding compound by reacting, for example, the appropriate acid or base with the compound.

The compounds of the present application may possess one or more stereocenters, and each stereocenter may exist independently in either the (R) or(S) configuration. In some embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

In some embodiments, the compounds of the present application exist as tautomers. All tautomers are included within the scope of the compounds recited herein.

Compounds described herein also include isotopically labeled compounds wherein one or more atoms is replaced by an atom having the same atomic number, but an atomic mass or mass number different from the atomic mass or mass number usually found in nature. Examples of isotopes suitable for inclusion in the compounds described herein include and are not limited to ²H, ³H, ¹¹C, ¹³C, ¹⁴C, ³⁶Cl, ¹⁸F, ¹²³I, ¹²⁵I, ¹³N, ¹⁵N, ¹⁵O, ¹⁷O, ¹⁸O, ³²P, and ³⁵S. In some embodiments, substitution with heavier isotopes such as deuterium affords greater chemical stability. Isotopically labeled compounds are prepared by any suitable method or by processes using an appropriate isotopically labeled reagent in place of the non-labeled reagent otherwise employed.

In some embodiments, the compounds described herein are labeled by other means, including, but not limited to, the use of chromophores or fluorescent moieties, bioluminescent labels, or chemiluminescent labels.

### Pharmaceutical Compositions

Described herein is a pharmaceutical composition comprising a N-methyl-D-aspartate receptor modulator and a μ-opioid receptor modulator. In some embodiments, the NMDA receptor modulator is selected from the group consisting of a NMDA receptor antagonist, a NMDA receptor negative allosteric modulator, and a NMDA receptor partial agonist. In some embodiments, the μ-opioid receptor modulator is selected from the group consisting of a μ-opioid receptor antagonist, a μ-opioid receptor negative allosteric modulator, and a μ-opioid receptor partial agonist.

In some embodiments, the % occupancy of the μ-opioid receptors in vivo and/or in vitro is measured by positron emission tomography with a [¹¹C]-carfentanil ligand.

In some embodiments, the μ-opioid receptor modulator is present in an amount that occupies at least about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, and/or 99.9% of μ-opioid receptors in vivo as measured by positron emission tomography with a [¹¹C]-carfentanil ligand. In some embodiments, the μ-opioid receptor modulator is present in an amount that occupies at least 10% of μ-opioid receptors in vivo as measured by positron emission tomography with a [¹¹C]-carfentanil ligand. In some embodiments, the μ-opioid receptor modulator is present in an amount that occupies at least 25% of the μ-opioid receptors in vivo. In some embodiments, the μ-opioid receptor modulator is present in an amount that occupies at least 50% of the μ-opioid receptors in vivo. In some embodiments, the μ-opioid receptor modulator is present in an amount that occupies at least 75% of the μ-opioid receptors in vivo. In some embodiments, the μ-opioid receptor modulator is present in an amount that occupies at least 90% of the μ-opioid receptors in vivo. In some embodiments, the μ-opioid receptor modulator is present in an amount that occupies at least 99% of the μ-opioid receptors in vivo.

The pharmaceutical compositions described herein can comprise the μ-opioid receptor modulator in various amounts. In some embodiments, the μ-opioid receptor modulator is present in an amount of less than about 400 mg. In some embodiments, the μ-opioid receptor modulator is present in an amount of less than about 350 mg. In some embodiments, the μ-opioid receptor modulator is present in an amount of less than about 300 mg. In some embodiments, the μ-opioid receptor modulator is present in an amount of less than about 250 mg. In some embodiments, the μ-opioid receptor modulator is present in an amount of less than about 200 mg. In some embodiments, the μ-opioid receptor modulator is present in an amount of less than about 150 mg. In some embodiments, the μ-opioid receptor modulator is present in an amount of less than about 100 mg. In some embodiments, the μ-opioid receptor modulator is present in an amount of less than about 90 mg. In some embodiments, the μ-opioid receptor modulator is present in an amount of less than about 80 mg. In some embodiments, the μ-opioid receptor modulator is present in an amount of less than about 70 mg. In some embodiments, the μ-opioid receptor modulator is present in an amount of less than about 60 mg. In some embodiments, the μ-opioid receptor modulator is present in an amount of less than about 50 mg. In some embodiments, the μ-opioid receptor modulator is present in an amount of less than about 40 mg. In some embodiments, the μ-opioid receptor modulator is present in an amount of less than about 30 mg. In some embodiments, the μ-opioid receptor modulator is present in an amount of less than about 20 mg. In some embodiments, the μ-opioid receptor modulator is present in an amount of less than about 10 mg. In some embodiments, the μ-opioid receptor modulator is present in an amount of less than about 5 mg. In some embodiments, the μ-opioid receptor modulator is present in an amount of greater than about 400 mg. In some embodiments, the μ-opioid receptor modulator is present in an amount of greater than about 350 mg. In some embodiments, the μ-opioid receptor modulator is present in an amount of greater than about 300 mg. In some embodiments, the μ-opioid receptor modulator is present in an amount of greater than about 250 mg. In some embodiments, the μ-opioid receptor modulator is present in an amount of greater than about 200 mg. In some embodiments, the μ-opioid receptor modulator is present in an amount of greater than about 150 mg. In some embodiments, the μ-opioid receptor modulator is present in an amount of greater than about 100 mg. In some embodiments, the μ-opioid receptor modulator is present in an amount of greater than about 90 mg. In some embodiments, the μ-opioid receptor modulator is present in an amount of greater than about 80 mg. In some embodiments, the μ-opioid receptor modulator is present in an amount of greater than about 70 mg. In some embodiments, the μ-opioid receptor modulator is present in an amount of greater than about 60 mg. In some embodiments, the μ-opioid receptor modulator is present in an amount of greater than about 50 mg. In some embodiments, the μ-opioid receptor modulator is present in an amount of greater than about 40 mg. In some embodiments, the μ-opioid receptor modulator is present in an amount of greater than about 30 mg. In some embodiments, the μ-opioid receptor modulator is present in an amount of greater than about 20 mg. In some embodiments, the μ-opioid receptor modulator is present in an amount of greater than about 10 mg. In some embodiments, the μ-opioid receptor modulator is present in an amount of greater than about 5 mg.

In some embodiments, the μ-opioid receptor modulator is present in an amount of about 1 mg to about 5 mg. In some embodiments, the μ-opioid receptor modulator is present in an amount of about 1 mg to about 10 mg. In some embodiments, the μ-opioid receptor modulator is present in an amount of about 1 mg to about 20 mg. In some embodiments, the μ-opioid receptor modulator is present in an amount of about 1 mg to about 50 mg. In some embodiments, the μ-opioid receptor modulator is present in an amount of about 1 mg to about 80 mg. In some embodiments, the μ-opioid receptor modulator is present in an amount of about 1 mg to about 100 mg. In some embodiments, the μ-opioid receptor modulator is present in an amount of about 1 mg to about 150 mg. In some embodiments, the μ-opioid receptor modulator is present in an amount of about 1 mg to about 200 mg. In some embodiments, the μ-opioid receptor modulator is present in an amount of about 1 mg to about 250 mg. In some embodiments, the μ-opioid receptor modulator is present in an amount of about 1 mg to about 300 mg. In some embodiments, the μ-opioid receptor modulator is present in an amount of about 1 mg to about 350 mg. In some embodiments, the μ-opioid receptor modulator is present in an amount of about 1 mg to about 400 mg. In some embodiments, the μ-opioid receptor modulator is present in an amount of about 5 mg to about 10 mg. In some embodiments, the μ-opioid receptor modulator is present in an amount of about 5 mg to about 20 mg. In some embodiments, the μ-opioid receptor modulator is present in an amount of about 5 mg to about 50 mg. In some embodiments, the μ-opioid receptor modulator is present in an amount of about 5 mg to about 80 mg. In some embodiments, the μ-opioid receptor modulator is present in an amount of about 5 mg to about 100 mg. In some embodiments, the μ-opioid receptor modulator is present in an amount of about 5 mg to about 150 mg. In some embodiments, the μ-opioid receptor modulator is present in an amount of about 5 mg to about 200 mg. In some embodiments, the μ-opioid receptor modulator is present in an amount of about 5 mg to about 250 mg. In some embodiments, the μ-opioid receptor modulator is present in an amount of about 5 mg to about 300 mg. In some embodiments, the μ-opioid receptor modulator is present in an amount of about 5 mg to about 350 mg. In some embodiments, the μ-opioid receptor modulator is present in an amount of about 5 mg to about 400 mg. In some embodiments, the μ-opioid receptor modulator is present in an amount of about 10 mg to about 20 mg. In some embodiments, the μ-opioid receptor modulator is present in an amount of about 10 mg to about 50 mg. In some embodiments, the μ-opioid receptor modulator is present in an amount of about 10 mg to about 80 mg. In some embodiments, the μ-opioid receptor modulator is present in an amount of about 10 mg to about 100 mg. In some embodiments, the μ-opioid receptor modulator is present in an amount of about 10 mg to about 150 mg. In some embodiments, the μ-opioid receptor modulator is present in an amount of about 10 mg to about 200 mg. In some embodiments, the μ-opioid receptor modulator is present in an amount of about 10 mg to about 250 mg. In some embodiments, the μ-opioid receptor modulator is present in an amount of about 10 mg to about 300 mg. In some embodiments, the μ-opioid receptor modulator is present in an amount of about 10 mg to about 350 mg. In some embodiments, the μ-opioid receptor modulator is present in an amount of about 10 mg to about 400 mg. In some embodiments, the μ-opioid receptor modulator is present in an amount of about 20 mg to about 50 mg. In some embodiments, the μ-opioid receptor modulator is present in an amount of about 20 mg to about 80 mg. In some embodiments, the μ-opioid receptor modulator is present in an amount of about 20 mg to about 100 mg. In some embodiments, the μ-opioid receptor modulator is present in an amount of about 20 mg to about 150 mg. In some embodiments, the μ-opioid receptor modulator is present in an amount of about 20 mg to about 200 mg. In some embodiments, the μ-opioid receptor modulator is present in an amount of about 20 mg to about 250 mg. In some embodiments, the μ-opioid receptor modulator is present in an amount of about 20 mg to about 300 mg. In some embodiments, the μ-opioid receptor modulator is present in an amount of about 20 mg to about 350 mg. In some embodiments, the μ-opioid receptor modulator is present in an amount of about 20 mg to about 400 mg. In some embodiments, the μ-opioid receptor modulator is present in an amount of about 50 mg to about 80 mg. In some embodiments, the μ-opioid receptor modulator is present in an amount of about 50 mg to about 100 mg. In some embodiments, the μ-opioid receptor modulator is present in an amount of about 50 mg to about 150 mg. In some embodiments, the μ-opioid receptor modulator is present in an amount of about 50 mg to about 200 mg. In some embodiments, the μ-opioid receptor modulator is present in an amount of about 50 mg to about 250 mg. In some embodiments, the μ-opioid receptor modulator is present in an amount of about 50 mg to about 300 mg. In some embodiments, the μ-opioid receptor modulator is present in an amount of about 50 mg to about 350 mg. In some embodiments, the μ-opioid receptor modulator is present in an amount of about 50 mg to about 400 mg. In some embodiments, the μ-opioid receptor modulator is present in an amount of about 80 mg to about 100 mg. In some embodiments, the μ-opioid receptor modulator is present in an amount of about 80 mg to about 150 mg. In some embodiments, the μ-opioid receptor modulator is present in an amount of about 80 mg to about 200 mg. In some embodiments, the μ-opioid receptor modulator is present in an amount of about 80 mg to about 250 mg. In some embodiments, the μ-opioid receptor modulator is present in an amount of about 80 mg to about 300 mg. In some embodiments, the μ-opioid receptor modulator is present in an amount of about 80 mg to about 350 mg. In some embodiments, the μ-opioid receptor modulator is present in an amount of about 80 mg to about 400 mg. In some embodiments, the μ-opioid receptor modulator is present in an amount of about 100 mg to about 150 mg. In some embodiments, the μ-opioid receptor modulator is present in an amount of about 100 mg to about 200 mg. In some embodiments, the μ-opioid receptor modulator is present in an amount of about 100 mg to about 250 mg. In some embodiments, the μ-opioid receptor modulator is present in an amount of about 100 mg to about 300 mg. In some embodiments, the μ-opioid receptor modulator is present in an amount of about 100 mg to about 350 mg. In some embodiments, the μ-opioid receptor modulator is present in an amount of about 100 mg to about 400 mg. In some embodiments, the μ-opioid receptor modulator is present in an amount of about 150 mg to about 200 mg. In some embodiments, the μ-opioid receptor modulator is present in an amount of about 150 mg to about 250 mg. In some embodiments, the μ-opioid receptor modulator is present in an amount of about 150 mg to about 300 mg. In some embodiments, the μ-opioid receptor modulator is present in an amount of about 150 mg to about 350 mg. In some embodiments, the μ-opioid receptor modulator is present in an amount of about 150 mg to about 400 mg. In some embodiments, the μ-opioid receptor modulator is present in an amount of about 200 mg to about 250 mg. In some embodiments, the μ-opioid receptor modulator is present in an amount of about 200 mg to about 300 mg. In some embodiments, the μ-opioid receptor modulator is present in an amount of about 200 mg to about 350 mg. In some embodiments, the μ-opioid receptor modulator is present in an amount of about 200 mg to about 400 mg. In some embodiments, the μ-opioid receptor modulator is present in an amount of about 250 mg to about 300 mg. In some embodiments, the μ-opioid receptor modulator is present in an amount of about 250 mg to about 350 mg. In some embodiments, the μ-opioid receptor modulator is present in an amount of about 250 mg to about 400 mg. In some embodiments, the μ-opioid receptor modulator is present in an amount of about 300 mg to about 350 mg. In some embodiments, the μ-opioid receptor modulator is present in an amount of about 350 mg to about 400 mg.

The pharmaceutical compositions described herein can comprise the NMDA receptor modulator in various amounts. In some embodiments, the NMDA receptor modulator is present in an amount of less than about 150 mg. In some embodiments, the NMDA receptor modulator is present in an amount of less than about 100 mg. In some embodiments, the NMDA receptor modulator is present in an amount of less than about 90 mg. In some embodiments, the NMDA receptor modulator is present in an amount of less than about 80 mg. In some embodiments, the NMDA receptor modulator is present in an amount of less than about 70 mg. In some embodiments, the NMDA receptor modulator is present in an amount of less than about 60 mg. In some embodiments, the NMDA receptor modulator is present in an amount of less than about 50 mg. In some embodiments, the NMDA receptor modulator is present in an amount of less than about 40 mg. In some embodiments, the NMDA receptor modulator is present in an amount of less than about 30 mg, less than about 20 mg. In some embodiments, the NMDA receptor modulator is present in an amount of less than about 10 mg. In some embodiments, the NMDA receptor modulator is present in an amount of less than about 5 mg. In some embodiments, the NMDA receptor modulator is present in an amount of greater than about 150 mg. In some embodiments, the NMDA receptor modulator is present in an amount of greater than about 100 mg. In some embodiments, the NMDA receptor modulator is present in an amount of greater than about 90 mg. In some embodiments, the NMDA receptor modulator is present in an amount of greater than about 80 mg. In some embodiments, the NMDA receptor modulator is present in an amount of greater than about 70 mg. In some embodiments, the NMDA receptor modulator is present in an amount of greater than about 60 mg. In some embodiments, the NMDA receptor modulator is present in an amount of greater than about 50 mg. In some embodiments, the NMDA receptor modulator is present in an amount of greater than about 40 mg. In some embodiments, the NMDA receptor modulator is present in an amount of greater than about 30 mg, greater than about 20 mg. In some embodiments, the NMDA receptor modulator is present in an amount of greater than about 10 mg. In some embodiments, the NMDA receptor modulator is present in an amount of greater than about 5 mg.

In some embodiments, the NMDA receptor modulator is present in an amount of about 1 mg to about 5 mg. In some embodiments, the NMDA receptor modulator is present in an amount of about 1 mg to about 10 mg. In some embodiments, the NMDA receptor modulator is present in an amount of about 1 mg to about 20 mg. In some embodiments, the NMDA receptor modulator is present in an amount of about 1 mg to about 50 mg. In some embodiments, the NMDA receptor modulator is present in an amount of about 1 mg to about 40 mg. In some embodiments, the NMDA receptor modulator is present in an amount of about 1 mg to about 50 mg. In some embodiments, the NMDA receptor modulator is present in an amount of about 1 mg to about 60 mg. In some embodiments, the NMDA receptor modulator is present in an amount of about 1 mg to about 70 mg. In some embodiments, the NMDA receptor modulator is present in an amount of about 1 mg to about 80 mg. In some embodiments, the NMDA receptor modulator is present in an amount of about 1 mg to about 90 mg. In some embodiments, the NMDA receptor modulator is present in an amount of about 1 mg to about 100 mg. In some embodiments, the NMDA receptor modulator is present in an amount of about 1 mg to about 150 mg. In some embodiments, the NMDA receptor modulator is present in an amount of about 5 mg to about 10 mg. In some embodiments, the NMDA receptor modulator is present in an amount of about 5 mg to about 20 mg. In some embodiments, the NMDA receptor modulator is present in an amount of about 5 mg to about 50 mg. In some embodiments, the NMDA receptor modulator is present in an amount of about 5 mg to about 40 mg. In some embodiments, the NMDA receptor modulator is present in an amount of about 5 mg to about 50 mg. In some embodiments, the NMDA receptor modulator is present in an amount of about 5 mg to about 60 mg. In some embodiments, the NMDA receptor modulator is present in an amount of about 5 mg to about 70 mg. In some embodiments, the NMDA receptor modulator is present in an amount of about 5 mg to about 80 mg. In some embodiments, the NMDA receptor modulator is present in an amount of about 5 mg to about 90 mg. In some embodiments, the NMDA receptor modulator is present in an amount of about 5 mg to about 100 mg. In some embodiments, the NMDA receptor modulator is present in an amount of about 5 mg to about 150 mg. In some embodiments, the NMDA receptor modulator is present in an amount of about 10 mg to about 20 mg. In some embodiments, the NMDA receptor modulator is present in an amount of about 10 mg to about 50 mg. In some embodiments, the NMDA receptor modulator is present in an amount of about 10 mg to about 40 mg. In some embodiments, the NMDA receptor modulator is present in an amount of about 10 mg to about 50 mg. In some embodiments, the NMDA receptor modulator is present in an amount of about 10 mg to about 60 mg. In some embodiments, the NMDA receptor modulator is present in an amount of about 10 mg to about 70 mg. In some embodiments, the NMDA receptor modulator is present in an amount of about 10 mg to about 80 mg. In some embodiments, the NMDA receptor modulator is present in an amount of about 10 mg to about 90 mg. In some embodiments, the NMDA receptor modulator is present in an amount of about 10 mg to about 100 mg. In some embodiments, the NMDA receptor modulator is present in an amount of about 10 mg to about 150 mg. In some embodiments, the NMDA receptor modulator is present in an amount of about 20 mg to about 50 mg. In some embodiments, the NMDA receptor modulator is present in an amount of about 20 mg to about 40 mg. In some embodiments, the NMDA receptor modulator is present in an amount of about 20 mg to about 50 mg. In some embodiments, the NMDA receptor modulator is present in an amount of about 20 mg to about 60 mg. In some embodiments, the NMDA receptor modulator is present in an amount of about 20 mg to about 70 mg. In some embodiments, the NMDA receptor modulator is present in an amount of about 20 mg to about 80 mg. In some embodiments, the NMDA receptor modulator is present in an amount of about 20 mg to about 90 mg. In some embodiments, the NMDA receptor modulator is present in an amount of about 20 mg to about 100 mg. In some embodiments, the NMDA receptor modulator is present in an amount of about 20 mg to about 150 mg. In some embodiments, the NMDA receptor modulator is present in an amount of about 30 mg to about 40 mg. In some embodiments, the NMDA receptor modulator is present in an amount of about 30 mg to about 50 mg. In some embodiments, the NMDA receptor modulator is present in an amount of about 30 mg to about 60 mg. In some embodiments, the NMDA receptor modulator is present in an amount of about 30 mg to about 70 mg. In some embodiments, the NMDA receptor modulator is present in an amount of about 30 mg to about 80 mg. In some embodiments, the NMDA receptor modulator is present in an amount of about 30 mg to about 90 mg. In some embodiments, the NMDA receptor modulator is present in an amount of about 30 mg to about 100 mg. In some embodiments, the NMDA receptor modulator is present in an amount of about 30 mg to about 150 mg. In some embodiments, the NMDA receptor modulator is present in an amount of about 40 mg to about 50 mg. In some embodiments, the NMDA receptor modulator is present in an amount of about 40 mg to about 60 mg. In some embodiments, the NMDA receptor modulator is present in an amount of about 40 mg to about 70 mg. In some embodiments, the NMDA receptor modulator is present in an amount of about 40 mg to about 80 mg. In some embodiments, the NMDA receptor modulator is present in an amount of about 40 mg to about 90 mg. In some embodiments, the NMDA receptor modulator is present in an amount of about 40 mg to about 100 mg. In some embodiments, the NMDA receptor modulator is present in an amount of about 40 mg to about 150 mg. In some embodiments, the NMDA receptor modulator is present in an amount of about 50 mg to about 60 mg. In some embodiments, the NMDA receptor modulator is present in an amount of about 50 mg to about 70 mg. In some embodiments, the NMDA receptor modulator is present in an amount of about 50 mg to about 80 mg. In some embodiments, the NMDA receptor modulator is present in an amount of about 50 mg to about 90 mg. In some embodiments, the NMDA receptor modulator is present in an amount of about 50 mg to about 100 mg. In some embodiments, the NMDA receptor modulator is present in an amount of about 50 mg to about 150 mg. In some embodiments, the NMDA receptor modulator is present in an amount of about 60 mg to about 70 mg. In some embodiments, the NMDA receptor modulator is present in an amount of about 60 mg to about 80 mg. In some embodiments, the NMDA receptor modulator is present in an amount of about 60 mg to about 90 mg. In some embodiments, the NMDA receptor modulator is present in an amount of about 60 mg to about 100 mg. In some embodiments, the NMDA receptor modulator is present in an amount of about 60 mg to about 150 mg. In some embodiments, the NMDA receptor modulator is present in an amount of about 70 mg to about 80 mg. In some embodiments, the NMDA receptor modulator is present in an amount of about 70 mg to about 90 mg. In some embodiments, the NMDA receptor modulator is present in an amount of about 70 mg to about 100 mg. In some embodiments, the NMDA receptor modulator is present in an amount of about 70 mg to about 150 mg. In some embodiments, the NMDA receptor modulator is present in an amount of about 80 mg to about 90 mg. In some embodiments, the NMDA receptor modulator is present in an amount of about 80 mg to about 100 mg. In some embodiments, the NMDA receptor modulator is present in an amount of about 80 mg to about 150 mg. In some embodiments, the NMDA receptor modulator is present in an amount of about 90 mg to about 100 mg. In some embodiments, the NMDA receptor modulator is present in an amount of about 90 mg to about 150 mg. In some embodiments, the NMDA receptor modulator is present in an amount of about 100 mg to about 150 mg.

The pharmaceutical compositions described herein comprises at least one pharmaceutically acceptable carrier and one or more pharmaceutically active agents contemplated herein. The pharmaceutical compositions described herein may be prepared by any method known or hereafter developed in the art of pharmacology. In general, such preparatory methods include bringing the active ingredient into association with a carrier or one or more other accessory ingredients, and then, if necessary or desirable, shaping or packaging the product into a desired single- or multi-dose unit.

In some embodiments, the NMDA receptor modulator and the μ-opioid receptor modulator are coformulated in a pharmaceutical composition. In other embodiments, the NMDA receptor modulator and the μ-opioid receptor modulator are coformulated in a way whereby physical separation of the NMDA receptor modulator and the μ-opioid receptor modulator is not possible and/or feasible. In yet other embodiments, separation of the NMDA receptor modulator and the μ-opioid receptor modulator requires chemical purification (using extractive procedures, chromatographic separation and the like). In yet other embodiments, separation of the NMDA receptor modulator and the μ-opioid receptor modulator requires denaturation and/or destruction of the pharmaceutical composition.

In some embodiments, the pharmaceutical compositions of the present application comprise a NMDA receptor modulator and a μ-opioid receptor modulator, wherein the NMDA receptor modulator and the μ-opioid receptor modulator are present in amounts whereby: administration of the pharmaceutical compositions to a subject treats in the subject a disease or disorder that is treatable or preventable by the NMDA receptor modulator; and the μ-opioid receptor modulator prevents and/or minimizes abuse of the NMDA receptor modulator by the subject.

### Methods of Use

The present application provides a method of treating, ameliorating/and/or preventing a disease or disorder in a subject in need thereof, comprising administering a NMDA receptor modulator and a μ-opioid receptor modulator to the subject. The present application further provides a method of preventing and/or minimizing abuse of a NMDA receptor modulator by a subject. The present application further provides a method of treating, ameliorating, and/or preventing major depressive disorder in a subject. In some embodiments, the NMDA receptor modulator is selected from the group consisting of a NMDA receptor antagonist, a NMDA receptor negative allosteric modulator, and a NMDA receptor partial agonist. In some embodiments, the μ-opioid receptor modulator is selected from the group consisting of a μ-opioid receptor antagonist, a μ-opioid receptor negative allosteric modulator, and a μ-opioid receptor partial agonist.

In some embodiments, the method comprises administering to the subject a therapeutically effective amount of the NMDA receptor modulator and a therapeutically effective amount of a μ-opioid receptor modulator.

In some embodiments, the % occupancy of the μ-opioid receptors in vivo and/or in vitro is measured by positron emission tomography with a [¹¹C]-carfentanil ligand. In some embodiments, the μ-opioid receptor modulator is present in an amount that occupies at least about 10% of μ-opioid receptors in vivo as measured by positron emission tomography with a [¹¹C]-carfentanil ligand. In some embodiments, the μ-opioid receptor modulator is present in an amount that occupies at least about 15% of μ-opioid receptors in vivo as measured by positron emission tomography with a [¹¹C]-carfentanil ligand. In some embodiments, the μ-opioid receptor modulator is present in an amount that occupies at least about 20% of μ-opioid receptors in vivo as measured by positron emission tomography with a [¹¹C]-carfentanil ligand. In some embodiments, the μ-opioid receptor modulator is present in an amount that occupies at least about 25% of μ-opioid receptors in vivo as measured by positron emission tomography with a [¹¹C]-carfentanil ligand. In some embodiments, the μ-opioid receptor modulator is present in an amount that occupies at least about 30% of μ-opioid receptors in vivo as measured by positron emission tomography with a [¹¹C]-carfentanil ligand. In some embodiments, the μ-opioid receptor modulator is present in an amount that occupies at least about 35% of μ-opioid receptors in vivo as measured by positron emission tomography with a [¹¹C]-carfentanil ligand. In some embodiments, the μ-opioid receptor modulator is present in an amount that occupies at least about 40% of μ-opioid receptors in vivo as measured by positron emission tomography with a [¹¹C]-carfentanil ligand. In some embodiments, the μ-opioid receptor modulator is present in an amount that occupies at least about 45% of μ-opioid receptors in vivo as measured by positron emission tomography with a [¹¹C]-carfentanil ligand. In some embodiments, the μ-opioid receptor modulator is present in an amount that occupies at least about 50% of μ-opioid receptors in vivo as measured by positron emission tomography with a [¹¹C]-carfentanil ligand. In some embodiments, the μ-opioid receptor modulator is present in an amount that occupies at least about 55% of μ-opioid receptors in vivo as measured by positron emission tomography with a [¹¹C]-carfentanil ligand. In some embodiments, the μ-opioid receptor modulator is present in an amount that occupies at least about 60% of μ-opioid receptors in vivo as measured by positron emission tomography with a [¹¹C]-carfentanil ligand. In some embodiments, the μ-opioid receptor modulator is present in an amount that occupies at least about 65% of μ-opioid receptors in vivo as measured by positron emission tomography with a [¹¹C]-carfentanil ligand. In some embodiments, the μ-opioid receptor modulator is present in an amount that occupies at least about 70% of μ-opioid receptors in vivo as measured by positron emission tomography with a [¹¹C]-carfentanil ligand. In some embodiments, the μ-opioid receptor modulator is present in an amount that occupies at least about 75% of μ-opioid receptors in vivo as measured by positron emission tomography with a [¹¹C]-carfentanil ligand. In some embodiments, the μ-opioid receptor modulator is present in an amount that occupies at least about 80% of μ-opioid receptors in vivo as measured by positron emission tomography with a [¹¹C]-carfentanil ligand. In some embodiments, the μ-opioid receptor modulator is present in an amount that occupies at least about 85% of μ-opioid receptors in vivo as measured by positron emission tomography with a [¹¹C]-carfentanil ligand. In some embodiments, the μ-opioid receptor modulator is present in an amount that occupies at least about 90% of μ-opioid receptors in vivo as measured by positron emission tomography with a [¹¹C]-carfentanil ligand. In some embodiments, the μ-opioid receptor modulator is present in an amount that occupies at least about 91% of μ-opioid receptors in vivo as measured by positron emission tomography with a [¹¹C]-carfentanil ligand. In some embodiments, the μ-opioid receptor modulator is present in an amount that occupies at least about 92% of μ-opioid receptors in vivo as measured by positron emission tomography with a [¹¹C]-carfentanil ligand. In some embodiments, the μ-opioid receptor modulator is present in an amount that occupies at least about 93% of μ-opioid receptors in vivo as measured by positron emission tomography with a [¹¹C]-carfentanil ligand. In some embodiments, the μ-opioid receptor modulator is present in an amount that occupies at least about 94% of μ-opioid receptors in vivo as measured by positron emission tomography with a [¹¹C]-carfentanil ligand. In some embodiments, the μ-opioid receptor modulator is present in an amount that occupies at least about 95% of μ-opioid receptors in vivo as measured by positron emission tomography with a [¹¹C]-carfentanil ligand. In some embodiments, the μ-opioid receptor modulator is present in an amount that occupies at least about 96% of μ-opioid receptors in vivo as measured by positron emission tomography with a [¹¹C]-carfentanil ligand. In some embodiments, the μ-opioid receptor modulator is present in an amount that occupies at least about 97% of μ-opioid receptors in vivo as measured by positron emission tomography with a [¹¹C]-carfentanil ligand. In some embodiments, the μ-opioid receptor modulator is present in an amount that occupies at least about 98% of μ-opioid receptors in vivo as measured by positron emission tomography with a [¹¹C]-carfentanil ligand. In some embodiments, the μ-opioid receptor modulator is present in an amount that occupies at least about 99% of μ-opioid receptors in vivo as measured by positron emission tomography with a [¹¹C]-carfentanil ligand. In some embodiments, the μ-opioid receptor modulator is present in an amount that occupies at least about 99.1% of μ-opioid receptors in vivo as measured by positron emission tomography with a [¹¹C]-carfentanil ligand. In some embodiments, the μ-opioid receptor modulator is present in an amount that occupies at least about 99.2% of μ-opioid receptors in vivo as measured by positron emission tomography with a [¹¹C]-carfentanil ligand. In some embodiments, the μ-opioid receptor modulator is present in an amount that occupies at least about 99.3% of μ-opioid receptors in vivo as measured by positron emission tomography with a [¹¹C]-carfentanil ligand. In some embodiments, the μ-opioid receptor modulator is present in an amount that occupies at least about 99.4% of μ-opioid receptors in vivo as measured by positron emission tomography with a [¹¹C]-carfentanil ligand. In some embodiments, the μ-opioid receptor modulator is present in an amount that occupies at least about 99.5% of μ-opioid receptors in vivo as measured by positron emission tomography with a [¹¹C]-carfentanil ligand. In some embodiments, the μ-opioid receptor modulator is present in an amount that occupies at least about 99.6% of μ-opioid receptors in vivo as measured by positron emission tomography with a [¹¹C]-carfentanil ligand. In some embodiments, the μ-opioid receptor modulator is present in an amount that occupies at least about 99.7% of μ-opioid receptors in vivo as measured by positron emission tomography with a [¹¹C]-carfentanil ligand. In some embodiments, the μ-opioid receptor modulator is present in an amount that occupies at least about 99.8% of μ-opioid receptors in vivo as measured by positron emission tomography with a [¹¹C]-carfentanil ligand. In some embodiments, the μ-opioid receptor modulator is present in an amount that occupies at least about 99.9% of μ-opioid receptors in vivo as measured by positron emission tomography with a [¹¹C]-carfentanil ligand.

In some embodiments, the μ-opioid receptor modulator is present in an amount that occupies at least about 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99%, 99.1%, 99.2%, 99.3%, 99.4%, 99.5%, 99.6%, 99.7%, 99.8%, and/or 99.9% of μ-opioid receptors in vivo as measured by positron emission tomography with a [¹¹C]-carfentanil ligand.

In some embodiments, the μ-opioid receptor modulator is administered at a dosage that occupies at least 10% of μ-opioid receptors in the subject as measured by positron emission tomography with a [¹¹C]-carfentanil ligand. In some embodiments, the μ-opioid receptor modulator is administered at a dosage that occupies at least 25% of μ-opioid receptors in the subject. In some embodiments, the μ-opioid receptor modulator is administered at a dosage that occupies at least 50% of μ-opioid receptors in the subject. In some embodiments, the μ-opioid receptor modulator is administered at a dosage that occupies at least 75% of μ-opioid receptors in the subject. In some embodiments, the μ-opioid receptor modulator is administered at a dosage that occupies at least 90% of μ-opioid receptors in the subject. In some embodiments, the μ-opioid receptor modulator is administered at a dosage that occupies at least 99% of μ-opioid receptors in the subject.

In some embodiments, the μ-opioid receptor modulator prevents, ameliorates, and/or minimizes abuse of the NMDA receptor modulator by the subject. In some embodiments, the μ-opioid receptor modulator prevents, ameliorates, and/or minimizes abuse of the NMDA receptor modulator and an abused substance that is not the NMDA receptor modulator. In some embodiments, the abused substance is selected from the group consisting of alcohol, a stimulant, an opioid, cannabis, a hallucinogen, an inhalant, a sedative, a hypnotic, an anxiolytic, tobacco, caffeine, nicotine, and other (unknown) substances. In some embodiments, the stimulant comprises cocaine or amphetamine. In some embodiments, the hallucinogen comprises lysergic acid diethylamide (LSD) or phencyclidine. In some embodiments, the anxiolytic comprises barbiturate or benzodiazepine. In other embodiments, the subject has a comorbid addictive disease or disorder. In some embodiments, the administering has at least one effect selected from the group consisting of reduced anxiety, reduced irritability, reduced anger, and reduced alcohol consumption.

In some embodiments, the NMDA receptor modulator and μ-opioid receptor modulator are administered concurrently to the subject. In some embodiments, the NMDA receptor modulator and μ-opioid receptor modulator are administered separately to the subject. In some embodiments, the NMDA receptor modulator and μ-opioid receptor modulator are administered sequentially to the subject.

### Administration Dosage

The regimen of administration may affect what constitutes an effective amount. The therapeutic formulations may be administered to the subject either prior to or after the onset of a disease or disorder contemplated in the present application. Further, several divided dosages, as well as staggered dosages may be administered daily or sequentially, or the dose may be continuously infused, or may be a bolus injection. Further, the dosages of the therapeutic formulations may be proportionally increased or decreased as indicated by the exigencies of the therapeutic or prophylactic situation.

Administration of the compositions of the present application to a patient, preferably a mammal, more preferably a human, may be carried out using known procedures, at dosages and for periods of time effective to treat a disease or disorder contemplated in the present application. An effective amount of the therapeutic compound or composition necessary to achieve a therapeutic effect may vary according to factors such as the state of the disease or disorder in the patient: the age, sex, and weight of the patient; and the ability of the therapeutic compound to treat a disease or disorder contemplated in the present application. Dosage regimens may be adjusted to provide the optimum therapeutic response. For example, several divided doses may be administered daily or the dose may be proportionally reduced as indicated by the exigencies of the therapeutic situation. A non-limiting example of an effective dose range for a therapeutic compound of the present application is from about 1 and 5,000 mg/kg of body weight/per day. The pharmaceutical compositions useful for practicing the present application may be administered to deliver a dose of from ng/kg/day and 100 mg/kg/day. In some embodiments, the present application envisions administration of a dose which results in a concentration of the compound of the present application from 1 μM and 10 μM in a mammal. One of ordinary skill in the art would be able to study the relevant factors and make the determination regarding the effective amount of the therapeutic compound without undue experimentation.

Actual dosage levels of the active ingredients in the pharmaceutical compositions of this present application may be varied so as to obtain an amount of the active ingredient that is effective to achieve the desired therapeutic response for a particular patient, composition, and mode of administration, without being toxic to the patient.

In particular, the selected dosage level depends upon a variety of factors including the activity of the particular compound employed, the time of administration, the rate of excretion of the compound, the duration of the treatment, other drugs, compounds or materials used in combination with the compound, the age, sex, weight, condition, general health and prior medical history of the patient being treated, and like factors well, known in the medical arts.

A medical doctor, e.g., physician or veterinarian, having ordinary skill in the art may readily determine and prescribe the effective amount of the pharmaceutical composition required. For example, the physician or veterinarian could start doses of the compounds of the present application employed in the pharmaceutical composition at levels lower than that required in order to achieve the desired therapeutic effect and gradually increase the dosage until the desired effect is achieved.

In particular embodiments, it is especially advantageous to formulate the compound in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical vehicle. The dosage unit forms of the present application are dictated by and directly dependent on (a) the unique characteristics of the therapeutic compound and the particular therapeutic effect to be achieved, and (b) the limitations inherent in the art of compounding/formulating such a therapeutic compound for the treatment of a disease or disorder contemplated in the present application.

In some embodiments, the compositions of the present application are administered to the patient in dosages that range from one to five times per day or more. In another embodiment, the compositions of the present application are administered to the patient in range of dosages that include, but are not limited to, once every day, every two, days, every three days to once a week, and once every two weeks. It is readily apparent to one skilled in the art that the frequency of administration of the various combination compositions of the present application varies from individual to individual depending on many factors including, but not limited to, age, disease or disorder to be treated, gender, overall health, and other factors. Thus, the present application should not be construed to be limited to any particular dosage regime and the precise dosage and composition to be administered to any patient is determined by the attending physical taking all other factors about the patient into account.

Compounds of the present application for administration may be in the range of from about 1 μg to about 10,000 mg, about 20 μg to about 9,500 mg, about 40 μg to about 9,000 mg, about 75 μg to about 8,500 mg, about 150 μg to about 7,500 mg, about 200 μg to about 7,000 mg, about 3050 μg to about 6,000 mg, about 500 μg to about 5,000 mg, about 750 μg to about 4,000 mg, about 1 mg to about 3,000 mg, about 10 mg to about 2,500 mg, about 20 mg to about 2,000 mg, about 25 mg to about 1,500 mg, about 30 mg to about 1,000 mg, about 40 mg to about 900 mg, about 50 mg to about 800 mg, about 60 mg to about 750 mg, about 70 mg to about 600 mg, about 80 mg to about 500 mg, and any and all whole or partial increments therebetween.

In some embodiments, the dose of a compound of the present application is from about 1 mg and about 2,500 mg. In some embodiments, a dose of a compound of the present application used in compositions described herein is less than about 10,000 mg, or less than about 8,000 mg, or less than about 6,000 mg, or less than about 5,000 mg, or less than about 3,000 mg, or less than about 2,000 mg, or less than about 1,000 mg, or less than about 500 mg, or less than about 200 mg, or less than about 50 mg. Similarly, in some embodiments, a dose of a second compound as described herein is less than about 1,000 mg, or less than about 800 mg, or less than about 600 mg, or less than about 500 mg, or less than about 400 mg, or less than about 300 mg, or less than about 200 mg, or less than about 100 mg, or less than about 50 mg, or less than about 40 mg, or less than about 30 mg, or less than about 25 mg, or less than about 20 mg, or less than about 15 mg, or less than about 10 mg, or less than about 5 mg, or less than about 2 mg, or less than about 1 mg, or less than about 0.5 mg, and any and all whole or partial increments thereof.

The method comprises administering to the subject the μ-opioid receptor modulator in various dosages. In some embodiments, the μ-opioid receptor modulator is administered at a dosage of less than about 400 mg. In some embodiments, the μ-opioid receptor modulator is administered at a dosage of less than about 400 mg, less than about 350 mg, less than about 300 mg, less than about 250 mg, less than about 200 mg, less than about 150 mg, less than about 100 mg, less than about 90 mg, less than about 80 mg, less than about 70 mg, less than about 60 mg, less than about 50 mg, less than about 40 mg, less than about 30 mg, less than about 20 mg, less than about 10 mg or less than about 5 mg. In some embodiments, the μ-opioid receptor modulator is present in an amount of about 1 mg to about 5 mg, about 1 mg to about 10 mg, about 1 mg to about 20 mg, about 1 mg to about 50 mg, about 1 mg to about 80 mg, about 1 mg to about 100 mg, about 1 mg to about 150 mg, about 1 mg to about 200 mg, about 1 mg to about 250 mg, about 1 mg to about 300 mg, about 1 mg to about 350 mg, about 1 mg to about 400 mg, about 5 mg to about 10 mg, about 5 mg to about 20 mg, about 5 mg to about 50 mg, about 5 mg to about 80 mg, about 5 mg to about 100 mg, about 5 mg to about 150 mg, about 5 mg to about 200 mg, about 5 mg to about 250) mg, about 5 mg to about 300 mg, about 5 mg to about 350 mg, about 5 mg to about 400 mg, about 10 mg to about 20 mg, about 10 mg to about 50 mg, about 10 mg to about 80 mg, about 10 mg to about 100 mg, about 10 mg to about 150 mg, about 10 mg to about 200 mg, about 10 mg to about 250 mg, about 10 mg to about 300 mg, about 10 mg to about 350 mg, about 10 mg to about 400 mg, about 20 mg to about 50 mg, about 20 mg to about 80 mg, about 20 mg to about 100 mg, about 20 mg to about 150 mg, about 20 mg to about 200 mg, about 20 mg to about 250 mg, about 20 mg to about 300 mg, about 20 mg to about 350 mg, about 20 mg to about 400 mg, about 50 mg to about 80 mg, about 50 mg to about 100 mg, about 50) mg to about 150 mg, about 50 mg to about 200 mg, about 50 mg to about 250 mg, about 50) mg to about 300 mg, about 50 mg to about 350 mg, about 50 mg to about 400 mg, about 80) mg to about 100 mg, about 80 mg to about 150 mg, about 80 mg to about 200 mg, about 80 mg to about 250 mg, about 80 mg to about 300 mg, about 80 mg to about 350 mg, about 80 mg to about 400 mg, about 100 mg to about 150 mg, about 100 mg to about 200 mg, about 100 mg to about 250 mg, about 100 mg to about 300 mg, about 100 mg to about 350 mg, about 100 mg to about 400 mg, about 150 mg to about 200 mg, about 150 mg to about 250 mg, about 150 mg to about 300 mg, about 150 mg to about 350 mg, about 150 mg to about 400 mg, about 200 mg to about 250 mg, about 200 mg to about 300 mg, about 200 mg to about 350 mg, about 200 mg to about 400 mg, about 250 mg to about 300 mg, about 250 mg to about 350 mg, about 250 mg to about 400 mg, about 300 mg to about 350 mg, or about 350 mg to about 400 mg.

The method comprises administering to the subject the NMDA receptor modulator in various dosages. In some embodiments, the NMDA receptor modulator is administered at a dosage of less than about 150 mg. In some embodiments, the NMDA receptor modulator is administered at a dosage of less than about 150 mg, less than about 100 mg, less than about 90 mg, less than about 80 mg, less than about 70 mg, less than about 60 mg, less than about 50 mg, less than about 40 mg, less than about 30 mg, less than about 20 mg, less than about 10 mg or less than about 5 mg. In some embodiments, the NMDA receptor modulator is administered at a dosage of about 1 mg to about 5 mg, about 1 mg to about 10 mg, about 1 mg to about 20 mg, about 1 mg to about 50 mg, about 1 mg to about 40 mg, about 1 mg to about 50 mg, about 1 mg to about 60 mg, about 1 mg to about 70 mg, about 1 mg to about 80 mg, about 1 mg to about 90 mg, about 1 mg to about 100 mg, about 1 mg to about 150 mg, about 5 mg to about 10 mg, about 5 mg to about 20 mg, about 5 mg to about 50 mg, about 5 mg to about 40 mg, about 5 mg to about 50 mg, about 5 mg to about 60 mg, about 5 mg to about 70 mg, about 5 mg to about 80 mg, about 5 mg to about 90 mg, about 5 mg to about 100 mg, about 5 mg to about 150 mg, about 10 mg to about 20 mg, about 10 mg to about 50 mg, about 10 mg to about 40 mg, about 10 mg to about 50 mg, about 10 mg to about 60 mg, about 10 mg to about 70 mg, about 10 mg to about 80 mg, about 10 mg to about 90 mg, about 10 mg to about 100 mg, about 10 mg to about 150 mg, about 20 mg to about 50 mg, about 20 mg to about 40 mg, about 20 mg to about 50 mg, about 20 mg to about 60 mg, about 20 mg to about 70 mg, about 20 mg to about 80 mg, about 20 mg to about 90 mg, about 20 mg to about 100 mg, about 20 mg to about 150 mg, about 30 mg to about 40 mg, about 30 mg to about 50 mg, about 30 mg to about 60 mg, about 30 mg to about 70 mg, about 30 mg to about 80 mg, about 30 mg to about 90 mg, about 30 mg to about 100 mg, about 30 mg to about 150 mg, about 40 mg to about 50 mg, about 40 mg to about 60 mg, about 40 mg to about 70 mg, about 40 mg to about 80 mg, about 40 mg to about 90 mg, about 40 mg to about 100 mg, about 40 mg to about 150 mg, about 50 mg to about 60 mg, about 50 mg to about 70 mg, about 50 mg to about 80 mg, about 50 mg to about 90 mg, about 50 mg to about 100 mg, about 50 mg to about 150 mg, about 60 mg to about 70 mg, about 60 mg to about 80 mg, about 60 mg to about 90 mg, about 60 mg to about 100 mg, about 60 mg to about 150 mg, about 70 mg to about 80 mg, about 70 mg to about 90 mg, about 70 mg to about 100 mg, about 70 mg to about 150 mg, about 80 mg to about 90 mg, about 80 mg to about 100 mg, about 80 mg to about 150 mg, about 90 mg to about 100 mg, about 90 mg to about 150 mg, or about 100 mg to about 150 mg.

Table 1 and Table 2 demonstrates possible formulations of ketamine and naltrexone.

TABLE 1
Intramuscular (IM) coformulation of ketamine and naltrexone
The following doses of intramuscular coformulation of ketamine (first dose) and naltrexone
(second dose) can be used (ketamine dose [mg] + naltrexone dose [mg]).

| IM Dose of | | | | | | | | | | | |
|---|---|---|---|---|---|---|---|---|---|---|---|
| Naltrexone | | | | | | | | | | | |
| (between 1 mg | IM Dose of Ketamine (between 10 mg and 60 mg; about 0.25 mg/kg-1.0 mg/kg) | | | | | | | | | | |
| and 100 mg) | 10 mg | 15 mg | 20 mg | 25 mg | 30 mg | 35 mg | 40 mg | 45 mg | 50 mg | 55 mg | 60 mg |
| 5 mg | 10 + 5 | 15 + 5 | 20 + 5 | 25 + 5 | 30 + 5 | 35 + 5 | 40 + 5 | 45 + 5 | 50 + 5 | 55 + 5 | 60 + 5 |
| 10 mg | 10 + 10 | 15 + 10 | 20 + 10 | 25 + 10 | 30 + 10 | 35 + 10 | 40 + 10 | 45 + 10 | 50 + 10 | 55 + 10 | 60 + 10 |
| 15 mg | 10 + 15 | 15 + 15 | 20 + 15 | 25 + 15 | 30 + 15 | 35 + 15 | 40 + 15 | 45 + 15 | 50 + 15 | 55 + 15 | 60 + 15 |
| 20 mg | 10 + 20 | 15 + 20 | 20 + 20 | 25 + 20 | 30 + 20 | 35 + 20 | 40 + 20 | 45 + 20 | 50 + 20 | 55 + 20 | 60 + 20 |
| 25 mg | 10 + 25 | 15 + 25 | 20 + 25 | 25 + 25 | 30 + 25 | 35 + 25 | 40 + 25 | 45 + 25 | 50 + 25 | 55 + 25 | 60 + 25 |
| 30 mg | 10 + 30 | 15 + 30 | 20 + 30 | 25 + 30 | 30 + 30 | 35 + 30 | 40 + 30 | 45 + 30 | 50 + 30 | 55 + 30 | 60 + 30 |
| 35 mg | 10 + 35 | 15 + 35 | 20 + 35 | 25 + 35 | 30 + 35 | 35 + 35 | 40 + 35 | 45 + 35 | 50 + 35 | 55 + 35 | 60 + 35 |
| 40 mg | 10 + 40 | 15 + 40 | 20 + 40 | 25 + 40 | 30 + 40 | 35 + 40 | 40 + 40 | 45 + 40 | 50 + 40 | 55 + 40 | 60 + 40 |
| 45 mg | 10 + 45 | 15 + 45 | 20 + 45 | 25 + 45 | 30 + 45 | 35 + 45 | 40 + 45 | 45 + 45 | 50 + 45 | 55 + 45 | 60 + 45 |
| 50 mg | 10 + 50 | 15 + 50 | 20 + 50 | 25 + 50 | 30 + 50 | 35 + 50 | 40 + 50 | 45 + 50 | 50 + 50 | 55 + 50 | 60 + 50 |
| 55 mg | 10 + 55 | 15 + 55 | 20 + 55 | 25 + 55 | 30 + 55 | 35 + 55 | 40 + 55 | 45 + 55 | 50 + 55 | 55 + 55 | 60 + 55 |
| 60 mg | 10 + 60 | 15 + 60 | 20 + 60 | 25 + 60 | 30 + 60 | 35 + 60 | 40 + 60 | 45 + 60 | 50 + 60 | 55 + 60 | 60 + 60 |
| 65 mg | 10 + 65 | 15 + 65 | 20 + 65 | 25 + 65 | 30 + 65 | 35 + 65 | 40 + 65 | 45 + 65 | 50 + 65 | 55 + 65 | 60 + 65 |
| 70 mg | 10 + 70 | 15 + 70 | 20 + 70 | 25 + 70 | 30 + 70 | 35 + 70 | 40 + 70 | 45 + 70 | 50 + 70 | 55 + 70 | 60 + 70 |
| 75 mg | 10 + 75 | 15 + 75 | 20 + 75 | 25 + 75 | 30 + 75 | 35 + 75 | 40 + 75 | 45 + 75 | 50 + 75 | 55 + 75 | 60 + 75 |
| 80 mg | 10 + 80 | 15 + 80 | 20 + 80 | 25 + 80 | 30 + 80 | 35 + 80 | 40 + 80 | 45 + 80 | 50 + 80 | 55 + 80 | 60 + 80 |
| 85 mg | 10 + 85 | 15 + 85 | 20 + 85 | 25 + 85 | 30 + 85 | 35 + 85 | 40 + 85 | 45 + 85 | 50 + 85 | 55 + 85 | 60 + 85 |
| 90 mg | 10 + 90 | 15 + 90 | 20 + 90 | 25 + 90 | 30 + 90 | 35 + 90 | 40 + 90 | 45 + 90 | 50 + 90 | 55 + 90 | 60 + 90 |
| 95 mg | 10 + 95 | 15 + 95 | 20 + 95 | 25 + 95 | 30 + 95 | 35 + 95 | 40 + 95 | 45 + 95 | 50 + 95 | 55 + 95 | 60 + 95 |
| 100 mg | 10 + 100 | 15 + 100 | 20 + 100 | 25 + 100 | 30 + 100 | 35 + 100 | 40 + 100 | 45 + 100 | 50 + 100 | 55 + 100 | 60 + 100 |

TABLE 2
Intranasal (IN) coformulation of ketamine and naltrexone
The following doses of intranasal coformulation of ketamine (first dose) and naltrexone
(second dose) can be used (ketamine dose [mg] + naltrexone dose [mg]).

| IN | | | | | | | | | | | | | | | | | |
|---|---|---|---|---|---|---|---|---|---|---|---|---|---|---|---|---|---|
| Dose | | | | | | | | | | | | | | | | | |
| of | | | | | | | | | | | | | | | | | |
| Naltre- | | | | | | | | | | | | | | | | | |
| xone | IN Dose of Ketamine (between 15 mg and 95 mg) | | | | | | | | | | | | | | | | |
| (1 mg- | 15 | 20 | 25 | 30 | 35 | 40 | 45 | 50 | 55 | 60 | 65 | 70 | 75 | 80 | 85 | 90 | 95 |
| 90 mg) | mg | mg | mg | mg | mg | mg | mg | mg | mg | mg | mg | mg | mg | mg | mg | mg | mg |
| 1 mg | 15 + 1 | 20 + 1 | 25 + 1 | 30 + 1 | 35 + 1 | 40 + 1 | 45 + 1 | 50 + 1 | 55 + 1 | 60 + 1 | 65 + 1 | 70 + 1 | 75 + 1 | 80 + 1 | 85 + 1 | 90 + 1 | 95 + 1 |
| 5 mg | 15 + 5 | 20 + 5 | 25 + 5 | 30 + 5 | 35 + 5 | 40 + 5 | 45 + 5 | 50 + 5 | 55 + 5 | 60 + 5 | 65 + 5 | 70 + 5 | 75 + 5 | 80 + 5 | 85 + 5 | 90 + 5 | 95 + 5 |
| 10 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 10 | 10 | 10 | 10 | 10 | 10 | 10 | 10 | 10 | 10 | 10 | 10 | 10 | 10 | 10 | 10 | 10 |
| 15 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 15 | 15 | 15 | 15 | 15 | 15 | 15 | 15 | 15 | 15 | 15 | 15 | 15 | 15 | 15 | 15 | 15 |
| 20 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 20 | 20 | 20 | 20 | 20 | 20 | 20 | 20 | 20 | 20 | 20 | 20 | 20 | 20 | 20 | 20 | 20 |
| 25 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 25 | 25 | 25 | 25 | 25 | 25 | 25 | 25 | 25 | 25 | 25 | 25 | 25 | 25 | 25 | 25 | 25 |
| 30 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 30 | 30 | 30 | 30 | 30 | 30 | 30 | 30 | 30 | 30 | 30 | 30 | 30 | 30 | 30 | 30 | 30 |
| 35 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 35 | 35 | 35 | 35 | 35 | 35 | 35 | 35 | 35 | 35 | 35 | 35 | 35 | 35 | 35 | 35 | 35 |
| 40 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 40 | 40 | 40 | 40 | 40 | 40 | 40 | 40 | 40 | 40 | 40 | 40 | 40 | 40 | 40 | 40 | 40 |
| 45 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 45 | 45 | 45 | 45 | 45 | 45 | 45 | 45 | 45 | 45 | 45 | 45 | 45 | 45 | 45 | 45 | 45 |
| 50 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 50 | 50 | 50 | 50 | 50 | 50 | 50 | 50 | 50 | 50 | 50 | 50 | 50 | 50 | 50 | 50 | 50 |
| 55 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 55 | 55 | 55 | 55 | 55 | 55 | 55 | 55 | 55 | 55 | 55 | 55 | 55 | 55 | 55 | 55 | 55 |
| 60 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 60 | 60 | 60 | 60 | 60 | 60 | 60 | 60 | 60 | 60 | 60 | 60 | 60 | 60 | 60 | 60 | 60 |
| 65 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 65 | 65 | 65 | 65 | 65 | 65 | 65 | 65 | 65 | 65 | 65 | 65 | 65 | 65 | 65 | 65 | 65 |
| 70 mg | 15 + | 20 + | 25 + | 30 + | 35 + | 40- | 45 + | 50 + | 55 + | 60 + | 65 + | 70 + | 75 + | 80 + | 85 + | 90 + | 95 + |
| | 70 | 70 | 70 | 70 | 70 | 70 | 70 | 70 | 70 | 70 | 70 | 70 | 70 | 70 | 70 | 70 | 70 |
| 75 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 75 | 75 | 75 | 75 | 75 | 75 | 75 | 75 | 75 | 75 | 75 | 75 | 75 | 75 | 75 | 75 | 75 |
| 80 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 80 | 80 | 80 | 80 | 80 | 80 | 80 | 80 | 80 | 80 | 80 | 80 | 80 | 80 | 80 | 80 | 80 |
| 85 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 85 | 85 | 85 | 85 | 85 | 85 | 85 | 85 | 85 | 85 | 85 | 85 | 85 | 85 | 85 | 85 | 85 |
| 90 mg | 15 + | 20 + | 25 + | 30 + | 35 + | 40 + | 45 + | 50 + | 55 + | 60 + | 65 + | 70 + | 75 + | 80 + | 85 + | 90 + | 95 + |
| | 90 | 90 | 90 | 90 | 90 | 90 | 90 | 90 | 90 | 90 | 90 | 90 | 90 | 90 | 90 | 90 | 90 |

### Administration Frequency

In some embodiments, the NMDA receptor modulator is administered once per day, twice per day, three times per day, once per week, twice per week, three times per week, once per month, twice per month, or three times per month.

In some embodiments, the μ-opioid receptor modulator is administered once per day, twice per day, three times per day, once per week, twice per week, three times per week, once per month, twice per month or three times per month.

### Administration Formulations

In certain embodiments, the compositions of the present application are formulated using one or more pharmaceutically acceptable excipients or carriers. In certain embodiments, the pharmaceutical compositions of the present application comprise a therapeutically effective amount of a compound of the present application and a pharmaceutically acceptable carrier.

The carrier may be a solvent or dispersion medium containing, for example, water, ethanol, polyol (for example, glycerol, propylene glycol, and liquid polyethylene glycol, and the like), suitable mixtures thereof, and vegetable oils. The proper fluidity may be maintained, for example, by the use of a coating such as lecithin, by the maintenance of the required particle size in the case of dispersion and by the use of surfactants. Prevention of the action of microorganisms may be achieved by various antibacterial and antifungal agents, for example, parabens, chlorobutanol, phenol, ascorbic acid, thimerosal, and the like. In many cases, it is preferable to include isotonic agents, for example, sugars, sodium chloride, or polyalcohols such as mannitol and sorbitol, in the composition. Prolonged absorption of the injectable compositions may be brought about by including in the composition an agent which delays absorption, for example, aluminum monostearate or gelatin.

In certain embodiments, the present application is directed to a packaged pharmaceutical composition comprising a container holding a therapeutically effective amount of a compound of the present application, alone or in combination with a second pharmaceutical agent; and instructions for using the compound to treat, prevent, or reduce one or more symptoms of a disease or disorder contemplated in the present application.

Formulations may be employed in admixtures with conventional excipients, i.e., pharmaceutically acceptable organic or inorganic carrier substances suitable for oral, parenteral, nasal, intravenous, subcutaneous, enteral, or any other suitable mode of administration, known to the art. The pharmaceutical preparations may be sterilized and if desired mixed with auxiliary agents, e.g., lubricants, preservatives, stabilizers, wetting agents, emulsifiers, salts for influencing osmotic pressure buffers, coloring, flavoring and/or aromatic substances and the like. They may also be combined where desired with other active agents, e.g., anti-AD agents.

Suitable compositions and dosage forms include, for example, tablets, capsules, caplets, pills, gel caps, troches, dispersions, suspensions, solutions, syrups, granules, beads, transdermal patches, gels, powders, pellets, magmas, lozenges, creams, pastes, plasters, lotions, discs, suppositories, liquid sprays for nasal or oral administration, dry powder or aerosolized formulations for inhalation, compositions and formulations for intravesical administration and the like. It should be understood that the formulations and compositions that would be useful in the present application are not limited to the particular formulations and compositions that are described herein.

### Administration Routes

Routes of administration of any of the compositions of the present application include oral, nasal, rectal, intravaginal, parenteral, buccal, sublingual or topical. The compounds for use in the present application may be formulated for administration by any suitable route, such as for oral or parenteral, for example, transdermal, transmucosal (e.g., sublingual, lingual, (trans) buccal, (trans) urethral, vaginal (e.g., trans- and perivaginally), (intra) nasal and (trans) rectal), intravesical, intrapulmonary, intraduodenal, intragastrical, intrathecal, subcutaneous, intramuscular, intradermal, intra-arterial, intravenous, intrabronchial, inhalation, and topical administration.

In some embodiments, the NMDA receptor modulator and μ-opioid receptor modulator are administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

In some embodiments, the pharmaceutical composition is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

### Intranasal Administration

One may use intranasal administration of the present disclosure, such as with nasal solutions or sprays, aerosols or inhalants. Nasal solutions are usually aqueous solutions designed to be administered to the nasal passages in drops or sprays. Nasal solutions are prepared so that they are similar in many respects to nasal secretions. Thus, the aqueous nasal solutions usually are isotonic and slightly buffered to maintain a pH of 5.5 to 6.5. In addition, antimicrobial preservatives, similar to those used in ophthalmic preparations and appropriate drug stabilizers, if required, may be included in the formulation. Various commercial nasal preparations are known and include, for example, antibiotics and antihistamines.

### Oral Administration

For oral application, particularly suitable are tablets, dragees, liquids, drops, suppositories, or capsules, caplets and gelcaps. The compositions intended for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose: granulating and disintegrating agents such as cornstarch: binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the present application may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulfate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

The present application also includes a multi-layer tablet comprising a layer providing for the delayed release of one or more compounds of the present application, and a further layer providing for the immediate release of a medication for treatment of a disease or disorder contemplated in the present application. Using a wax/pH-sensitive polymer mix, a gastric insoluble composition may be obtained in which the active ingredient is entrapped, ensuring its delayed release.

### Parenteral Administration

As used herein, “parenteral administration” of a pharmaceutical composition includes any route of administration characterized by physical breaching of a tissue of a subject and administration of the pharmaceutical composition through the breach in the tissue. Parenteral administration thus includes, but is not limited to, administration of a pharmaceutical composition by injection of the composition, by application of the composition through a surgical incision, by application of the composition through a tissue-penetrating non-surgical wound, and the like. In particular, parenteral administration is contemplated to include, but is not limited to, subcutaneous, intravenous, intraperitoneal, intramuscular, intrasternal injection, and kidney dialytic infusion techniques.

Formulations of a pharmaceutical composition suitable for parenteral administration comprise the active ingredient combined with a pharmaceutically acceptable carrier, such as sterile water or sterile isotonic saline. Such formulations may be prepared, packaged, or sold in a form suitable for bolus administration or for continuous administration. Injectable formulations may be prepared, packaged, or sold in unit dosage form, such as in ampules or in multidose containers containing a preservative. Formulations for parenteral administration include, but are not limited to, suspensions, solutions, emulsions in oily or aqueous vehicles, pastes, and implantable sustained-release or biodegradable formulations. Such formulations may further comprise one or more additional ingredients including, but not limited to, suspending, stabilizing, or dispersing agents. In certain embodiments of a formulation for parenteral administration, the active ingredient is provided in dry (i.e., powder or granular) form for reconstitution with a suitable vehicle (e.g., sterile pyrogen free water) prior to parenteral administration of the reconstituted composition.

The pharmaceutical compositions may be prepared, packaged, or sold in the form of a sterile injectable aqueous or oily suspension or solution. This suspension or solution may be formulated according to the known art, and may comprise, in addition to the active ingredient, additional ingredients such as the dispersing agents, wetting agents, or suspending agents described herein. Such sterile injectable formulations may be prepared using a non-toxic parenterally-acceptable diluent or solvent, such as water or 1,3-butanediol, for example. Other acceptable diluents and solvents include, but are not limited to, Ringer's solution, isotonic sodium chloride solution, and fixed oils such as synthetic mono- or di-glycerides. Other parentally-administrable formulations which are useful include those which comprise the active ingredient in microcrystalline form, in a liposomal preparation, or as a component of a biodegradable polymer system. Compositions for sustained release or implantation may comprise pharmaceutically acceptable polymeric or hydrophobic materials such as an emulsion, an ion exchange resin, a sparingly soluble polymer, or a sparingly soluble salt.

### Intramuscular Administration

An intramuscular injection is a technique used to deliver a medication deep into the muscles. This allows the medication to be absorbed into the bloodstream quickly. Intramuscular injections are used when other types of delivery methods are not recommended. Intramuscular injections may be used instead of intravenous injections because some drugs are irritating to veins, or because a suitable vein cannot be located. It may be used instead of oral delivery because some drugs are destroyed by the digestive system when a drug is swallowed. Intramuscular injections are absorbed faster than subcutaneous injections. This is because muscle tissue has a greater blood supply than the tissue just under the skin. Muscle tissue can also hold a larger volume of medication than subcutaneous tissue.

### Subcutaneous Administration

For subcutaneous administration, a short needle is used to inject a drug into the tissue layer between the skin and the muscle. Medication given this way is usually absorbed more slowly than if injected into a vein, sometimes over a period of 24 hours. Subcutaneous administration is used when other methods of administration might be less effective. For example, some medications cannot be given by mouth because acid and enzymes in the stomach would destroy them. Other methods, like intravenous injection, can be difficult and costly. For small amounts of delicate drugs, a subcutaneous injection can be a useful, safe, and convenient method.

### Controlled Release Formulations and Drug Delivery Systems

In certain embodiments, the formulations of the present application may be, but are not limited to, short-term, rapid-offset, as well as controlled, for example, sustained release, delayed release and pulsatile release formulations.

The term sustained release is used in its conventional sense to refer to a drug formulation that provides for gradual release of a drug over an extended period of time, and that may, although not necessarily, result in substantially constant blood levels of a drug over an extended time period. The period of time may be as long as a month or more and should be a release which is longer that the same amount of agent administered in bolus form.

For sustained release, the compounds may be formulated with a suitable polymer or hydrophobic material that provides sustained release properties to the compounds. As such, the compounds for use the method of the present application may be administered in the form of microparticles, for example, by injection or in the form of wafers or discs by implantation.

In certain embodiments of the present application, the compounds of the present application are administered to a patient, alone or in combination with another pharmaceutical agent, using a sustained release formulation.

The term delayed release is used herein in its conventional sense to refer to a drug formulation that provides for an initial release of the drug after some delay following drug administration and that may, although not necessarily, includes a delay of from about 10 minutes up to about 12 hours.

The term pulsatile release is used herein in its conventional sense to refer to a drug formulation that provides release of the drug in such a way as to produce pulsed plasma profiles of the drug after drug administration.

The term immediate release is used in its conventional sense to refer to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Administration of Coformulation

In certain embodiments, the method for treating, ameliorating, and/or preventing a disease or disorder in a subject in need thereof comprises administering a coformulation to the subject. The coformulation comprises a N-methyl-D-aspartate receptor modulator and a μ-opioid receptor modulator and is repeatedly administered to the subject.

In certain embodiments, the N-methyl-D-aspartate receptor modulator is a N-methyl-D-aspartate receptor antagonist. In certain embodiments, the N-methyl-D-aspartate receptor modulator is a N-methyl-D-aspartate receptor negative allosteric modulator. In certain embodiments, the N-methyl-D-aspartate receptor modulator is a N-methyl-D-aspartate receptor partial agonist. In some embodiments, N-methyl-D-aspartate receptor modulator comprises a compound selected from the group consisting of ketamine, R-ketamine, S-ketamine, nitrous oxide, memantine, amantadine, racemic dextromethorphan, dextromethorphan, lanicemine, phencyclidine, dizocilpine, CERC-301, CGP 37849, 1-aminocylopropanecarboxylic acid, traxoprodil, Ro 25-6981, eliprodil, methoxetamine, CPPene, AP5, AP7, Selfotel (CGS-19755), minocycline, nitromemantine, PD-137889, rolicyclidine, tenocyclidine, methoxydine, tiletamine, neramexane, etoxadrol, dexoxadrol, WMS-2539, NEFA, remacemide, 3-MeO-PCP, 8A-PDHQ, atomoxetine, AZD6765, agmatine, chloroform, delucemine, dextrallorphan, dextrorphan, diphenidine, eticyclidine, gacyclidine, aptiganel, HU-211, huperzine A, dipeptide D-Phe-L-Tyr, ibogaine, rhynchophylline, rapastinel, NRX-1074, 7-Chlorokynurenic acid, 4-Chlorokynurenine, 5,7-Dichlorokynurenic acid, Kynurenic acid, TK-40, L-Phenylalanine, xenon, methadone, EU1180-438, radiprodil, Ifenprodil, TCN-201, MPX-004, MPX-007, NAB-14, EVT-101, QNZ-46, DQP-1105, pregnanolone sulfate (3α5βS), UBP608, UBP618, UBP551, UBP512, HA-966, felbamate, PEAQX (NVP-AAM077), PD0196860, RGH896, MK0657, L701324, LY293558, LY300164, LY246492, LY202157, NYX-783, and/or any salt, solvate, enantiomer, tautomer, stereoisomer, and/or geometric isomer thereof, and/or any mixtures thereof. In another embodiments, the N-methyl-D-aspartate receptor modulator comprises ketamine, and/or any salt, solvate, enantiomer, tautomer, stereoisomer, and/or geometric isomer thereof, and/or any mixtures thereof.

In some embodiments, the N-methyl-D-aspartate receptor modulator is present in an amount of about 10 mg to about 60 mg. In some embodiments, the N-methyl-D-aspartate receptor modulator is present in an amount of about 15 mg to about 95 mg. In some embodiments, the N-methyl-D-aspartate receptor modulator is present in an amount of about 50 mg. In some embodiments, the N-methyl-D-aspartate receptor modulator is present in an amount of about 10 mg, about 15 mg, about 20 mg, about 25 mg, about 30 mg, about 35 mg, about 40 mg, about 45 mg, about 50 mg, about 55 mg, about 60 mg, about 65 mg, about 70) mg, about 75 mg, about 80 mg, about 85 mg, about 90 mg, or 95 mg. In some embodiments, the N-methyl-D-aspartate receptor modulator is present in an amount of between about 10 mg to about 95 mg, between about 15 mg to about 90 mg, between about 20 mg to about 85 mg, between about 25 mg to about 80 mg, between about 30 mg to about 75 mg, between about 35 mg to about 70 mg, between about 40 mg to about 65 mg, between about 45 mg to about 60 mg, or between about 50 mg to about 55 mg.

In certain embodiments, the μ-opioid receptor modulator is a μ-opioid receptor antagonist. In certain embodiments, the μ-opioid receptor modulator is a μ-opioid receptor negative allosteric modulator. In certain embodiments, the μ-opioid receptor modulator is a μ-opioid receptor partial agonist. In some embodiments, the μ-opioid receptor modulator comprises a compound selected from the group consisting of naltrexone, naloxone, nalmefene, nalodeine, and/or any salt, solvate, enantiomer, tautomer, stereoisomer, and/or geometric isomer thereof, and/or any mixtures thereof. In another embodiments, the μ-opioid receptor modulator comprises naltrexone, and/or any salt, solvate, enantiomer, tautomer, stereoisomer, and/or geometric isomer thereof, and/or any mixtures thereof.

In some embodiments, the μ-opioid receptor modulator is present in an amount of lower than about 400 mg. In some embodiments, the μ-opioid receptor modulator is present in an amount of lower than about 400 mg, lower than about 350 mg, lower than about 300 mg, lower than about 250 mg, lower than about 200 mg, lower than about 150 mg, lower than about 100 mg, lower than about 50 mg, lower than about 10 mg, or lower than about 5 mg, or lower than about 1 mg.

In some embodiments, the μ-opioid receptor modulator is present in an amount of about 300 mg to about 400 mg. In some embodiments, the μ-opioid receptor modulator is present in an amount of about 200 mg to about 300 mg. In some embodiments, the μ-opioid receptor modulator is present in an amount of about 100 mg to about 200 mg. In some embodiments, the μ-opioid receptor modulator is present in an amount of about 5 mg to about 100 mg. In some embodiments, the μ-opioid receptor modulator is present in an amount of about 1 mg to about 90 mg. In some embodiments, the μ-opioid receptor modulator is present in an amount of about 2 mg to about 10 mg. In some embodiments, the μ-opioid receptor modulator is present in an amount of about 1 mg to about 400 mg, about 2 mg to 350 mg, about 5 mg to about 300 mg, about 10 mg to 250 mg, about 20 mg to about 200 mg, about 40 mg to 150 mg, about 60 mg to about 100 mg, or about 80 mg to 90 mg.

In some embodiments, the coformulation disclosed herein may be administered once per day, twice per day, or three times per day. In some embodiments, the coformulation disclosed herein may be administered once per week, twice per week, or three times per week. In some embodiments, the coformulation disclosed herein may be administered once per month, twice per month, or three times per month.

In some embodiments, the coformulation disclosed herein may be administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

In some embodiments, the coformulation of the present application are useful for treating, ameliorating, and/or preventing a disease or disorder that is at least one selected from the group consisting of major depressive disorder, major depressive episode in bipolar disorder (bipolar depression), bipolar I disorder, bipolar II disorder, persistent depressive disorder (dysthymia), disruptive mood dysregulation disorder, major depressive disorder (including major depressive episode), premenstrual dysphoric disorder, substance/medication-induced depressive disorder, depressive disorder due to another medical condition, other specified depressive disorder, unspecified depressive disorder, anxiety disorder, generalized anxiety disorder, social anxiety disorder (social phobia), specific phobia, panic disorder, agoraphobia, separation anxiety disorder, selective mutism, substance-induced anxiety disorder, medication-induced anxiety disorder, anxiety disorder due to another medical condition, borderline personality disorder, treatment-resistant depression, unspecified anxiety disorder, and posttraumatic stress disorder. In another embodiments, the subject may suffer from a comorbid substance use disorder.

In certain embodiments, presence of the μ-opioid receptor modulator disclosed herein may prevent, ameliorate, and/or minimize abuse of the N-methyl-D-aspartate receptor modulator and an abused substance that is not the N-methyl-D-aspartate receptor modulator. In some embodiments, the abused substance is selected from the group consisting of alcohol, a stimulant, an opioid, cannabis, a hallucinogen, an inhalant, a sedative, a hypnotic, an anxiolytic, tobacco, caffeine, nicotine, and other (unknown) substances. In some embodiments, the stimulant comprises cocaine and/or amphetamine. In some embodiments, the hallucinogen comprises lysergic acid diethylamide (LSD) and/or phencyclidine. In some embodiments, the anxiolytic comprises a barbiturate and/or a benzodiazepine. In some embodiments, administering has at least one effect selected from the group consisting of reduced anxiety, reduced irritability, reduced anger, and/or reduced alcohol consumption.

### Combination Therapies

In some embodiments, the compositions of the present application are useful for treating or preventing a disease or disorder contemplated herein in a mammal in need thereof in combination with at least one additional agents. This additional agent may comprise compounds identified herein or compounds, e.g., commercially available compounds, known to treat, prevent or reduce the symptoms of a disease or disorder contemplated herein.

In some embodiments, the at least one additional compound useful for treating or preventing depression comprises citalopram. In some embodiments, the at least one additional compound useful for treating or preventing depression comprises escitalopram. In some embodiments, the at least one additional compound useful for treating or preventing depression comprises paroxetine. In some embodiments, the at least one additional compound useful for treating or preventing depression comprises fluoxetine. In some embodiments, the at least one additional compound useful for treating or preventing depression comprises sertraline. In some embodiments, the at least one additional compound useful for treating or preventing depression comprises femoxetine. In some embodiments, the at least one additional compound useful for treating or preventing depression comprises tranylcypromine. In some embodiments, the at least one additional compound useful for treating or preventing depression comprises selegiline. In some embodiments, the at least one additional compound useful for treating or preventing depression comprises isocarboxzaid. In some embodiments, the at least one additional compound useful for treating or preventing depression comprises phenelzine. In some embodiments, the at least one additional compound useful for treating or preventing depression comprises fluvoxamine. In some embodiments, the at least one additional compound useful for treating or preventing depression comprises trimipramine. In some embodiments, the at least one additional compound useful for treating or preventing depression comprises amitriptyline. In some embodiments, the at least one additional compound useful for treating or preventing depression comprises nortriptyline. In some embodiments, the at least one additional compound useful for treating or preventing depression comprises desipramine. In some embodiments, the at least one additional compound useful for treating or preventing depression comprises protriptyline. In some embodiments, the at least one additional compound useful for treating or preventing depression comprises imipramine. In some embodiments, the at least one additional compound useful for treating or preventing depression comprises doxepin. In some embodiments, the at least one additional compound useful for treating or preventing depression comprises clominpramine. In some embodiments, the at least one additional compound useful for treating or preventing depression comprises maprotiline. In some embodiments, the at least one additional compound useful for treating or preventing depression comprises bupropion. In some embodiments, the at least one additional compound useful for treating or preventing depression comprises nefazodone. In some embodiments, the at least one additional compound useful for treating or preventing depression comprises trazodone. In some embodiments, the at least one additional compound useful for treating or preventing depression comprises venlafaxine. In some embodiments, the at least one additional compound useful for treating or preventing depression comprises duloxetine. In some embodiments, the at least one additional compound useful for treating or preventing depression comprises desvenlafaxine. In some embodiments, the at least one additional compound useful for treating or preventing depression comprises mirtazapine. In some embodiments, the at least one additional compound useful for treating or preventing depression comprises milnacipran. In some embodiments, the at least one additional compound useful for treating or preventing depression comprises any at least one additional compound disclosed herein, and/or any salt, solvate, enantiomer, tautomer and geometric isomer thereof, or any mixtures thereof.

In other embodiments, the at least one additional compound comprises rapamycin (Sirolimus). In other embodiments, the at least one additional compound comprises BEZ235. In other embodiments, the at least one additional compound comprises everolimus. In other embodiments, the at least one additional compound comprises AZD8055. In other embodiments, the at least one additional compound comprises AZD-2014. In other embodiments, the at least one additional compound comprises Temsirolimus. In other embodiments, the at least one additional compound comprises KU-0063794. In other embodiments, the at least one additional compound comprises PI-103. In other embodiments, the at least one additional compound comprises Torkinib. In other embodiments, the at least one additional compound comprises Tacrolimus. In other embodiments, the at least one additional compound comprises Ridaforolimus. In other embodiments, the at least one additional compound comprises INK-128. In other embodiments, the at least one additional compound comprises Voxtalisib. In other embodiments, the at least one additional compound comprises Torin-1. In other embodiments, the at least one additional compound comprises Torin-2. In other embodiments, the at least one additional compound comprises Omipalisib. In other embodiments, the at least one additional compound comprises OSI-027. In other embodiments, the at least one additional compound comprises Apitolisib. In other embodiments, the at least one additional compound comprises GSK1059615. In other embodiments, the at least one additional compound comprises WYE-354. In other embodiments, the at least one additional compound comprises WYE-125132. In other embodiments, the at least one additional compound comprises WYE-687. In other embodiments, the at least one additional compound comprises BGT226. In other embodiments, the at least one additional compound comprises Palomid-529. In other embodiments, the at least one additional compound comprises Gedatolisib. In other embodiments, the at least one additional compound comprises PP121. In other embodiments, the at least one additional compound comprises CH5 132799. In other embodiments, the at least one additional compound comprises Way-600. In other embodiments, the at least one additional compound comprises ETP-46464. In other embodiments, the at least one additional compound comprises GDC-0349. In other embodiments, the at least one additional compound comprises XL388. In other embodiments, the at least one additional compound comprises Zotarolimus. In other embodiments, the at least one additional compound comprises any at least one additional compound described herein, or a salt, solvate, enantiomer or diastereoisomer thereof. In other embodiments, the at least one additional compound is at least one selected from the group consisting of rapamycin, Ridaforolimus, and Everolimus, or a salt, solvate, enantiomer or diastereoisomer thereof. In other embodiments, the at least one additional compound is an active mTOR inhibitor.

The compounds of the present application may possess one or more stereocenters, and each stereocenter may exist independently in either the (R) or(S) configuration. In certain embodiments, compounds described herein are present in optically active or racemic forms. The compounds described herein encompass racemic, optically active, regioisomeric and stereoisomeric forms, or combinations thereof that possess the therapeutically useful properties described herein. Preparation of optically active forms is achieved in any suitable manner, including by way of non-limiting example, by resolution of the racemic form with recrystallization techniques, synthesis from optically active starting materials, chiral synthesis, or chromatographic separation using a chiral stationary phase. A compound illustrated herein by the racemic formula further represents either of the two enantiomers or mixtures thereof, or in the case where two or more chiral center are present, all diastereomers or mixtures thereof.

A synergistic effect may be calculated, for example, using suitable methods such as, for example, the Sigmoid-Emax equation (Holford & Scheiner, 1981, Clin. Pharmacokinet. 6:429-453), the equation of Loewe additivity (Loewe & Muischnek, 1926, Arch. Exp. Pathol Pharmacol. 114:313-326) and the median-effect equation (Chou & Talalay, 1984, Adv. Enzyme Regul. 22:27-55). Each equation referred to above may be applied to experimental data to generate a corresponding graph to aid in assessing the effects of the drug combination. The corresponding graphs associated with the equations referred to above are the concentration-effect curve, isobologram curve and combination index curve, respectively.

### Kits

The present application includes a kit comprising at least one composition of the present application, an applicator, and an instructional material for use thereof.

The instructional material included in the kit comprises instructions for preventing or treating a disease or disorder contemplated within the present application. The instructional material recites the amount of, and frequency with which, at least one composition of the present application should be administered to the mammal. In other embodiments, the kit further comprises at least one additional agent that prevents or treats the disease or disorder contemplated within the present application.

Those skilled in the art will recognize or be able to ascertain using no more than routine experimentation, numerous equivalents to the specific procedures, embodiments, claims, and examples described herein. Such equivalents were considered to be within the scope of this present application and covered by the claims appended hereto. For example, it should be understood, that modifications in reaction conditions, including but not limited to reaction times, reaction size/volume, and experimental reagents, such as solvents, catalysts, pressures, atmospheric conditions, and reducing/oxidizing agents, with art-recognized alternatives and using no more than routine experimentation, are within the scope of the present application.

The following examples further illustrate aspects of the present application. However, they are in no way a limitation of the teachings or disclosure of the present application as set forth herein.

### Experimental Examples

The present application is further described in detail by reference to the following experimental examples. These examples are provided for purposes of illustration only, and are not intended to be limiting unless otherwise specified. Thus, the present application should in no way be construed as being limited to the following examples, but rather, should be construed to encompass any and all variations which become evident as a result of the teaching provided herein.

### Example 1: Intramuscular Coformulation

This is a within-subject, randomized, double-blind, placebo-controlled crossover trial evaluating intramuscular coformulation of an exemplary NMDA receptor antagonist (ketamine) and an exemplary opioid receptor antagonist (naltrexone) in treating major depressive disorder (MDD) and substance use disorder (SUD). It is evaluated whether intramuscular coformulation of ketamine and naltrexone is safe and effective in reducing depression, substance use, and the abuse liability of ketamine. The study is described as follows.

### Research Design

A human laboratory study is conducted to test whether intramuscular (IM) coformulation of ketamine and naltrexone is safe and effective for treating comorbid MDD and SUD (n=40). Particularly, this study evaluates whether placebo (midazolam 0.03 mg/kg+naltrexone 0 mg) and 3 different doses of IM naltrexone (25 mg vs. 50 mg vs. 100 mg)+ketamine (0.5 mg/kg) have different antidepressant effects (Table 3).

TABLE 3
Intramuscular Coformulation Research Design

| | | 4 Test Conditions (randomly assigned on 4 test days) |
|---|---|---|
| 1 | Placebo | IM Midazolam 0.03 mg/kg + IM Naltrexone 0 mg |
| 2 | Low dose | IM Ketamine 0.5 mg/kg + IM Naltrexone 25 mg |
| 3 | Medium | IM Ketamine 0.5 mg/kg + IM Naltrexone 50 mg |
| | dose | |
| 4 | High dose | IM Ketamine 0.5 mg/kg + IM Naltrexone 100 mg |

A within-subject, randomized, double-blind, placebo-controlled crossover trial is proposed, in which each subject completes 1 screening day and 4 test days (Visit 1 for screening, Visit 2 for Test Day #1, Visit 3 for Test Day #2, Visit 3 for Test Day #3, Visit 4 for Test Day #4). The 4 test conditions is randomly assigned on 4 test days. On 4 test days, subjects randomly receive (1) placebo (IM Midazolam 0.03 mg/kg+IM Naltrexone 0 mg), (2) IM Ketamine 0.5 mg/kg+IM Naltrexone 25 mg. (3) IM Ketamine 0.5 mg/kg+IM Naltrexone 50 mg, or (4) IM Ketamine 0.5 mg/kg+IM Naltrexone 100 mg in a crossover design. Each test day are separated at least 10 days apart.

### Study Subjects

A total of 40 subjects with MDD and SUD are enrolled into the study. Subjects are recruited through clinical referrals, flyers, research websites, online advertising, research boards, and posters. Subjects are identified via their response to advertisements and/or internal recruiting through research clinics and other health clinics. Subjects are asked to call at the number provided on the flyers if they are interested in participating in the research study. Patients were recruited in outpatient settings as they met the following criteria.

Inclusion Criteria: (1) Male or female, 21-65 years old; (2) Current major depressive disorder without psychotic features by DSM-5; (3) Montgomery-Åsberg Depression Rating Scale (MADRS)≥20; (4) Current substance use disorder by DSM-5; (5) Abstinence from substance (e.g., alcohol, cocaine, opioid) for >5 days prior to ketamine injection; (6) Able to provide written informed consent.

Exclusion Criteria: (1) Current or past history of psychotic features or psychotic disorder; (2) Current or past history of delirium or dementia; (3) Current uncontrolled hypertension (systolic BP>170 mm Hg or diastolic BP>100 mm Hg); (4) Unstable medical condition or allergy to ketamine or naltrexone, as clinically determined by a physician; (5) Imminent suicidal or homicidal risk; (6) Pregnant or nursing women, positive pregnancy test, or inadequate birth control methods in women of childbearing potential; (7) Positive opioid drug screen test; (8) Opioid use within 10 days prior to study medication (injectable naltrexone); (9) Liver enzymes that are three times higher than the upper limit of normal.

### Procedures

Each subject completes 1 screening day and 4 test days (Visit 1 for screening, Visit 2 for Test Day #1, Visit 3 for Test Day #2, Visit 3 for Test Day #3, Visit 4 for Test Day #4). On 4 test days, subjects randomly receive (1) placebo (IM Midazolam 0.03 mg/kg+IM Naltrexone 0 mg), (2) IM Ketamine 0.5 mg/kg+IM Naltrexone 25 mg, (3) IM Ketamine 0.5 mg/kg+IM Naltrexone 50 mg, or (4) IM Ketamine 0.5 mg/kg+IM Naltrexone 100 mg in a crossover design (Table 4). Each test day is separated at least 10 days apart. All subjects receive usual standard care during this trial.

TABLE 4
Procedures involved from screening visit and 4 test days

| | | | Visit 3 | Visit 4 | Visit 5 |
|---|---|---|---|---|---|
| | | Visit 2 | Test | Test | Test |
| | Visit 1 | Randomized | Day | Day | Day |
| Procedures | Screening | Test Day #1 | #2 | #3 | #4 |
| IM ketamine (or | | X | X | X | X |
| midazolam) + | | | | | |
| IM naltrexone | | | | | |
| Informed | X | | | | |
| Consent | | | | | |
| Inclusion/ | X | | | | |
| Exclusion | | | | | |
| Demographics | X | | | | |
| MINI | X | | | | |
| FHAM | X | | | | |
| Psychiatric | X | | | | |
| Evaluation | | | | | |
| Physical | X | | | | |
| Examination | | | | | |
| Liver Profile, | X | | | | |
| GGT | | | | | |
| CBC, Chemistry | X | | | | |
| 7 | | | | | |
| Urine Tox/Preg | X | X | X | X | X |
| Urinalysis | X | | | | |
| BP, Pulse, | X | X | X | X | X |
| Weight | | | | | |
| MADRS, QIDS- | X | X | X | X | X |
| SR, HAM-A | | | | | |
| C-SSRS | X | X | X | X | X |
| VAS (craving) | X | X | X | X | X |
| BPRS, CADSS, | | X | X | X | X |
| VAS (mood) | | | | | |
| Concomitant | X | X | X | X | X |
| Meds | | | | | |
| TLFB | X | X | X | X | X |
| CIWA-Ar | X | X | X | X | X |
| Breathalyzer | X | X | X | X | X |
| Adverse Event | | X | X | X | X |
| DAPS | | X | X | X | X |

### Abbreviations:

Mini International Neuropsychiatric Interview (MINI); Family History Assessment Module (FHAM), Columbia-Suicide Severity Rating Scale (C-SSRS), Montgomery-Åsberg Depression Rating Scale (MADRS), Quick Inventory of Depressive Symptomatology-Self Report (QIDS-SR), Hamilton Anxiety Rating Scale (HAM-A), Time Line Follow Back (TLFB), Brief Psychiatric Rating Scale (BPRS), Clinician-Administered Dissociative States Scale (CADSS), Visual Analog Scale (VAS) of Mood States, Complete Blood Count (CBC), Visual Analog Scale (VAS) of Craving, Clinical Institute Withdrawal Assessment for Alcohol, Revised (CIWA-Ar), Drug Abuse Potential Scale (DAPS)

### Initial Telephone Screening

Subject eligibility are first assessed via telephone screening and a preliminary medical record review. Telephone screening occurs by experienced research personnel adept with this process. If the subject seems to be a likely candidate for inclusion in this protocol, he or she is invited to a screening visit for study eligibility.

### Screening Visit

At the screening visit, the research team explains the overall study plan to potential subjects and go over the consent form. Subjects have an initial screening evaluation that includes psychiatric history, medical history, mental status examination, physical examination, and laboratory assessment. Other detailed procedures are described in Table 4. If the examination and test results are acceptable and the subject meets the inclusion/exclusion criteria, the subject is invited to the baseline visit.

### Four Test Days (Receiving IM Ketamine (or Midazolam)+IM Naltrexone, Table 5)

TABLE 5
Four Test Days (receiving IM ketamine (or midazolam) + IM naltrexone)

| Time | Procedures |
|---|---|
| T − 60 min | Vital signs (blood pressure, pulse) |
| | Urine Toxicology/Pregnancy |
| | VAS (craving), MADRS, QIDS-SR, |
| | VAS (mood), HAM-A, BPRS, CADSS* |
| T0 | IM ketamine (or midazolam) + IM naltrexone |
| | administered |
| T + 60 min | Vital signs, VAS (craving), MADRS, QIDS-SR, |
| | VAS (mood), HAM-A, BPRS, CADSS, DAPS |
| T + 240 min | Vital signs, VAS (craving), MADRS, QIDS-SR, |
| | VAS (mood), HAM-A, BPRS, CADSS |

If urine toxicology is positive for alcohol or illicit drugs (except marijuana), IM ketamine (or midazolam)+IM naltrexone administration session is cancelled and rescheduled. *Items that could not change within a session (e.g., sleep, appetite) will be rated only at T-60 and at follow-up sessions. After the subject receives study medication, he or she receives lunch and is medically cleared before leaving. If necessary, intravenous lorazepam (ativan) at 1 mg is available to reduce the behavioral effects of ketamine. This is determined by a research physician. Subjects are not permitted to drive to and from the hospital on medication treatment days, and subjects must make their own arrangements for a ride. If this is not possible, the study organizers provide transportation for the subject.

### Measures

Medical Assessments: Physical examination (including vital sign determination) and clinical laboratories are completed at the first visit. Urine toxicology screen and breathalyzer are performed on the morning of each infusion day, and the results are determined before proceeding with the infusions. The subject does not receive study medications on the day if urine toxicology results are positive for any illicit drugs (except marijuana) or if his/her breathalyzer is positive for alcohol. A pregnancy test is also administered to all reproductive age females enrolled in the study prior to participation.

Psychiatric Assessments: Ratings were performed by trained research assistants. Administration of clinical measures was supervised by the primary investigator. Each of the psychiatric assessment instruments is briefly described below. (1) Mini International Neuropsychiatric Interview (MINI): The Mini International Neuropsychiatric Interview (MINI) will be used to classify subjects according to the presence or absence of psychiatric disorders based on the DSM-5 criteria. (2) Montgomery-Asberg Depression Rating Scale (MADRS): The MADRS is a standardized instrument to ascertain depressed mood and neurovegetative signs and symptoms of a major depressive episode. (3) Quick Inventory of Depressive Symptomatology-Self Report (QIDS-SR): The QIDS-SR is a patient-rated depression instrument. (4) Hamilton Anxiety Rating Scale (HAM-A): The HAM-A is a standardized instrument to evaluate anxiety severity. (5) Brief Psychotic Rating Scale (BPRS): The BPRS is a standardized instrument that contains scales assessing psychotic symptoms including positive and negative symptoms, activation and emotional distress. (6) Clinician-Administered Dissociative States Scale (CADSS): The CADSS has self and interviewer-administered items including 5 subscales, generated a priori, evaluating dissociation including altered environmental perception, time perception, spatial/body perception, derealization and memory impairment. (7) Visual Analog Scale (VAS) of Mood States: The VAS includes scales for anxiety, drowsiness, high irritability, anger and sadness. The scales are 100 mm lines marked by subjects at a point corresponding to the apparent intensity of the feeling state (0=none, to 100=most ever). (8) Visual Analog Scale (VAS) of craving: The scales are 100 mm lines marked by subjects at a point corresponding to the apparent intensity of craving for their primary drug (0=none, to 100=most ever). (9) Time Line Follow Back (TLFB): The TLFB is a standardized measure utilized for collecting information on daily alcohol use as well as other substance use. (10) Columbia-Suicide Severity Rating Scale (C-SSRS): The C-SSRS has both lifetime/recent and since last visit versions. The “Lifetime/Recent” version gathers information on lifetime history of suicidality and recent suicidal ideation/self-injurious behavior. The “Since Last Visit” version of the C-SSRS asks about any suicidal thoughts or behaviors the subject has exhibited since the last time administered the C-SSRS. (11) Clinical Institute Withdrawal Assessment for Alcohol, Revised (CIWA-Ar): The CIWA-Ar is a reliable and valid 10-item assessment for monitoring alcohol withdrawal symptoms. (12) Drug Abuse Potential Scale (DAPS): The DAPS will be used to assess abuse liability of study drugs.

### Outcome Measures

Primary outcome measure: (1) MDD: Clinical response in symptoms of MDD as measured by the Montgomery-Asberg Depression Rating Scale (MADRS) (response defined as a ≥50% improvement from baseline in MADRS score). (2) SUD: Substance use as measured by the Time Line Follow Back (TLFB).

Exploratory outcome measures: (3) Remission in symptoms of MDD as measured by the MADRS (remission defined as a MADRS score≤9). (4) Primary substance craving as measured by the VAS (craving). (5) Abuse potential of ketamine as measured by the Drug Abuse Potential Scale.

### Example 2: Intranasal Coformulation

This is a within-subject, randomized, double-blind, placebo-controlled crossover trial evaluating intranasal coformulation of an exemplary NMDA receptor antagonist (ketamine) and an exemplary opioid receptor antagonist (naltrexone) in treating major depressive disorder (MDD) and substance use disorder (SUD). It is evaluated whether intranasal coformulation of ketamine and naltrexone is safe and effective in reducing depression, substance use, and the abuse liability of ketamine. The study is described as follows.

### Research Design

A human laboratory study is conducted to test whether intranasal (IN) coformulation of ketamine and naltrexone is safe and effective for treating comorbid MDD and SUD (n=40). Particularly, this study evaluates whether placebo (saline solution) and 3 different doses of IN naltrexone (2 mg vs. 4 mg vs. 10 mg)+ketamine (50 mg) would have different antidepressant effects (Table 6).

TABLE 6
Intranasal Coformulation Research Design

| | | 4 Test Conditions (randomly assigned on 4 test days) |
|---|---|---|
| 1 | Placebo | IN Saline solution |
| 2 | Low dose | IN Ketamine 50 mg + IN Naltrexone 2 mg |
| 3 | Medium | IN Ketamine 50 mg + IN Naltrexone 4 mg |
| | dose | |
| 4 | High dose | IN Ketamine 50 mg + IN Naltrexone 10 mg |

A within-subject, randomized, double-blind, placebo-controlled crossover trial is proposed, in which each subject would complete 1 screening day and 4 test days (Visit 1 for screening, Visit 2 for Test Day #1, Visit 3 for Test Day #2, Visit 3 for Test Day #3, Visit 4 for Test Day #4). The 4 test conditions are randomly assigned on 4 test days. On 4 test days, subjects randomly receive (1) placebo (IN Saline solution), (2) IN Ketamine 50 mg+IN Naltrexone 2 mg, (3) IN Ketamine 50 mg+IN Naltrexone 4 mg, or (4) IN Ketamine 50 mg+IN Naltrexone 10 mg in a crossover design. Each test day is separated at least 10 days apart.

### Study Subjects

A total of 40 subjects with MDD and SUD are enrolled into the study. Patients were recruited in outpatient settings as they met the following criteria.

Inclusion Criteria: (1) Male or female, 21-65 years old. (2) Current major depressive disorder without psychotic features by DSM-5. (3) Montgomery-Åsberg Depression Rating Scale (MADRS)≥20. (4) Current substance use disorder by DSM-5. (5) Abstinence from substance (e.g., alcohol, cocaine, opioid) for >5 days prior to ketamine injection. (6) Able to provide written informed consent.

Exclusion Criteria: (1) Current or past history of psychotic features or psychotic disorder. (2) Current or past history of delirium or dementia. (3) Current uncontrolled hypertension (systolic BP>170 mm Hg or diastolic BP>100 mm Hg). (4) Unstable medical condition or allergy to ketamine or naltrexone, as clinically determined by a physician. (5) Imminent suicidal or homicidal risk. (6) Pregnant or nursing women, positive pregnancy test, or inadequate birth control methods in women of childbearing potential. (7) Positive opioid drug screen test. (8) Opioid use within 10 days prior to study medication (injectable naltrexone). (9) Liver enzymes that are three times higher than the upper limit of normal

### Procedures

Each subject completes 1 screening day and 4 test days (Visit 1 for screening, Visit 2 for Test Day #1, Visit 3 for Test Day #2, Visit 3 for Test Day #3, Visit 4 for Test Day #4). On 4 test days, subjects randomly receive (1) placebo (IN Saline solution), (2) IN Ketamine 50 mg+IN Naltrexone 2 mg, (3) IN Ketamine 50 mg+IN Naltrexone 4 mg, or (4) IN Ketamine 50 mg+IN Naltrexone 10 mg in a crossover design (Table 7). Each test day is separated at least 10 days apart. All patients receive usual standard care during this trial.

TABLE 7
Procedures involved from screening visit and 3 test days

| | | Visit 2 | Visit 3 | Visit 4 | Visit 5 |
|---|---|---|---|---|---|
| | | Ran- | Test | Test | Test |
| | Visit 1 | domized | Day | Day | Day |
| Procedures | Screening | Test Day #1 | #2 | #3 | #4 |
| IN ketamine (or | | X | X | X | X |
| placebo) + IN | | | | | |
| naltrexone | | | | | |
| (or placebo) | | | | | |
| Informed Consent | X | | | | |
| Inclusion/Exclusion | X | | | | |
| Demographics | X | | | | |
| MINI | X | | | | |
| FHAM | X | | | | |
| Psychiatric | X | | | | |
| Evaluation | | | | | |
| Physical | X | | | | |
| Examination | | | | | |
| Liver Profile, GGT | X | | | | |
| CBC, Chemistry 7 | X | | | | |
| Urine Tox/Preg | X | X | X | X | X |
| Urinalysis | X | | | | |
| BP, Pulse, Weight | X | X | X | X | X |
| MADRS, QIDS-SR, | X | X | X | X | X |
| HAM-A | | | | | |
| C-SSRS | X | X | X | X | X |
| VAS (craving) | X | X | X | X | X |
| BPRS, CADSS, | | X | X | X | X |
| VAS (mood) | | | | | |
| Concomitant Meds | X | X | X | X | X |
| TLFB | X | X | X | X | X |
| CIWA-Ar | X | X | X | X | X |
| Breathalyzer | X | X | X | X | X |
| Adverse Event | | X | X | X | X |
| DAPS | | X | X | X | X |

### Abbreviations:

Mini International Neuropsychiatric Interview (MINI); Family History Assessment Module (FHAM), Columbia-Suicide Severity Rating Scale (C-SSRS), Montgomery-Åsberg Depression Rating Scale (MADRS), Quick Inventory of Depressive Symptomatology-Self Report (QIDS-SR), Hamilton Anxiety Rating Scale (HAM-A), Time Line Follow Back (TLFB), Brief Psychiatric Rating Scale (BPRS), Clinician-Administered Dissociative States Scale (CADSS), Visual Analog Scale (VAS) of Mood States, Complete Blood Count (CBC), Visual Analog Scale (VAS) of Craving, Clinical Institute Withdrawal Assessment for Alcohol, Revised (CIWA-Ar), Drug Abuse Potential Scale (DAPS)

### Initial Telephone Screening

Subject eligibility is first assessed via telephone screening and a preliminary medical record review. Telephone screening occurs by experienced research personnel adept with this process. If the subject seems to be a likely candidate for inclusion in this protocol, he or she is invited to a screening visit for study eligibility.

### Screening Visit

At the screening visit, the research team explains the overall study plan to potential subjects and go over the consent form. Subjects have an initial screening evaluation that includes psychiatric history, medical history, mental status examination, physical examination, and laboratory assessment. Other detailed procedures are described in Table 7. If the examination and test results are acceptable and the subject meets the inclusion/exclusion criteria, the subject is invited to the baseline visit.

### Four Test Days (Receiving IN Ketamine (or Placebo)+IN Naltrexone (or Placebo), Table 8)

TABLE 8
Four Test Days (IN ketamine (or placebo) + IN naltrexone (or placebo)

| Time | Procedures |
|---|---|
| T − 60 min | Vital signs (blood pressure, pulse) |
| | Urine Toxicology/Pregnancy |
| | VAS (craving), MADRS, QIDS-SR, VAS |
| | (mood), HAM-A, BPRS, CADSS* |
| T0 | IN ketamine (or placebo) + IN naltrexone |
| | (or placebo) administered |
| T + 60 min | Vital signs, VAS (craving), MADRS, |
| | QIDS-SR, VAS (mood), HAM-A, |
| | BPRS, CADSS, DAPS |
| T + 240 min | Vital signs, VAS (craving), MADRS, |
| | QIDS-SR, VAS (mood), HAM-A, |
| | BPRS, CADSS |

If urine toxicology is positive for alcohol or illicit drugs (except marijuana), IN ketamine (or placebo)+IN naltrexone (or placebo) administration session is cancelled and rescheduled. *Items that could not change within a session (e.g., sleep, appetite) will be rated only at T-60 and at follow-up sessions. After the subject receives study medication, he or she receives lunch and is medically cleared before leaving. If necessary, intravenous lorazepam (ativan) at 1 mg is available to reduce the behavioral effects of ketamine. This is determined by a research physician. Subjects are not permitted to drive to and from the hospital on medication treatment days, and subjects must make their own arrangements for a ride. If this is not possible, the study organizers provide transportation for the subject.

### Outcome Measures

Primary outcome measure: (1) MDD: Clinical response in symptoms of MDD as measured by the Montgomery-Asberg Depression Rating Scale (MADRS) (response defined as a ≥50% improvement from baseline in MADRS score). (2) SUD: Substance use as measured by the Time Line Follow Back (TLFB).

Exploratory outcome measures: (3) Remission in symptoms of MDD as measured by the MADRS (remission defined as a MADRS score≤9). (4) Primary substance craving as measured by the VAS (craving). (5) Abuse potential of ketamine as measured by the Drug Abuse Potential Scale.

### Example 3: NMDA Receptor Negative Allosteric Modulator/Partial Agonist and μ-Opioid Receptor Negative Allosteric Modulator/Partial Agonist

This is a within-subject, randomized, double-blind, placebo-controlled crossover trial evaluating intramuscular coformulation of an exemplary NMDA receptor negative allosteric modulator/partial agonist and an exemplary μ-opioid receptor negative allosteric modulator/partial agonist in treating major depressive disorder (MDD) and substance use disorder (SUD). It is evaluated whether intramuscular coformulation of the exemplary NMDA receptor negative allosteric modulator/partial agonist and the exemplary μ-opioid receptor negative allosteric modulator/partial agonist is safe and effective in reducing depression, substance use, and the abuse liability of the exemplary NMDA receptor negative allosteric modulator/partial agonist. The study is described as follows.

### Research Design

A human laboratory study is conducted to test whether intramuscular (IM) coformulation of the exemplary NMDA receptor negative allosteric modulator/partial agonist and the exemplary μ-opioid receptor negative allosteric modulator/partial agonist is safe and effective for treating comorbid MDD and SUD (n=40). Particularly, this study evaluates whether placebo (midazolam+the exemplary μ-opioid receptor negative allosteric modulator/partial agonist 0 mg) and 3 different doses of IM the exemplary μ-opioid receptor negative allosteric modulator/partial agonist (low dose vs. medium dose vs. high dose)+the exemplary NMDA receptor negative allosteric modulator/partial agonist would have different antidepressant effects (Table 9).

TABLE 9
Intramuscular Coformulation Research Design

| | | 4 Test Conditions (randomly assigned on 4 test days) |
|---|---|---|
| 1 | Placebo | IM Midazolam + IM the exemplary |
| | | μ-opioid receptor negative |
| | | allosteric modulator/partial agonist 0 mg |
| 2 | Low | IM the exemplary NMDA receptor negative allosteric |
| | dose | modulator/partial agonist + IM the |
| | | exemplary μ-opioid receptor |
| | | negative allosteric modulator/partial agonist low dose |
| 3 | Medium | IM the exemplary NMDA receptor negative allosteric |
| | dose | modulator/partial agonist + IM the |
| | | exemplary μ-opioid receptor |
| | | negative allosteric modulator/partial agonist medium dose |
| 4 | High | IM the exemplary NMDA receptor negative allosteric |
| | dose | modulator/partial agonist + IM the |
| | | exemplary μ-opioid receptor |
| | | negative allosteric modulator/partial agonist high dose |

A within-subject, randomized, double-blind, placebo-controlled crossover trial is proposed, in which each subject would complete 1 screening day and 4 test days (Visit 1 for screening, Visit 2 for Test Day #1, Visit 3 for Test Day #2, Visit 3 for Test Day #3, Visit 4 for Test Day #4). The 4 test conditions are randomly assigned on 4 test days. On 4 test days, subjects randomly receive (1) placebo (IM Midazolam+IM the exemplary μ-opioid receptor negative allosteric modulator/partial agonist 0 mg), (2) IM the exemplary NMDA receptor negative allosteric modulator/partial agonist+IM the exemplary μ-opioid receptor negative allosteric modulator/partial agonist low dose, (3) IM the exemplary NMDA receptor negative allosteric modulator/partial agonist+IM The exemplary μ-opioid receptor negative allosteric modulator/partial agonist medium dose, or (4) IM the exemplary NMDA receptor negative allosteric modulator/partial agonist+IM the exemplary μ-opioid receptor negative allosteric modulator/partial agonist high dose in a crossover design. Each test day is separated at least 10 days apart.

### Study Subjects

A total of 40 subjects with MDD and SUD is enrolled into the study. Subjects are recruited through clinical referrals, flyers, research websites, online advertising, research boards, and posters. Subjects are identified via their response to advertisements and/or internal recruiting through research clinics and other health clinics. Subjects are asked to call at the number provided on the flyers if they are interested in participating in the research study. Patients were recruited in outpatient settings as they met the following criteria.

Inclusion Criteria: (1) Male or female, 21-65 years old; (2) Current major depressive disorder without psychotic features by DSM-5; (3) Montgomery-Asberg Depression Rating Scale (MADRS)≥20; (4) Current substance use disorder by DSM-5; (5) Abstinence from substance (e.g., alcohol, cocaine, opioid) for >5 days prior to the exemplary NMDA receptor negative allosteric modulator injection; (6) Able to provide written informed consent.

Exclusion Criteria: (1) Current or past history of psychotic features or psychotic disorder; (2) Current or past history of delirium or dementia; (3) Current uncontrolled hypertension (systolic BP>170 mm Hg or diastolic BP>100 mm Hg); (4) Unstable medical condition or allergy to the exemplary NMDA receptor negative allosteric modulator or the exemplary μ-opioid receptor negative allosteric modulator/partial agonist, as clinically determined by a physician; (5) Imminent suicidal or homicidal risk; (6) Pregnant or nursing women, positive pregnancy test, or inadequate birth control methods in women of childbearing potential; (7) Positive opioid drug screen test; (8) Opioid use within 10 days prior to study medication; (9) Liver enzymes that are three times higher than the upper limit of normal.

### Procedures

Each subject would complete 1 screening day and 4 test days (Visit 1 for screening, Visit 2 for Test Day #1, Visit 3 for Test Day #2, Visit 3 for Test Day #3, Visit 4 for Test Day #4). On 4 test days, subjects randomly receive (1) placebo (IM Midazolam+IM the exemplary μ-opioid receptor negative allosteric modulator/partial agonist 0 mg), (2) IM the exemplary NMDA receptor negative allosteric modulator/partial agonist+IM the exemplary μ-opioid receptor negative allosteric modulator/partial agonist low dose, (3) IM the exemplary NMDA receptor negative allosteric modulator/partial agonist+IM the exemplary μ-opioid receptor negative allosteric modulator/partial agonist medium dose, or (4) IM the exemplary NMDA receptor negative allosteric modulator/partial agonist+IM The exemplary μ-opioid receptor negative allosteric modulator/partial agonist high dose in a crossover design (Table 10). Each test day is separated at least 10 days apart. All subjects receive usual standard care during this trial.

TABLE 10
Procedures involved from screening visit and 4 test days

| | | Visit 2 | Visit 3 | Visit 4 | Visit 5 |
|---|---|---|---|---|---|
| | | Randomized | Test | Test | Test |
| | Visit 1 | Test Day | Day | Day | Day |
| Procedures | Screening | #1 | #2 | #3 | #4 |
| IM the exemplary | | X | X | X | X |
| NMDA receptor | | | | | |
| negative allosteric | | | | | |
| modulator/partial | | | | | |
| agonist | | | | | |
| (or midazolam) | | | | | |
| + | | | | | |
| IM the exemplary μ- | | | | | |
| opioid receptor | | | | | |
| negative allosteric | | | | | |
| modulator/partial | | | | | |
| agonist | | | | | |
| Informed Consent | X | | | | |
| Inclusion/Exclusion | X | | | | |
| Demographics | X | | | | |
| MINI | X | | | | |
| FHAM | X | | | | |
| Psychiatric | X | | | | |
| Evaluation | | | | | |
| Physical | X | | | | |
| Examination | | | | | |
| Liver Profile, GGT | X | | | | |
| CBC, Chemistry 7 | X | | | | |
| Urine Tox/Preg | X | X | X | X | X |
| Urinalysis | X | | | | |
| BP, Pulse, Weight | X | X | X | X | X |
| MADRS, QIDS-SR, | X | X | X | X | X |
| HAM-A | | | | | |
| C-SSRS | X | X | X | X | X |
| VAS (craving) | X | X | X | X | X |
| BPRS, CADSS, | | X | X | X | X |
| VAS (mood) | | | | | |
| Concomitant Meds | X | X | X | X | X |
| TLFB | X | X | X | X | X |
| CIWA-Ar | X | X | X | X | X |
| Breathalyzer | X | X | X | X | X |
| Adverse Event | | X | X | X | X |
| DAPS | | X | X | X | X |

### Abbreviations:

Mini International Neuropsychiatric Interview (MINI); Family History Assessment Module (FHAM), Columbia-Suicide Severity Rating Scale (C-SSRS), Montgomery-Åsberg Depression Rating Scale (MADRS), Quick Inventory of Depressive Symptomatology-Self Report (QIDS-SR), Hamilton Anxiety Rating Scale (HAM-A), Time Line Follow Back (TLFB), Brief Psychiatric Rating Scale (BPRS), Clinician-Administered Dissociative States Scale (CADSS), Visual Analog Scale (VAS) of Mood States, Complete Blood Count (CBC), Visual Analog Scale (VAS) of Craving, Clinical Institute Withdrawal Assessment for Alcohol, Revised (CIWA-Ar), Drug Abuse Potential Scale (DAPS)

### Initial Telephone Screening

Subject eligibility is first assessed via telephone screening and a preliminary medical record review. Telephone screening occurs by experienced research personnel adept with this process. If the subject seems to be a likely candidate for inclusion in this protocol, he or she is invited to a screening visit for study eligibility.

### Screening Visit

At the screening visit, the research team explains the overall study plan to potential subjects and go over the consent form. Subjects have an initial screening evaluation that includes psychiatric history, medical history, mental status examination, physical examination, and laboratory assessment. If the examination and test results are acceptable and the subject meets the inclusion/exclusion criteria, the subject is invited to the baseline visit.

Four Test Days (receiving IM the exemplary NMDA receptor negative allosteric modulator/partial agonist (or midazolam)+IM the exemplary μ-opioid receptor negative allosteric modulator/partial agonist, Table 11)

TABLE 11
Four Test Days (receiving IM the exemplary NMDA receptor negative
allosteric modulator (or midazolam) + IM the exemplary μ-opioid
receptor negative allosteric modulator/partial agonist)

| Time | Procedures |
|---|---|
| T − 60 min | Vital signs (blood pressure, pulse) |
| | Urine Toxicology/Pregnancy |
| | VAS (craving), MADRS, QIDS-SR, VAS |
| | (mood), HAM-A, BPRS, CADSS* |
| T0 | IM the exemplary NMDA receptor |
| | negative allosteric modulator (or |
| | midazolam) + IM the exemplary μ-opioid |
| | receptor negative allosteric |
| | modulator/partial agonist administered |
| T + 60 min | Vital signs, VAS (craving), MADRS, |
| | QIDS-SR, VAS (mood), HAM-A, |
| | BPRS, CADSS, DAPS |
| T + 240 min | Vital signs, VAS (craving), MADRS, |
| | QIDS-SR, VAS (mood), HAM-A, |
| | BPRS, CADSS |

If urine toxicology is positive for alcohol or illicit drugs (except marijuana), IM the exemplary NMDA receptor negative allosteric modulator/partial agonist (or midazolam)+IM the exemplary μ-opioid receptor negative allosteric modulator/partial agonist administration session is cancelled and rescheduled. *Items that could not change within a session (e.g., sleep, appetite) are rated only at T-60 and at follow-up sessions. After the subject receives study medication, he or she receives lunch and is medically cleared before leaving. Subjects are not permitted to drive to and from the hospital on medication treatment days, and subjects must make their own arrangements for a ride. If this is not possible, the study organizer provides transportation for the subject.

### Measures

Medical Assessments: Physical examination (including vital sign determination) and clinical laboratories are completed at the first visit. Urine toxicology screen and breathalyzer are performed on the morning of each infusion day, and the results re determined before proceeding with the infusions. The subject does not receive study medications on the day if urine toxicology results are positive for any illicit drugs (except marijuana) or if his/her breathalyzer is positive for alcohol. A pregnancy test is also administered to all reproductive age females enrolled in the study prior to participation.

Psychiatric Assessments: Ratings were performed by trained research assistants. Administration of clinical measures was supervised by the primary investigator. Each of the psychiatric assessment instruments is briefly described below. (1) Mini International Neuropsychiatric Interview (MINI): The Mini International Neuropsychiatric Interview (MINI) will be used to classify subjects according to the presence or absence of psychiatric disorders based on the DSM-5 criteria. (2) Montgomery-Asberg Depression Rating Scale (MADRS): The MADRS is a standardized instrument to ascertain depressed mood and neurovegetative signs and symptoms of a major depressive episode. (3) Quick Inventory of Depressive Symptomatology-Self Report (QIDS-SR): The QIDS-SR is a patient-rated depression instrument. (4) Hamilton Anxiety Rating Scale (HAM-A): The HAM-A is a standardized instrument to evaluate anxiety severity. (5) Brief Psychotic Rating Scale (BPRS): The BPRS is a standardized instrument that contains scales assessing psychotic symptoms including positive and negative symptoms, activation and emotional distress. (6) Clinician-Administered Dissociative States Scale (CADSS): The CADSS has self and interviewer-administered items including 5 subscales, generated a priori, evaluating dissociation including altered environmental perception, time perception, spatial/body perception, derealization and memory impairment. (7) Visual Analog Scale (VAS) of Mood States: The VAS includes scales for anxiety, drowsiness, high irritability, anger and sadness. The scales are 100 mm lines marked by subjects at a point corresponding to the apparent intensity of the feeling state (0)=none, to 100=most ever). (8) Visual Analog Scale (VAS) of craving: The scales are 100 mm lines marked by subjects at a point corresponding to the apparent intensity of craving for their primary drug (0)=none, to 100)=most ever). (9) Time Line Follow Back (TLFB): The TLFB is a standardized measure utilized for collecting information on daily alcohol use as well as other substance use. (10)) Columbia-Suicide Severity Rating Scale (C-SSRS): The C-SSRS has both lifetime/recent and since last visit versions. The “Lifetime/Recent” version gathers information on lifetime history of suicidality and recent suicidal ideation/self-injurious behavior. The “Since Last Visit” version of the C-SSRS asks about any suicidal thoughts or behaviors the subject has exhibited since the last time administered the C-SSRS. (11) Clinical Institute Withdrawal Assessment for Alcohol, Revised (CIWA-Ar): The CIWA-Ar is a reliable and valid 10-item assessment for monitoring alcohol withdrawal symptoms. (12) Drug Abuse Potential Scale (DAPS): The DAPS is used to assess abuse liability of study drugs.

### Outcome Measures

Primary outcome measure: (1) MDD: Clinical response in symptoms of MDD as measured by the Montgomery-Asberg Depression Rating Scale (MADRS) (response defined as a ≥50% improvement from baseline in MADRS score). (2) SUD: Substance use as measured by the Time Line Follow Back (TLFB).

Exploratory outcome measures: (3) Remission in symptoms of MDD as measured by the MADRS (remission defined as a MADRS score≤9). (4) Primary substance craving as measured by the VAS (craving). (5) Abuse potential of the exemplary NMDA receptor negative allosteric modulator as measured by the Drug Abuse Potential Scale.

### Example 4: Measurement of μ-Opioid Receptor Occupancy In Vivo

The following method is one of the ways to measure μ-opioid receptor occupancy while using coformulation of NMDA receptor modulator and opioid receptor modulator. This is a within-subject, randomized crossover trial evaluating intramuscular coformulation of an exemplary NMDA receptor antagonist (ketamine) and an exemplary opioid receptor antagonist (naltrexone) to measure brain μ-opioid receptor occupancy by using positron emission tomography (PET) with the ligand [¹¹C]-carfentanil.

### Research Design

A human laboratory study is conducted to measure μ-opioid receptor occupancy after receiving intramuscular (IM) naltrexone alone vs. IM coformulation of ketamine and naltrexone in patient with comorbid MDD and SUD (n=10) by using PET scans with the ligand [¹¹C]-carfentanil, a highly μ-opioid receptor-selective agonist tracer. As described in Table 12, subjects receive 4 PET scans as follows: (1) PET 1 at baseline and (2) PET 2, 3, and 4 (2 hours after receiving study medications). The 3 test conditions are randomly assigned on 3 test days: (1) naltrexone only (IM saline+IM naltrexone 50 mg), (2) ketamine+naltrexone (low dose) (IM ketamine 0.5 mg/kg+IM naltrexone 25 mg), and (3) ketamine+naltrexone (medium dose) (IM ketamine 0.5 mg/kg+IM naltrexone 50 mg).

One can calculate μ-opioid receptor occupancy (%) as follows:

$$μ - opioid receptor occupancy by opioid receptor modulator ( \% ) = 100 \times ( 1 - \frac{binding potential after medication}{binding potential at baseline} )$$

TABLE 12
Four PET Scans for Measuring Brain μ-Opioid Receptor Occupancy

| | | 3 Test Conditions (randomly |
|---|---|---|
| 4 PET scans | | assigned on 3 test days) |
| PET 1 | Baseline | Before receiving study |
| (Baseline) | | medications |
| PET 2 | Naltrexone only | IM Saline + IM Naltrexone 50 mg |
| (Test Day #1) | | |
| PET 3 | Ketamine + Naltrexone | IM Ketamine 0.5 mg/kg + IM |
| (Test Day #2) | (low dose) | Naltrexone 25 mg |
| PET 4 | Ketamine + Naltrexone | IM Ketamine 0.5 mg/kg + IM |
| (Test Day #3) | (medium dose) | Naltrexone 50 mg |

PET: positron emission tomography

A within-subject, randomized crossover trial is proposed, in which each subject completes 1 screening day, 1 baseline PET scan, and 3 test days (Visit 1 for screening, Visit 2 for baseline PET scan, Visit 3 for Test Day #1, Visit 4 for Test Day #2, Visit 5 for Test Day #3). The 3 test conditions are randomly assigned on 3 test days. On 3 test days, subjects randomly receive (1) naltrexone only (IM saline+IM naltrexone 50 mg), (2) ketamine+naltrexone (low dose) (IM ketamine 0.5 mg/kg+IM naltrexone 25 mg), or (3) ketamine+naltrexone (medium dose) (IM ketamine 0.5 mg/kg+IM naltrexone 50 mg) in a crossover design. Each test day is separated at least 7 days apart.

### Study Subjects

A total of 10 subjects with MDD and SUD are enrolled into the study. Subjects are recruited through clinical referrals, flyers, research websites, online advertising, research boards, and posters. Subjects are identified via their response to advertisements and/or internal recruiting through research clinics and other health clinics. Subjects are asked to call at the number provided on the flyers if they are interested in participating in the research study. Patients were recruited in outpatient settings as they met the following criteria.

Inclusion Criteria: (1) Male or female, 21-65 years old; (2) Current major depressive disorder without psychotic features by DSM-5; (3) Montgomery-Åsberg Depression Rating Scale (MADRS)≥20; (4) Current substance use disorder by DSM-5; (5) Abstinence from substance (e.g., alcohol, cocaine, opioid) for >5 days prior to ketamine injection; (6) Able to provide written informed consent.

Exclusion Criteria: (1) Current or past history of psychotic features or psychotic disorder; (2) Current or past history of delirium or dementia; (3) Current uncontrolled hypertension (systolic BP>170 mm Hg or diastolic BP>100 mm Hg); (4) Unstable medical condition or allergy to ketamine or naltrexone, as clinically determined by a physician; (5) Imminent suicidal or homicidal risk; (6) Pregnant or nursing women, positive pregnancy test, or inadequate birth control methods in women of childbearing potential; (7) Positive opioid drug screen test; (8) Opioid use within 10 days prior to study medication (injectable naltrexone); (9) Liver enzymes that are three times higher than the upper limit of normal.

### Procedures

Each subject completes 1 screening day, 1 baseline PET scan, and 3 test days (Visit 1 for screening, Visit 2 for baseline PET scan, Visit 3 for Test Day #1, Visit 4 for Test Day #2, Visit 5 for Test Day #3). The 3 test conditions are randomly assigned on 3 test days. On 3 test days, subjects andomly receive (1) naltrexone only (IM saline+IM naltrexone 50 mg), (2) ketamine+naltrexone (low dose) (IM ketamine 0.5 mg/kg+IM naltrexone 25 mg), or (3) ketamine+naltrexone (medium dose) (IM ketamine 0.5 mg/kg+IM naltrexone 50 mg) in a crossover design. Each test day will be separated at least 7 days apart.

TABLE 13
Procedures involved from screening visit, baseline PET, and 3 test days

| | | | Visit 3 | Visit 4 | Visit 5 |
|---|---|---|---|---|---|
| | Visit 1 | Visit 2 | Test | Test | Test |
| Procedures | Screening | Randomized | Day #1 | Day #2 | Day #3 |
| PET scan | | PET #1 | PET #2 | PET #3 | PET #4 |
| IM ketamine | | | X | X | X |
| (or saline) + | | | | | |
| IM naltrexone | | | | | |
| Informed Consent | X | | | | |
| Inclusion/Exclusion | X | | | | |
| Demographics | X | | | | |
| MINI | X | | | | |
| FHAM | X | | | | |
| Psychiatric | X | | | | |
| Evaluation | | | | | |
| Physical | X | | | | |
| Examination | | | | | |
| Liver Profile, GGT | X | | | | |
| CBC, Chemistry 7 | X | | | | |
| Urine Tox/Preg | X | X | X | X | X |
| Urinalysis | X | | | | |
| BP, Pulse, Weight | X | X | X | X | X |
| MADRS, QIDS-SR, | X | X | X | X | X |
| HAM-A | | | | | |
| C-SSRS | X | X | X | X | X |
| VAS (craving) | X | X | X | X | X |
| BPRS, CADSS, | | X | X | X | X |
| VAS (mood) | | | | | |
| Concomitant Meds | X | X | X | X | X |
| TLFB | X | X | X | X | X |
| CIWA-Ar | X | X | X | X | X |
| Breathalyzer | X | X | X | X | X |
| Adverse Event | | X | X | X | X |
| DAPS | | X | X | X | X |

### Abbreviations:

Mini International Neuropsychiatric Interview (MINI): Family History Assessment Module (FHAM), Columbia-Suicide Severity Rating Scale (C-SSRS), Montgomery-Åsberg Depression Rating Scale (MADRS), Quick Inventory of Depressive Symptomatology-Self Report (QIDS-SR), Hamilton Anxiety Rating Scale (HAM-A), Time Line Follow Back (TLFB), Brief Psychiatric Rating Scale (BPRS), Clinician-Administered Dissociative States Scale (CADSS), Visual Analog Scale (VAS) of Mood States, Complete Blood Count (CBC), Visual Analog Scale (VAS) of Craving, Clinical Institute Withdrawal Assessment for Alcohol, Revised (CIWA-Ar), Drug Abuse Potential Scale (DAPS)

### Initial Telephone Screening

Subject eligibility is first assessed via telephone screening and a preliminary medical record review. Telephone screening occurs by experienced research personnel adept with this process. If the subject seems to be a likely candidate for inclusion in this protocol, he or she is invited to a screening visit for study eligibility.

### Screening Visit

At the screening visit, the research team explains the overall study plan to potential subjects and go over the consent form. Subjects have an initial screening evaluation that includes psychiatric history, medical history, mental status examination, physical examination, and laboratory assessment. Other detailed procedures are described in Table 13. If the examination and test results are acceptable and the subject meets the inclusion/exclusion criteria, the subject is invited to the baseline visit.

### Baseline PET Scan

The subjects receive a baseline PET scan before receiving study medications.

### Three Test Days (Receiving IM Ketamine (or Saline)+IM Naltrexone, Table 14)

TABLE 14
Three Test Days (receiving IM ketamine (or saline) + IM naltrexone)

| Time | Procedures |
|---|---|
| T − 60 min | Vital signs (blood pressure, pulse) |
| | Urine Toxicology/Pregnancy |
| | VAS (craving), MADRS, QIDS-SR, |
| | VAS (mood), HAM-A, BPRS, CADSS |
| T0 | IM ketamine (or saline) + IM naltrexone administered |
| T + 60 min | Vital signs, VAS (craving), MADRS, |
| | QIDS-SR, VAS (mood), HAM-A, |
| | BPRS, CADSS, DAPS |
| T + 120 min | PET scan |
| T + 240 min | Vital signs, VAS (craving), MADRS, |
| | QIDS-SR, VAS (mood), HAM-A, |
| | BPRS, CADSS |

### Outcome Measures

Primary outcome measure: (1) brain μ-opioid receptor occupancy (%).

Exploratory outcome measures: (2) MDD: Clinical response in symptoms of MDD as measured by the Montgomery-Asberg Depression Rating Scale (MADRS) (response defined as a ≥50% improvement from baseline in MADRS score). (3) SUD: Substance use as measured by the Time Line Follow Back (TLFB). (4) Remission in symptoms of MDD as measured by the MADRS (remission defined as a MADRS score≤9). (5) Primary substance craving as measured by the VAS (craving). (6) Abuse potential of the exemplary NMDA receptor negative allosteric modulator as measured by the Drug Abuse Potential Scale.

### Example 5: Intravenous Coformulation

This is a within-subject, randomized, double-blind, placebo-controlled crossover trial evaluating intravenous coformulation of an exemplary NMDA receptor antagonist (ketamine) and an exemplary opioid receptor antagonist (naltrexone) in treating major depressive disorder (MDD) and substance use disorder (SUD). It is evaluated whether intravenous coformulation of ketamine and naltrexone is safe and effective in reducing depression, substance use, and the abuse liability of ketamine. The study is described as follows.

### Research Design

A human laboratory study is conducted to test whether intravenous (IV) coformulation of ketamine and naltrexone is safe and effective for treating comorbid MDD and SUD (n=40). Particularly, this study evaluates whether placebo (midazolam 0.03 mg/kg+naltrexone 0 mg) and 3 different doses of IV naltrexone (25 mg vs. 50 mg vs. 100 mg)+ketamine (0.5 mg/kg) have different antidepressant effects (Table 3).

TABLE 15
Intramuscular Coformulation Research Design

| | | 4 Test Conditions (randomly assigned on 4 test days) |
|---|---|---|
| 1 | Placebo | IV Midazolam 0.03 mg/kg + IV Naltrexone 0 mg |
| 2 | Low dose | IV Ketamine 0.5 mg/kg + IV Naltrexone 25 mg |
| 3 | Medium | IV Ketamine 0.5 mg/kg + IV Naltrexone 50 mg |
| | dose | |
| 4 | High dose | IV Ketamine 0.5 mg/kg + IV Naltrexone 100 mg |

A within-subject, randomized, double-blind, placebo-controlled crossover trial is proposed, in which each subject completes 1 screening day and 4 test days (Visit 1 for screening, Visit 2 for Test Day #1, Visit 3 for Test Day #2, Visit 3 for Test Day #3, Visit 4 for Test Day #4). The 4 test conditions are randomly assigned on 4 test days. On 4 test days, subjects randomly receive (1) placebo (IV Midazolam 0.03 mg/kg+IV Naltrexone 0 mg), (2) IV Ketamine 0.5 mg/kg+IV Naltrexone 25 mg, (3) IV Ketamine 0.5 mg/kg+IV Naltrexone 50 mg, or (4) IV Ketamine 0.5 mg/kg+IV Naltrexone 100 mg in a crossover design. Each test day are separated at least 10 days apart.

### Study Subjects

A total of 40 subjects with MDD and SUD are enrolled into the study. Subjects are recruited through clinical referrals, flyers, research websites, online advertising, research boards, and posters. Subjects are identified via their response to advertisements and/or internal recruiting through research clinics and other health clinics. Subjects are asked to call at the number provided on the flyers if they are interested in participating in the research study. Patients were recruited in outpatient settings as they met the following criteria.

Inclusion Criteria: (1) Male or female, 21-65 years old; (2) Current major depressive disorder without psychotic features by DSM-5; (3) Montgomery-Asberg Depression Rating Scale (MADRS)≥20; (4) Current substance use disorder by DSM-5; (5) Abstinence from substance (e.g., alcohol, cocaine, opioid) for >5 days prior to ketamine injection; (6) Able to provide written informed consent.

Exclusion Criteria: (1) Current or past history of psychotic features or psychotic disorder; (2) Current or past history of delirium or dementia; (3) Current uncontrolled hypertension (systolic BP>170 mm Hg or diastolic BP>100 mm Hg); (4) Unstable medical condition or allergy to ketamine or naltrexone, as clinically determined by a physician; (5) Imminent suicidal or homicidal risk; (6) Pregnant or nursing women, positive pregnancy test, or inadequate birth control methods in women of childbearing potential; (7) Positive opioid drug screen test; (8) Opioid use within 10 days prior to study medication (injectable naltrexone); (9) Liver enzymes that are three times higher than the upper limit of normal.

### Procedures

Each subject completes 1 screening day and 4 test days (Visit 1 for screening, Visit 2 for Test Day #1, Visit 3 for Test Day #2, Visit 3 for Test Day #3, Visit 4 for Test Day #4). On 4 test days, subjects randomly receive (1) placebo (IV Midazolam 0.03 mg/kg+IV Naltrexone 0 mg), (2) IV Ketamine 0.5 mg/kg+IV Naltrexone 25 mg, (3) IV Ketamine 0.5 mg/kg+IV Naltrexone 50 mg, or (4) IV Ketamine 0.5 mg/kg+IV Naltrexone 100 mg in a crossover design (Table 4). Each test day is separated at least 10 days apart. All subjects receive usual standard care during this trial.

TABLE 16
Procedures involved from screening visit and 4 test days

| | | | Visit 3 | Visit 4 | Visit 5 |
|---|---|---|---|---|---|
| | | Visit 2 | Test | Test | Test |
| | Visit 1 | Randomized | Day | Day | Day |
| Procedures | Screening | Test Day #1 | #2 | #3 | #4 |
| IV ketamine (or | | X | X | X | X |
| midazolam) + | | | | | |
| IV naltrexone | | | | | |
| Informed Consent | X | | | | |
| Inclusion/Exclusion | X | | | | |
| Demographics | X | | | | |
| MINI | X | | | | |
| FHAM | X | | | | |
| Psychiatric | X | | | | |
| Evaluation | | | | | |
| Physical | X | | | | |
| Examination | | | | | |
| Liver Profile, GGT | X | | | | |
| CBC, Chemistry 7 | X | | | | |
| Urine Tox/Preg | X | X | X | X | X |
| Urinalysis | X | | | | |
| BP, Pulse, Weight | X | X | X | X | X |
| MADRS, QIDS-SR, | X | X | X | X | X |
| HAM-A | | | | | |
| C-SSRS | X | X | X | X | X |
| VAS (craving) | X | X | X | X | X |
| BPRS, CADSS, | | X | X | X | X |
| VAS (mood) | | | | | |
| Concomitant Meds | X | X | X | X | X |
| TLFB | X | X | X | X | X |
| CIWA-Ar | X | X | X | X | X |
| Breathalyzer | X | X | X | X | X |
| Adverse Event | | X | X | X | X |
| DAPS | | X | X | X | X |

### Abbreviations:

Mini International Neuropsychiatric Interview (MINI); Family History Assessment Module (FHAM), Columbia-Suicide Severity Rating Scale (C-SSRS), Montgomery-Åsberg

Depression Rating Scale (MADRS), Quick Inventory of Depressive Symptomatology-Self Report (QIDS-SR), Hamilton Anxiety Rating Scale (HAM-A), Time Line Follow Back (TLFB), Brief Psychiatric Rating Scale (BPRS), Clinician-Administered Dissociative States Scale (CADSS), Visual Analog Scale (VAS) of Mood States, Complete Blood Count (CBC), Visual Analog Scale (VAS) of Craving, Clinical Institute Withdrawal Assessment for Alcohol, Revised (CIWA-Ar), Drug Abuse Potential Scale (DAPS)

### Initial Telephone Screening

Subject eligibility is first assessed via telephone screening and a preliminary medical record review. Telephone screening occurs by experienced research personnel adept with this process. If the subject seems to be a likely candidate for inclusion in this protocol, he or she is invited to a screening visit for study eligibility.

### Screening Visit

At the screening visit, the research team explains the overall study plan to potential subjects and go over the consent form. Subjects have an initial screening evaluation that includes psychiatric history, medical history, mental status examination, physical examination, and laboratory assessment. Other detailed procedures are described in Table 16. If the examination and test results are acceptable and the subject meets the inclusion/exclusion criteria, the subject is invited to the baseline visit.

Four Test Days (receiving IV ketamine (or midazolam)+IV naltrexone, Table 5)

TABLE 17
Four Test Days (receiving IV ketamine (or midazolam) + IV naltrexone)

| Time | Procedures |
|---|---|
| T − 60 min | Vital signs (blood pressure, pulse) |
| | Urine Toxicology/Pregnancy |
| | VAS (craving), MADRS, QIDS-SR, VAS |
| | (mood), HAM-A, BPRS, CADSS* |
| T0 | IV ketamine (or midazolam) + IV naltrexone administered |
| T + 60 min | Vital signs, VAS (craving), MADRS, |
| | QIDS-SR, VAS (mood), HAM-A, |
| | BPRS, CADSS, DAPS |
| T + 240 min | Vital signs, VAS (craving), MADRS, |
| | QIDS-SR, VAS (mood), HAM-A, |
| | BPRS, CADSS |

If urine toxicology is positive for alcohol or illicit drugs (except marijuana), IV ketamine (or midazolam)+IV naltrexone administration session is cancelled and rescheduled. *Items that could not change within a session (e.g., sleep, appetite) will be rated only at T-60 and at follow-up sessions. After the subject receives study medication, he or she receives lunch and is medically cleared before leaving. If necessary, intravenous lorazepam (ativan) at 1 mg is available to reduce the behavioral effects of ketamine. This is determined by a research physician. Subjects are not permitted to drive to and from the hospital on medication treatment days, and subjects must make their own arrangements for a ride. If this is not possible, the study organizers provide transportation for the subject.

### Measures

Medical Assessments: Physical examination (including vital sign determination) and clinical laboratories are completed at the first visit. Urine toxicology screen and breathalyzer are performed on the morning of each infusion day, and the results are determined before proceeding with the infusions. The subject does not receive study medications on the day if urine toxicology results are positive for any illicit drugs (except marijuana) or if his/her breathalyzer is positive for alcohol. A pregnancy test is also administered to all reproductive age females enrolled in the study prior to participation.

Psychiatric Assessments: Ratings were performed by trained research assistants. Administration of clinical measures was supervised by the primary investigator. Each of the psychiatric assessment instruments is briefly described below. (1) Mini International Neuropsychiatric Interview (MINI): The Mini International Neuropsychiatric Interview (MINI) will be used to classify subjects according to the presence or absence of psychiatric disorders based on the DSM-5 criteria. (2) Montgomery-Asberg Depression Rating Scale (MADRS): The MADRS is a standardized instrument to ascertain depressed mood and neurovegetative signs and symptoms of a major depressive episode. (3) Quick Inventory of Depressive Symptomatology-Self Report (QIDS-SR): The QIDS-SR is a patient-rated depression instrument. (4) Hamilton Anxiety Rating Scale (HAM-A): The HAM-A is a standardized instrument to evaluate anxiety severity. (5) Brief Psychotic Rating Scale (BPRS): The BPRS is a standardized instrument that contains scales assessing psychotic symptoms including positive and negative symptoms, activation and emotional distress. (6) Clinician-Administered Dissociative States Scale (CADSS): The CADSS has self and interviewer-administered items including 5 subscales, generated a priori, evaluating dissociation including altered environmental perception, time perception, spatial/body perception, derealization and memory impairment. (7) Visual Analog Scale (VAS) of Mood States: The VAS includes scales for anxiety, drowsiness, high irritability, anger and sadness. The scales are 100 mm lines marked by subjects at a point corresponding to the apparent intensity of the feeling state (0)=none, to 100=most ever). (8) Visual Analog Scale (VAS) of craving: The scales are 100 mm lines marked by subjects at a point corresponding to the apparent intensity of craving for their primary drug (0=none, to 100=most ever). (9) Time Line Follow Back (TLFB): The TLFB is a standardized measure utilized for collecting information on daily alcohol use as well as other substance use. (10) Columbia-Suicide Severity Rating Scale (C-SSRS): The C-SSRS has both lifetime/recent and since last visit versions. The “Lifetime/Recent” version gathers information on lifetime history of suicidality and recent suicidal ideation/self-injurious behavior. The “Since Last Visit” version of the C-SSRS asks about any suicidal thoughts or behaviors the subject has exhibited since the last time administered the C-SSRS. (11) Clinical Institute Withdrawal Assessment for Alcohol, Revised (CIWA-Ar): The CIWA-Ar is a reliable and valid 10-item assessment for monitoring alcohol withdrawal symptoms. (12) Drug Abuse Potential Scale (DAPS): The DAPS will be used to assess abuse liability of study drugs.

### Outcome Measures

Primary outcome measure: (1) MDD: Clinical response in symptoms of MDD as measured by the Montgomery-Asberg Depression Rating Scale (MADRS) (response defined as a ≥50% improvement from baseline in MADRS score). (2) SUD: Substance use as measured by the Time Line Follow Back (TLFB).

Exploratory outcome measures: (3) Remission in symptoms of MDD as measured by the MADRS (remission defined as a MADRS score≤9). (4) Primary substance craving as measured by the VAS (craving). (5) Abuse potential of ketamine as measured by the Drug Abuse Potential Scale.

### Example 6: Treatment of Comorbid Major Depression Disorder (MDD) and Alcohol

### Use Disorder (AUD)

This is a within-subject, randomized, double-blind, placebo-controlled crossover trial evaluating intranasal coformulation of an exemplary NMDA receptor antagonist (ketamine) and an exemplary opioid receptor antagonist (naltrexone) in treating comorbid major depressive disorder (MDD) and alcohol use disorder (AUD). It was evaluated whether repeated ketamine treatment and naltrexone is safe and effective in reducing depression, substance use, and the abuse liability of ketamine. The study is described as follows.

### Research Design

A 8-week, randomized, double-blind, placebo-controlled trial has been conducted. It included 60 veterans with comorbid MDD and AUD to test safety and efficiency of repeated ketamine treatment (0.5 mg/kg: once a week for 4 weeks: a total of 4 ketamine infusions) plus naltrexone with a follow-up of 4 weeks and a 4 month follow up. 3 Groups including ketamine and naltrexone, ketamine alone, and placebo have been compared for treating MDD and AUD as shown Table 18.

TABLE 18
Three groups of testing safety and efficiency of ketamine and naltrexone

| Group | Treatments |
|---|---|
| 1 | IV ketamine (0.5 mg/kg) + IM naltrexone (380 mg) (n = 20) |
| 2 | IV ketamine (0.5 mg/kg) + IM Placebo (n = 20) |
| 3 | IV placebo (midazolam 0.045 mg/kg) + IM Placebo (n = 20) |

### Study Subject

A total of 65 depressed and recently-detoxified alcohol dependent subjects between the ages of 21-65 were recruited. Subjects were accepted into protocol after an opportunity to review and provide voluntary written informed consent and completion of a comprehensive medical and psychiatric history, physical examination, mental status examination, and routine laboratory assessment. Patients were recruited in outpatient settings. Participants were enrolled if they meet the following criteria.

### Inclusion Criteria:

1. Male or female veterans, 21-65 years old
2. Current major depressive disorder without psychotic features by DSM-5 (antidepressant regimens can be allowed and changed during the trial)
3. Montgomery-Asberg Depression Rating Scale (MADRS)≥20
4. A minimum of 4 of 11 current alcohol use disorder symptoms by DSM-5
5. Heavy drinking at least 4 times in the past month (‘heavy drinking’ defined as ≥5 standard drinks per day for men and >4 standard drinks per day for women) 6 Able to provide written informed consent

### Exclusion Criteria:

1. Current substance use disorder by DSM-5 in the past 3 months (except alcohol, tobacco, or cannabis)
2. Current or past history of psychotic features or psychotic disorder
3. Current dementia
4. Current uncontrolled hypertension (systolic BP>170 mm Hg or diastolic BP>100 mm Hg)
5. Unstable medical condition or allergy to ketamine, midazolam, naltrexone, or lorazepam—clinically determined by a physician
6. Imminent suicidal or homicidal risk
7. Pregnant or nursing women, positive pregnancy test, or inadequate birth control methods in women of childbearing potential
8. Positive opioid or illicit drug screen test (except marijuana)
9. Opioid use within 10 days prior to study medication (injectable naltrexone) or risks for opioid use during the study
10. Liver enzymes that are three times higher than the upper limit of normal
11. Current use of benzodiazepine
12. Acute narrow-angle glaucoma

### Procedures

Each subject completes the following procedures. Visit 1 for screening, Visit 2 for IM naltrexone or placebo, Visits 3 to 6 for administration of ketamine or midazolam, Visit 7 for IM naltrexone or placebo, and from Visit 8 for additional tests. Each test day will be separated at least 7 days apart.

TABLE 19
Procedures involved from screening visit, administration visit, and test days

| | 1 | | | | | | | | | | 4-mo |
|---|---|---|---|---|---|---|---|---|---|---|---|
| Visit | Screening | 2 | 3 | 4 | 5 | 6 | 7 | 8 | 9 | 10 | F/U |
| Day | −30-0 | −9-1 | 0+ | 7+ | 14+ | 21+ | 28+ | 35+ | 42+ | 49+ | 120+ |
| IM naltrexone | | X | | | | | X | | | | |
| or placebo | | | | | | | | | | | |
| Ketamine or | | | X | X | X | X | | | | | |
| Midazolam | | | | | | | | | | | |
| Informed | X | | | | | | | | | | |
| Consent | | | | | | | | | | | |
| Inclusion/ | X | | | | | | | | | | |
| Exclusion | | | | | | | | | | | |
| Demo- | X | | | | | | | | | | |
| graphics | | | | | | | | | | | |
| SCID | X | | | | | | | | | | |
| FHAM | X | | | | | | | | | | |
| Psychiatric | X | | | | | | | | | | |
| Eval | | | | | | | | | | | |
| Physical | X | | | | | | | | | | |
| Exam | | | | | | | | | | | |
| EKG | X | | | | | | | | | | |
| EtG, Liver | X | | | | | X | | | | X | X |
| Profile, GGT | | | | | | | | | | | |
| CBC, | X | | | | | X | | | | | |
| Chemistry 7 | | | | | | | | | | | |
| Urine | X | | X | X | X | X | X | | | | |
| Tox/Preg | | | | | | | | | | | |
| Urinalysis | X | | | | | | | | | | |
| BP, Pulse, | X | | X | X | X | X | X | X | X | X | X |
| Weight | | | | | | | | | | | |
| MADRS, | X | | X | X | X | X | X | X | X | X | X |
| QIDS-SR, | | | | | | | | | | | |
| HAM-A | | | | | | | | | | | |
| C-SSRS | X | | X | X | X | X | X | X | X | X | X |
| YCS, OCDS | X | | X | X | X | X | X | X | X | X | X |
| BPRS, | | | X | X | X | X | | | | | |
| CADSS, | | | | | | | | | | | |
| VAS | | | | | | | | | | | |
| Concomitant | X | | X | X | X | X | X | X | X | X | X |
| Meds | | | | | | | | | | | |
| TLFB | X | | X | X | X | X | X | X | X | X | X |
| CIWA-Ar | X | | X | X | X | X | X | X | X | X | X |
| Breathalyzer | X | | X | X | X | X | X | X | X | X | X |
| Adverse | | | X | X | X | X | X | X | X | X | |
| Event | | | | | | | | | | | |
| DAPS | | | X | X | X | X | X | X | X | X | X |
| CGI | | | X | X | X | X | X | X | X | X | X |

### Montgomery-Asberg Depression Rating Scale (MADRS)

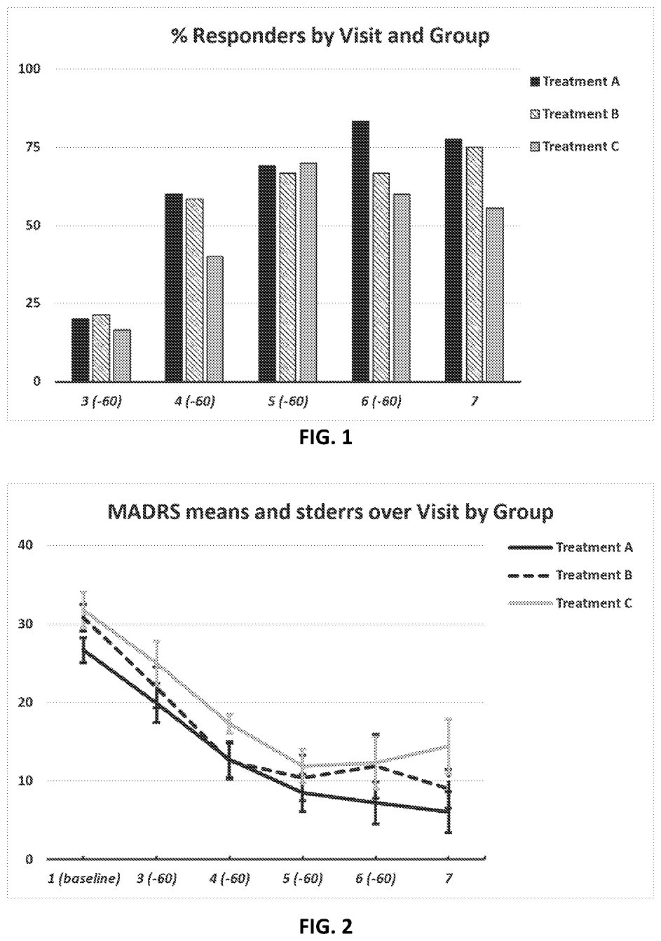

Primary outcomes of this test was % of responders that was defined as more than 50% reduction from a baseline. The subjects of each group were treated by three different treatments as shown in Table 18. Clinical response at visit 3 (day 0 after 1st ketamine infusion, 240 minutes after starting ketamine infusion (T+240)) and visit 6 (day 21 after 4th ketamine infusion, 240 minutes after starting ketamine infusion (T+240)) in symptoms of MDD as measured by the MADRS (response defined as a ≥50% improvement from baseline (visit 1) in MADRS score). As shown in FIG. 1, Group 1 treated by IV ketamine (0.5 mg/kg) and IM naltrexone (380 mg) showed higher percentages of responders per visit compared with Group 2 and Group 3 treated by either IV ketamine (0.5 mg/kg) and IM Placebo or only Placebos.

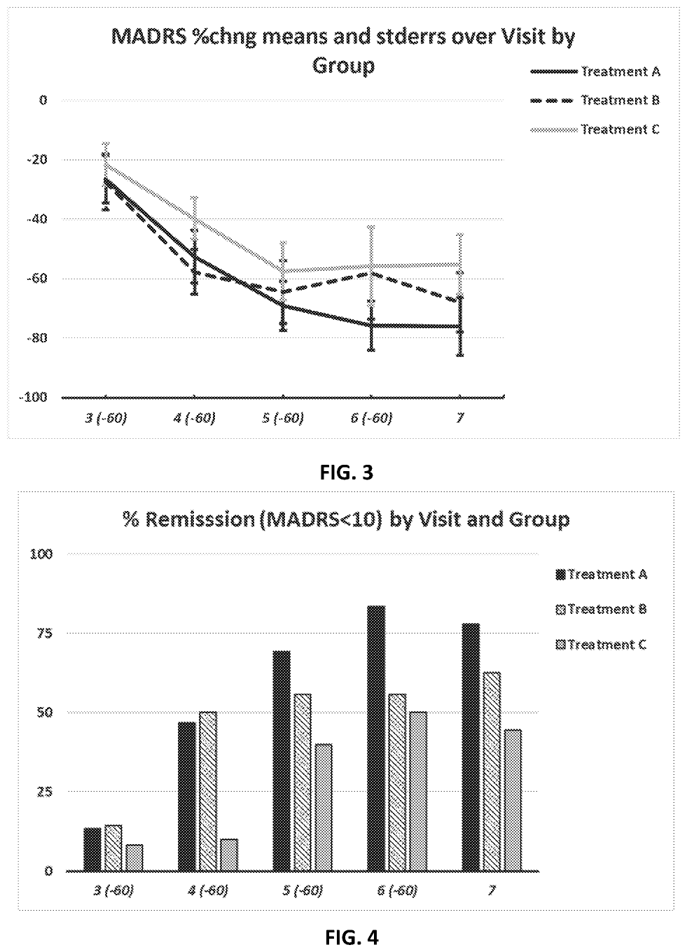

Continuous scores on MADRS were measured for the same Groups per visit. As shown in FIG. 2, Group 1 treated by IV ketamine (0.5 mg/kg) and IM naltrexone (380 mg) showed lower level of MADRS per visit compared with Group 2 and Group 3 treated by either IV ketamine (0.5 mg/kg) and IM Placebo or only Placebos. FIG. 3 also shows changes of MADRS from a baseline for each Group per visit.

MADRS remission has been defined as MADRS is less than 10 and measured for the same Group per visit. As shown in FIG. 4, Group 1 treated by IV ketamine (0.5 mg/kg) and IM naltrexone (380 mg) showed higher percentages of remission per visit compared with Group 2 and Group 3 treated by either IV ketamine (0.5 mg/kg) and IM Placebo or only Placebos.

### Data Analysis of Participants System (DAPS)-Drug Liking

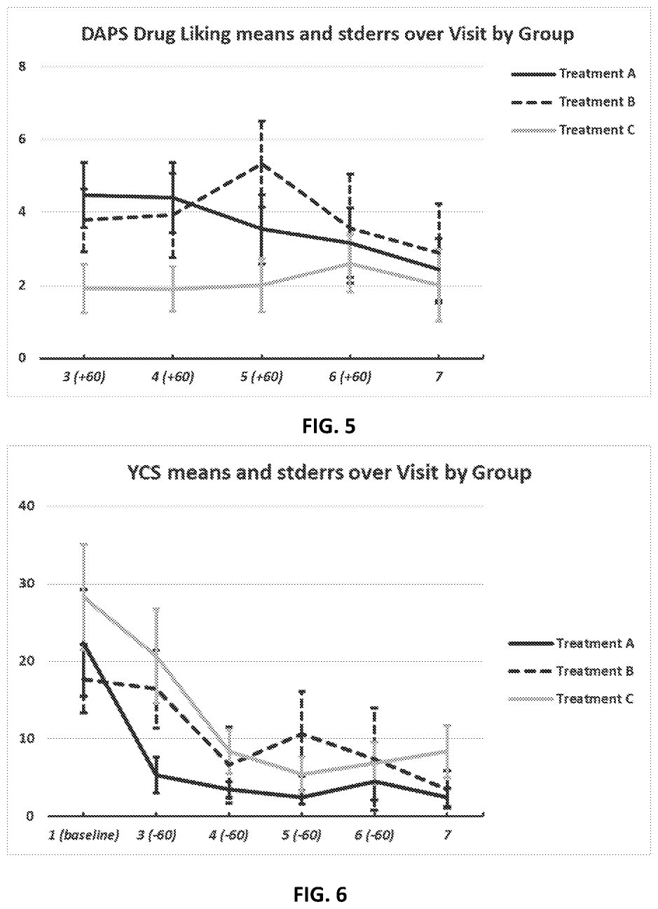

DAPS data has a scale of 0 to 10 with anchors: 0 represents “does not like”, 5 represents “moderately likes”, and 10 represents “extremely likes”. DAPS data is not collected at visit 1 (baseline) and collected at visits 3-11 (data of visits 8-11 are not shown here). On infusion days (visits 3-6), the DAPS is only collected once at the 60 minutes after the infusion. FIG. 5 shows the resulting data that drug liking was treated as being continuous. Group 1 treated by IV ketamine (0.5 mg/kg) and IM naltrexone (380 mg) showed mostly lower level of DAPS drug liking per visit compared with Group 2 treated by IV ketamine (0.5 mg/kg) and IM Placebo. Group 3 treated by only Placebos exhibited low levels of DAPS drug liking.

### The Yale Carving Scale (YCS) Craving

A subject marks his/her “desire for an alcoholic beverage right now” on a horizontal line 112 mm in length, with the anchor at the following distance from the left in mm: 0 represent “no sensation”, 2 represents “barely noticeable”, 7 represents “weak”, 19 represents “moderate”, 38 represents “strong”, 60 represents “very strong”, and 112 represents “strongest imaginable kind”. YCS was collected at visit 1 (baseline), and then visits 3-11. FIG. 6 show results of visit 1, visits 3-6 (infusion days), and visit 7. On infusion days, the YCS was collected at 60 minutes before the infusion to capture effects of the prior infusion (FIG. 6). As shown in FIG. 6, Group 1 showed lower level of YCS values than Group 2 and Group 3

While illustrative embodiments of the present application have been shown and described herein, it will be obvious to those skilled in the art that such embodiments are provided by way of example only. Numerous variations, changes, and substitutions will now occur to those skilled in the art without departing from the present application. It should be understood that various alternatives to the embodiments of the present application described herein may be employed in practicing the present application. It is intended that the following claims define the scope of the present application and that methods and structures within the scope of these claims and their equivalents be covered thereby.

### Enumerated Embodiments

The following exemplary embodiments are provided, the numbering of which is not to be construed as designating levels of importance.

Embodiment 1 provides a pharmaceutical composition comprising a N-methyl-D-aspartate receptor modulator and a μ-opioid receptor modulator, wherein the μ-opioid receptor modulator is present in an amount that, when administered to a subject, occupies at least 10% of the subject's μ-opioid receptors in vivo as measured by positron emission tomography with a [¹¹C]-carfentanil ligand.

Embodiment 2 provides the pharmaceutical composition of Embodiment 1, wherein the μ-opioid receptor modulator is present in an amount that, when administered to a subject, occupies at least 25% of the subject's μ-opioid receptors in vivo.

Embodiment 3 provides the pharmaceutical composition of any one of Embodiments 1-2, wherein the μ-opioid receptor modulator is present in an amount that, when administered to a subject, occupies at least 50% of the subject's μ-opioid receptors in vivo.

Embodiment 4 provides the pharmaceutical composition of any one of Embodiments 1-3, wherein the μ-opioid receptor modulator is present in an amount that, when administered to a subject, occupies at least 75% of the subject's μ-opioid receptors in vivo.

Embodiment 5 provides the pharmaceutical composition of any one of Embodiments 1-4, wherein the μ-opioid receptor modulator is present in an amount that, when administered to a subject, occupies at least 90% of the subject's μ-opioid receptors in vivo.

Embodiment 6 provides the pharmaceutical composition of any one of Embodiments 1-5, wherein the μ-opioid receptor modulator is present in an amount that, when administered to a subject, occupies at least 99% of the subject's μ-opioid receptors in vivo.

Embodiment 7 provides the pharmaceutical composition of any one of Embodiments 1-6, wherein the N-methyl-D-aspartate receptor modulator is selected from the group consisting of a N-methyl-D-aspartate receptor antagonist, a N-methyl-D-aspartate receptor negative allosteric modulator, and a N-methyl-D-aspartate receptor partial agonist.

Embodiment 8 provides the pharmaceutical composition of any one of Embodiments 1-7, wherein the N-methyl-D-aspartate receptor modulator comprises a compound selected from the group consisting of ketamine, R-ketamine, S-ketamine, nitrous oxide, memantine, amantadine, racemic dextromethorphan, dextromethorphan, lanicemine, phencyclidine, dizocilpine, CERC-301, CGP 37849, 1-aminocylopropanecarboxylic acid, traxoprodil, Ro 25-6981, eliprodil, methoxetamine, CPPene, AP5, AP7, Selfotel (CGS-19755), minocycline, nitromemantine, PD-137889, rolicyclidine, tenocyclidine, methoxydine, tiletamine, neramexane, etoxadrol, dexoxadrol, WMS-2539, NEFA, remacemide, 3-MeO-PCP, 8A-PDHQ, atomoxetine, AZD6765, agmatine, chloroform, delucemine, dextrallorphan, dextrorphan, diphenidine, eticyclidine, gacyclidine, aptiganel, HU-211, huperzine A, dipeptide D-Phe-L-Tyr, ibogaine, rhynchophylline, rapastinel, NRX-1074, 7-Chlorokynurenic acid, 4-Chlorokynurenine, 5,7-Dichlorokynurenic acid, Kynurenic acid, TK-40, L-Phenylalanine, xenon, methadone, EU1180-438, radiprodil, Ifenprodil, TCN-201, MPX-004, MPX-007, NAB-14, EVT-101, QNZ-46, DQP-1105, pregnanolone sulfate (3α5βS), UBP608, UBP618, UBP551, UBP512, HA-966, felbamate, PEAQX (NVP-AAM077), PD0196860, RGH896, MK0657, L701324, LY293558, LY300164, LY246492, LY202157, NYX-783, and/or any salt, solvate, enantiomer, tautomer, stereoisomer, and/or geometric isomer thereof, and/or any mixtures thereof.

Embodiment 9 provides the pharmaceutical composition of any one of Embodiments 1-8, wherein the N-methyl-D-aspartate receptor modulator comprises ketamine, and/or any salt, solvate, enantiomer, tautomer, stereoisomer, and/or geometric isomer thereof, and/or any mixtures thereof.

Embodiment 10 provides the pharmaceutical composition of any one of Embodiments 1-9, wherein the N-methyl-D-aspartate receptor modulator is present in an amount of about 10 mg to about 60 mg.

Embodiment 11 provides the pharmaceutical composition of any one of Embodiments 1-10, wherein the N-methyl-D-aspartate receptor modulator is present in an amount of about 15 mg to about 95 mg.

Embodiment 12 provides the pharmaceutical composition of any one of Embodiments 1-11, wherein the N-methyl-D-aspartate receptor modulator is present in an amount of about 50 mg.

Embodiment 13 provides the pharmaceutical composition of any one of Embodiments 1-12, wherein the μ-opioid receptor modulator is selected from the group consisting of a μ-opioid receptor antagonist, a μ-opioid receptor negative allosteric modulator, and a μ-opioid receptor partial agonist.

Embodiment 14 provides the pharmaceutical composition of any one of Embodiments 1-13, wherein the μ-opioid receptor modulator comprises a compound selected from the group consisting of naltrexone, naloxone, nalmefene, nalodeine, and/or any salt, solvate, enantiomer, tautomer, stereoisomer, and/or geometric isomer thereof, and/or any mixtures thereof.

Embodiment 15 provides the pharmaceutical composition of Embodiment 14, wherein the naltrexone is formulated for extended-release (long-acting naltrexone) which prolongs the serum half-life of the naltrexone.

Embodiment 16 provides the pharmaceutical composition of Embodiment 15, wherein the long-acting naltrexone is VIVITROL®.

Embodiment 17 provides the pharmaceutical composition of Embodiment 14, wherein the naloxone is formulated for extended-release (long-acting naloxone) which prolongs the serum half-life of the naloxone.

Embodiment 18 provides the pharmaceutical composition of Embodiment 14, wherein the nalmefene is formulated for extended-release (long-acting nalmefene) which prolongs the serum half-life of the nalmefene.

Embodiment 19 provides the pharmaceutical composition of any one of Embodiments 1-16, wherein the μ-opioid receptor modulator comprises naltrexone, and/or any salt, solvate, enantiomer, tautomer, stereoisomer, and/or geometric isomer thereof, and/or any mixtures thereof.

Embodiment 20 provides the pharmaceutical composition of any one of Embodiments 1-19, wherein the μ-opioid receptor modulator is present in an amount of lower than about 400 mg.

Embodiment 21 provides the pharmaceutical composition of any one of Embodiments 1-20, wherein the μ-opioid receptor modulator is present in an amount of about 300 mg to about 400 mg.

Embodiment 22 provides the pharmaceutical composition of any one of Embodiments 1-20, wherein the μ-opioid receptor modulator is present in an amount of about 200 mg to about 300 mg.

Embodiment 23 provides the pharmaceutical composition of any one of Embodiments 1-20, wherein the μ-opioid receptor modulator is present in an amount of about 100 mg to about 200 mg.

Embodiment 24 provides the pharmaceutical composition of any one of Embodiments 1-20, wherein the μ-opioid receptor modulator is present in an amount of about 5 mg to about 100 mg.

Embodiment 25 provides the pharmaceutical composition of any one of Embodiments 1-20, wherein the μ-opioid receptor modulator is present in an amount of about 25 mg to about 100 mg.

Embodiment 26 provides the pharmaceutical composition of any one of Embodiments 1-20, wherein the μ-opioid receptor modulator is present in an amount of about 1 mg to about 90 mg.

Embodiment 27 provides the pharmaceutical composition of any one of Embodiments 1-20, wherein the μ-opioid receptor modulator is present in an amount of about 2 mg to about 10 mg.

Embodiment 28 provides the pharmaceutical composition of any one of Embodiments 1-16 and 19-27, wherein the N-methyl-D-aspartate receptor modulator comprises ketamine, and/or any salt, solvate, enantiomer, tautomer, stereoisomer, and/or geometric isomer thereof, and/or any mixtures thereof; and wherein the μ-opioid receptor modulator comprises naltrexone, and/or any salt, solvate, enantiomer, tautomer, stereoisomer, and/or geometric isomer thereof, and/or any mixtures thereof.

Embodiment 29 provides the pharmaceutical composition of Embodiment 28, wherein the N-methyl-D-aspartate receptor modulator is present in an amount of about 10 mg to about 95 mg, and wherein the μ-opioid receptor modulator is present in an amount of about 10 mg to about 400 mg.

Embodiment 30 provides a method of treating, ameliorating, and/or preventing a disease or disorder in a subject in need thereof, the method comprising administering at least one of the following: (a) the pharmaceutical composition of any one of Embodiments 1-29 to the subject: (b) a N-methyl-D-aspartate receptor modulator and a μ-opioid receptor modulator, wherein the μ-opioid receptor modulator is administered in an amount that occupies at least 10% of the subject's μ-opioid receptors in vivo as measured by positron emission tomography with a [¹¹C]-carfentanil ligand.

Embodiment 31 provides the method of Embodiment 30, wherein the N-methyl-D-aspartate receptor modulator and the μ-opioid receptor modulator are administered concurrently.

Embodiment 32 provides the method of Embodiment 30, wherein the N-methyl-D-aspartate receptor modulator and the μ-opioid receptor modulator are administered separately.

Embodiment 33 provides the method of Embodiment 30, wherein the N-methyl-D-aspartate receptor modulator and the μ-opioid receptor modulator are administered sequentially.

Embodiment 34 provides the method of any one of Embodiments 30-33, wherein the N-methyl-D-aspartate receptor modulator is administered once per day, twice per day, or three times per day.

Embodiment 35 provides the method of any one of Embodiments 30-33, wherein the N-methyl-D-aspartate receptor modulator is administered once per week, twice per week, or three times per week.

Embodiment 36 provides the method of any one of Embodiments 30-33, wherein the N-methyl-D-aspartate receptor modulator is administered once per month, twice per month, or three times per month.

Embodiment 37 provides the method of any one of Embodiments 30-36, wherein the μ-opioid receptor modulator is administered once per day, twice per day, or three times per day.

Embodiment 38 provides the method of any one of Embodiments 30-36, wherein the μ-opioid receptor modulator is administered once per week, twice per week, or three times per week.

Embodiment 39 provides the method of any one of Embodiments 30-36, wherein the μ-opioid receptor modulator is administered once per month, twice per month, or three times per month.

Embodiment 40 provides the method of any one of Embodiments 30-39, wherein the N-methyl-D-aspartate receptor modulator is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

Embodiment 41 provides the method of any one of Embodiments 30-40, wherein the μ-opioid receptor modulator is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

Embodiment 42 provides the method of any one of Embodiments 30-41, wherein the disease or disorder is at least one selected from the group consisting of major depressive disorder, major depressive episode in bipolar disorder (bipolar depression), bipolar I disorder, bipolar II disorder, persistent depressive disorder (dysthymia), disruptive mood dysregulation disorder, major depressive disorder (including major depressive episode), premenstrual dysphoric disorder, substance/medication-induced depressive disorder, depressive disorder due to another medical condition, other specified depressive disorder, unspecified depressive disorder, anxiety disorder, generalized anxiety disorder, social anxiety disorder (social phobia), specific phobia, panic disorder, agoraphobia, separation anxiety disorder, selective mutism, substance-induced anxiety disorder, medication-induced anxiety disorder, anxiety disorder due to another medical condition, borderline personality disorder, treatment-resistant depression, unspecified anxiety disorder, and posttraumatic stress disorder.

Embodiment 43 provides the method of any one of Embodiments 30-42, wherein the subject suffers from a comorbid substance use disorder.

Embodiment 44 provides the method of any one of Embodiments 30-43, wherein administration of the μ-opioid receptor modulator prevents, ameliorates, and/or minimizes abuse of the N-methyl-D-aspartate receptor modulator and an abused substance that is not the N-methyl-D-aspartate receptor modulator.

Embodiment 45 provides the method of Embodiment 44, wherein the abused substance is selected from the group consisting of alcohol, a stimulant, an opioid, cannabis, a hallucinogen, an inhalant, a sedative, a hypnotic, an anxiolytic, tobacco, caffeine, nicotine, and other (unknown) substances.

Embodiment 46 provides the method of Embodiment 45, wherein the stimulant comprises cocaine or amphetamine.

Embodiment 47 provides the method of Embodiment 45, wherein the hallucinogen comprises lysergic acid diethylamide (LSD) and/or phencyclidine.

Embodiment 48 provides the method of Embodiment 45, wherein the anxiolytic comprises a barbiturate and/or a benzodiazepine.

Embodiment 49 provides the method of any one of Embodiments 30-48, wherein the administering has at least one effect selected from the group consisting of reduced anxiety, reduced irritability, reduced anger, and reduced alcohol consumption.

Embodiment 50 provides a method of treating, ameliorating, and/or preventing a disease or disorder in a subject in need thereof comprising administering to the subject a coformulation comprising a N-methyl-D-aspartate receptor modulator and a μ-opioid receptor modulator, wherein the coformulation is administered to the subject repeatedly.

Embodiment 51 provides the method of Embodiment 50, wherein the N-methyl-D-aspartate receptor modulator is selected from the group consisting of a N-methyl-D-aspartate receptor antagonist, a N-methyl-D-aspartate receptor negative allosteric modulator, and a N-methyl-D-aspartate receptor partial agonist.

Embodiment 52 provides the method of any one of Embodiments 50-51, wherein the N-methyl-D-aspartate receptor modulator comprises a compound selected from the group consisting of ketamine, R-ketamine, S-ketamine, nitrous oxide, memantine, amantadine, racemic dextromethorphan, dextromethorphan, lanicemine, phencyclidine, dizocilpine, CERC-301, CGP 37849, 1-aminocylopropanecarboxylic acid, traxoprodil, Ro 25-6981, eliprodil, methoxetamine, CPPene, AP5, AP7, Selfotel (CGS-19755), minocycline, nitromemantine, PD-137889, rolicyclidine, tenocyclidine, methoxydine, tiletamine, neramexane, etoxadrol, dexoxadrol, WMS-2539, NEFA, remacemide, 3-MeO-PCP, 8A-PDHQ, atomoxetine, AZD6765, agmatine, chloroform, delucemine, dextrallorphan, dextrorphan, diphenidine, eticyclidine, gacyclidine, aptiganel, HU-211, huperzine A, dipeptide D-Phe-L-Tyr, ibogaine, rhynchophylline, rapastinel, NRX-1074, 7-Chlorokynurenic acid, 4-Chlorokynurenine, 5,7-Dichlorokynurenic acid, Kynurenic acid, TK-40, L-Phenylalanine, xenon, methadone, EU1180-438, radiprodil, Ifenprodil, TCN-201, MPX-004, MPX-007, NAB-14, EVT-101, QNZ-46, DQP-1105, pregnanolone sulfate (3α5βS), UBP608, UBP618, UBP551, UBP512, HA-966, felbamate, PEAQX (NVP-AAM077), PD0196860, RGH896, MK0657, L701324, LY293558, LY300164, LY246492, LY202157, NYX-783, and/or any salt, solvate, enantiomer, tautomer, stereoisomer, and/or geometric isomer thereof, and/or any mixtures thereof.

Embodiment 53 provides the method of any one of Embodiments 50-52, wherein the N-methyl-D-aspartate receptor modulator comprises ketamine, and/or any salt, solvate, enantiomer, tautomer, stereoisomer, and/or geometric isomer thereof, and/or any mixtures thereof.

Embodiment 54 provides the method of any one of Embodiments 50-53, wherein the N-methyl-D-aspartate receptor modulator is present in an amount of about 10 mg to about 60 mg.

Embodiment 55 provides the method of any one of Embodiments 50-54, wherein the N-methyl-D-aspartate receptor modulator is present in an amount of about 15 mg to about 95 mg.

Embodiment 56 provides the method of any one of Embodiments 50-55, wherein the N-methyl-D-aspartate receptor modulator is present in an amount of about 50 mg.

Embodiment 57 provides the method of any one of Embodiments 50-56, wherein the μ-opioid receptor modulator is selected from the group consisting of a μ-opioid receptor antagonist, a μ-opioid receptor negative allosteric modulator, and a μ-opioid receptor partial agonist.

Embodiment 58 provides the method of any one of Embodiments 50-57, wherein the μ-opioid receptor modulator comprises a compound selected from the group consisting of naltrexone, naloxone, nalmefene, nalodeine, and/or any salt, solvate, enantiomer, tautomer, stereoisomer, and/or geometric isomer thereof, and/or any mixtures thereof.

Embodiment 59 provides the method of any one of Embodiments 50-58, wherein the μ-opioid receptor modulator comprises naltrexone, and/or any salt, solvate, enantiomer, tautomer, stereoisomer, and/or geometric isomer thereof, and/or any mixtures thereof.

Embodiment 60 provides the method of any one of Embodiments 50-59, wherein the μ-opioid receptor modulator is present in an amount of lower than about 400 mg.

Embodiment 61 provides the method of any one of Embodiments 50-60, wherein the μ-opioid receptor modulator is present in an amount of about 300 mg to about 400 mg.

Embodiment 62 provides the method of any one of Embodiments 50-60, wherein the μ-opioid receptor modulator is present in an amount of about 200 mg to about 300 mg.

Embodiment 63 provides the method of any one of Embodiments 50-60, wherein the μ-opioid receptor modulator is present in an amount of about 100 mg to about 200 mg.

Embodiment 64 provides the method of any one of Embodiments 50-60, wherein the μ-opioid receptor modulator is present in an amount of about 5 mg to about 100 mg.

Embodiment 65 provides the method of any one of Embodiments 50-60, wherein the μ-opioid receptor modulator is present in an amount of about 25 mg to about 100 mg.

Embodiment 66 provides the method of any one of Embodiments 50-60, wherein the μ-opioid receptor modulator is present in an amount of about 1 mg to about 90 mg.

Embodiment 67 provides the method of any one of Embodiments 50-60, wherein the μ-opioid receptor modulator is present in an amount of about 2 mg to about 10 mg.

Embodiment 68 provides the method of any one of Embodiments 50-67, wherein the coformulation is administered once per day, twice per day, or three times per day.

Embodiment 69 provides the method of any one of Embodiments 50-67, wherein the coformulation is administered once per week, twice per week, or three times per week.

Embodiment 70 provides the method of any one of Embodiments 50-67, wherein the coformulation is administered once per month, twice per month, or three times per month.

Embodiment 71 provides the method of any one of Embodiments 50-70, wherein the coformulation is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

Embodiment 72 provides the method of any one of Embodiments 50-71, wherein the disease or disorder is at least one selected from the group consisting of major depressive disorder, major depressive episode in bipolar disorder (bipolar depression), bipolar I disorder, bipolar II disorder, persistent depressive disorder (dysthymia), disruptive mood dysregulation disorder, major depressive disorder (including major depressive episode), premenstrual dysphoric disorder, substance/medication-induced depressive disorder, depressive disorder due to another medical condition, other specified depressive disorder, unspecified depressive disorder, anxiety disorder, generalized anxiety disorder, social anxiety disorder (social phobia), specific phobia, panic disorder, agoraphobia, separation anxiety disorder, selective mutism, substance-induced anxiety disorder, medication-induced anxiety disorder, anxiety disorder due to another medical condition, borderline personality disorder, treatment-resistant depression, unspecified anxiety disorder, and posttraumatic stress disorder.

Embodiment 73 provides the method of any one of Embodiments 50-72, wherein the subject suffers from a comorbid substance use disorder.

Embodiment 74 provides the method of any one of Embodiments 50-73, wherein presence of the μ-opioid receptor modulator prevents, ameliorates, and/or minimizes abuse of the N-methyl-D-aspartate receptor modulator and an abused substance that is not the N-methyl-D-aspartate receptor modulator.

Embodiment 75 provides the method of Embodiment 74, wherein the abused substance is selected from the group consisting of alcohol, a stimulant, an opioid, cannabis, a hallucinogen, an inhalant, a sedative, a hypnotic, an anxiolytic, tobacco, caffeine, nicotine, and other (unknown) substances.

Embodiment 76 provides the method of Embodiment 75, wherein the stimulant comprises cocaine and/or amphetamine.

Embodiment 77 provides the method of Embodiment 75, wherein the hallucinogen comprises lysergic acid diethylamide (LSD) and/or phencyclidine.

Embodiment 78 provides the method of Embodiment 75, wherein the anxiolytic comprises a barbiturate and/or a benzodiazepine.

Embodiment 79 provides the method of any one of Embodiments 50-78, wherein the administering has at least one effect selected from the group consisting of reduced anxiety, reduced irritability, reduced anger, and reduced alcohol consumption

### INCORPORATION BY REFERENCE

All publications, patents, and patent applications mentioned in this specification are herein incorporated by reference to the same extent as if each individual publication, patent, or patent application was specifically and individually indicated to be incorporated by reference.

1. A pharmaceutical composition comprising a N-methyl-D-aspartate receptor modulator and a μ-opioid receptor modulator,
wherein the μ-opioid receptor modulator is present in an amount that, when administered to a subject, occupies at least 10% of the subject's μ-opioid receptors in vivo as measured by positron emission tomography with a [¹¹C]-carfentanil ligand.

2. The pharmaceutical composition of claim 1, wherein the μ-opioid receptor modulator is present in an amount that, when administered to a subject, occupies at least 25%, at least 50%, at least 75%, at least 90%, or at least 99% of the subject's μ-opioid receptors in vivo.

3-6. (canceled)

7. The pharmaceutical composition of claim 1, wherein the N-methyl-D-aspartate receptor modulator is selected from the group consisting of a N-methyl-D-aspartate receptor antagonist, a N-methyl-D-aspartate receptor negative allosteric modulator, and a N-methyl-D-aspartate receptor partial agonist.

8. The pharmaceutical composition of claim 1, wherein the N-methyl-D-aspartate receptor modulator comprises a compound selected from the group consisting of ketamine, R-ketamine, S-ketamine, nitrous oxide, memantine, amantadine, racemic dextromethorphan, dextromethorphan, lanicemine, phencyclidine, dizocilpine, CERC-301, CGP 37849, 1-aminocylopropanecarboxylic acid, traxoprodil, Ro 25-6981, eliprodil, methoxetamine, CPPene, AP5, AP7, Selfotel (CGS-19755), minocycline, nitromemantine, PD-137889, rolicyclidine, tenocyclidine, methoxydine, tiletamine, neramexane, etoxadrol, dexoxadrol, WMS-2539, NEFA, remacemide, 3-MeO-PCP, 8A-PDHQ, atomoxetine, AZD6765, agmatine, chloroform, delucemine, dextrallorphan, dextrorphan, diphenidine, eticyclidine, gacyclidine, aptiganel, HU-211, huperzine A, dipeptide D-Phe-L-Tyr, ibogaine, rhynchophylline, rapastinel, NRX-1074, 7-Chlorokynurenic acid, 4-Chlorokynurenine, 5,7-Dichlorokynurenic acid, Kynurenic acid, TK-40, L-Phenylalanine, xenon, methadone, EU1180-438, radiprodil, Ifenprodil, TCN-201, MPX-004, MPX-007, NAB-14, EVT-101, QNZ-46, DQP-1105, pregnanolone sulfate (3α5βS), UBP608, UBP618, UBP551, UBP512, HA-966, felbamate, PEAQX (NVP-AAM077), PD0196860, RGH896, MK0657, L701324, LY293558, LY300164, LY246492, LY202157, NYX-783, or any salt, solvate, enantiomer, tautomer, stereoisomer, or geometric isomer thereof, or any mixtures thereof.

9. The pharmaceutical composition of claim 1, wherein the N-methyl-D-aspartate receptor modulator comprises ketamine, or any salt, solvate, enantiomer, tautomer, stereoisomer, or geometric isomer thereof, or any mixtures thereof.

10. The pharmaceutical composition of claim 1, wherein the N-methyl-D-aspartate receptor modulator is present in an amount of about 10 mg to about 60 mg, or about 15 mg to about 95 mg, or about 50 mg.

11-12. (canceled)

13. The pharmaceutical composition of claim 1, wherein the μ-opioid receptor modulator is selected from the group consisting of a μ-opioid receptor antagonist, a μ-opioid receptor negative allosteric modulator, and a μ-opioid receptor partial agonist.

14. The pharmaceutical composition of claim 1, wherein the μ-opioid receptor modulator comprises a compound selected from the group consisting of naltrexone, naloxone, nalmefene, nalodeine, or any salt, solvate, enantiomer, tautomer, stereoisomer, or geometric isomer thereof, or any mixtures thereof.

15. The pharmaceutical composition of claim 14, wherein at least one of the following applies:
the naltrexone is formulated for extended-release (long-acting naltrexone) which prolongs the serum half-life of the naltrexone, optionally wherein the long-acting naltrexone is VIVITROL®;
the naloxone is formulated for extended-release (long-acting naloxone) which prolongs the serum half-life of the naloxone;
the nalmefene is formulated for extended-release (long-acting nalmefene) which prolongs the serum half-life of the nalmefene.

16-18. (canceled)

19. The pharmaceutical composition of claim 1, wherein the μ-opioid receptor modulator comprises naltrexone, or any salt, solvate, enantiomer, tautomer, stereoisomer, or geometric isomer thereof, or any mixtures thereof.

20. The pharmaceutical composition of claim 1, wherein the μ-opioid receptor modulator is present in an amount of lower than about 400 mg.

21. The pharmaceutical composition of claim 1, wherein the μ-opioid receptor modulator is present in an amount selected from the group consisting of:
about 300 mg to about 400 mg;
about 200 mg to about 300 mg;
about 100 mg to about 200 mg;
about 5 mg to about 100 mg;
about 25 mg to about 100 mg;
about 1 mg to about 90 mg;
about 2 mg to about 10 mg.

22-27. (canceled)

28. The pharmaceutical composition of claim 1,
wherein the N-methyl-D-aspartate receptor modulator comprises ketamine, or any salt, solvate, enantiomer, tautomer, stereoisomer, or geometric isomer thereof, or any mixtures thereof; and
wherein the μ-opioid receptor modulator comprises naltrexone, or any salt, solvate, enantiomer, tautomer, stereoisomer, or geometric isomer thereof, or any mixtures thereof.

29. The pharmaceutical composition of claim 28,
wherein the N-methyl-D-aspartate receptor modulator is present in an amount of about 10 mg to about 95 mg, and
wherein the μ-opioid receptor modulator is present in an amount of about 10 mg to about 400 mg.

30. A method of treating, ameliorating, or preventing a disease or disorder in a subject in need thereof, the method comprising administering at least one of the following:
(a) the pharmaceutical composition of claim 1 to the subject;
(b) a N-methyl-D-aspartate receptor modulator and a μ-opioid receptor modulator, wherein the μ-opioid receptor modulator is administered in an amount that occupies at least 10% of the subject's μ-opioid receptors in vivo as measured by positron emission tomography with a [¹¹C]-carfentanil ligand.

31. The method of claim 30, wherein the N-methyl-D-aspartate receptor modulator and the μ-opioid receptor modulator are administered concurrently, separately, or sequentially.

32-33. (canceled)

34. The method of claim 30, wherein the N-methyl-D-aspartate receptor modulator is administered once per day, twice per day, three times per day, once per week, twice per week, three times per week, once per month, twice per month, or three times per month.

35-36. (canceled)

37. The method of claim 30, wherein the μ-opioid receptor modulator is administered once per day, twice per day, or three times per day, once per week, twice per week, three times per week, once per month, twice per month, or three times per month.

38-39. (canceled)

40. The method of claim 30, wherein:
the N-methyl-D-aspartate receptor modulator is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous; or
the μ-opioid receptor modulator is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

41. (canceled)

42. The method of claim 30, wherein the disease or disorder is at least one selected from the group consisting of major depressive disorder, major depressive episode in bipolar disorder (bipolar depression), bipolar I disorder, bipolar II disorder, persistent depressive disorder (dysthymia), disruptive mood dysregulation disorder, major depressive disorder (including major depressive episode), premenstrual dysphoric disorder, substance/medication-induced depressive disorder, depressive disorder due to another medical condition, other specified depressive disorder, unspecified depressive disorder, anxiety disorder, generalized anxiety disorder, social anxiety disorder (social phobia), specific phobia, panic disorder, agoraphobia, separation anxiety disorder, selective mutism, substance-induced anxiety disorder, medication-induced anxiety disorder, anxiety disorder due to another medical condition, borderline personality disorder, treatment-resistant depression, unspecified anxiety disorder, and posttraumatic stress disorder.

43. The method of claim 30, wherein the subject suffers from a comorbid substance use disorder.

44. The method of claim 30, wherein administration of the μ-opioid receptor modulator prevents, ameliorates, or minimizes abuse of the N-methyl-D-aspartate receptor modulator and an abused substance that is not the N-methyl-D-aspartate receptor modulator.

45. The method of claim 44, wherein the abused substance is selected from the group consisting of alcohol, a stimulant, an opioid, cannabis, a hallucinogen, an inhalant, a sedative, a hypnotic, an anxiolytic, tobacco, caffeine, nicotine, and other (unknown) substances.

46. The method of claim 45, wherein at least one applies:
the stimulant comprises cocaine or amphetamine;
the hallucinogen comprises lysergic acid diethylamide (LSD) or phencyclidine;
the anxiolytic comprises a barbiturate or a benzodiazepine.

47-48. (canceled)

49. The method of claim 30, wherein the administering has at least one effect selected from the group consisting of reduced anxiety, reduced irritability, reduced anger, and reduced alcohol consumption.

50. A method for treating, ameliorating, or preventing a disease or disorder in a subject in need thereof, the method comprising administering to the subject a coformulation comprising a N-methyl-D-aspartate receptor modulator and a μ-opioid receptor modulator,
wherein the coformulation is administered to the subject repeatedly.

51. The method of claim 50, wherein the N-methyl-D-aspartate receptor modulator is selected from the group consisting of a N-methyl-D-aspartate receptor antagonist, a N-methyl-D-aspartate receptor negative allosteric modulator, and a N-methyl-D-aspartate receptor partial agonist.

52. The method of claim 50, wherein the N-methyl-D-aspartate receptor modulator comprises a compound selected from the group consisting of ketamine, R-ketamine, S-ketamine, nitrous oxide, memantine, amantadine, racemic dextromethorphan, dextromethorphan, lanicemine, phencyclidine, dizocilpine, CERC-301, CGP 37849, 1-aminocylopropanecarboxylic acid, traxoprodil, Ro 25-6981, eliprodil, methoxetamine, CPPene, AP5, AP7, Selfotel (CGS-19755), minocycline, nitromemantine, PD-137889, rolicyclidine, tenocyclidine, methoxydine, tiletamine, neramexane, etoxadrol, dexoxadrol, WMS-2539, NEFA, remacemide, 3-MeO-PCP, 8A-PDHQ, atomoxetine, AZD6765, agmatine, chloroform, delucemine, dextrallorphan, dextrorphan, diphenidine, eticyclidine, gacyclidine, aptiganel, HU-211, huperzine A, dipeptide D-Phe-L-Tyr, ibogaine, rhynchophylline, rapastinel, NRX-1074, 7-Chlorokynurenic acid, 4-Chlorokynurenine, 5,7-Dichlorokynurenic acid, Kynurenic acid, TK-40, L-Phenylalanine, xenon, methadone, EU1180-438, radiprodil, Ifenprodil, TCN-201, MPX-004, MPX-007, NAB-14, EVT-101, QNZ-46, DQP-1105, pregnanolone sulfate (3α5βS), UBP608, UBP618, UBP551, UBP512, HA-966, felbamate, PEAQX (NVP-AAM077), PD0196860, RGH896, MK0657, L701324, LY293558, LY300164, LY246492, LY202157, NYX-783, or any salt, solvate, enantiomer, tautomer, stereoisomer, or geometric isomer thereof, or any mixtures thereof.

53. The method of claim 50, wherein the N-methyl-D-aspartate receptor modulator comprises ketamine, or any salt, solvate, enantiomer, tautomer, stereoisomer, or geometric isomer thereof, or any mixtures thereof.

54. The method of claim 50, wherein the N-methyl-D-aspartate receptor modulator is present in an amount of about 10 mg to about 60 mg, or about 15 mg to about 95 mg, or about 50 mg.

55-56. (canceled)

57. The method of claim 50, wherein the μ-opioid receptor modulator is selected from the group consisting of a μ-opioid receptor antagonist, a μ-opioid receptor negative allosteric modulator, and a μ-opioid receptor partial agonist.

58. The method of claim 50, wherein the μ-opioid receptor modulator comprises a compound selected from the group consisting of naltrexone, naloxone, nalmefene, nalodeine, or any salt, solvate, enantiomer, tautomer, stereoisomer, or geometric isomer thereof, or any mixtures thereof.

59. The method of claim 50, wherein the μ-opioid receptor modulator comprises naltrexone, or any salt, solvate, enantiomer, tautomer, stereoisomer, or geometric isomer thereof, or any mixtures thereof.

60. The method of claim 50, wherein the μ-opioid receptor modulator is present in an amount selected from the group consisting of:
lower than about 400 mg;
about 300 mg to about 400 mg;
about 200 mg to about 300 mg;
about 100 mg to about 200 mg;
about 5 mg to about 100 mg;
about 25 mg to about 100 mg;
about 1 mg to about 90 mg; and
2 mg to about 10 mg.

61-67. (canceled)

68. The method of claim 50, wherein the coformulation is administered once per day, twice per day, three times per day, once per week, twice per week, three times per week, once per month, twice per month, or three times per month.

69-70. (canceled)

71. The method of claim 50, wherein the coformulation is administered to the subject by a route selected from the group consisting of intranasal, inhalational, topical, oral, buccal, rectal, pleural, peritoneal, vaginal, intramuscular, subcutaneous, transdermal, epidural, intratracheal, otic, intraocular, intrathecal, and intravenous.

72. The method of claim 50, wherein the disease or disorder is at least one selected from the group consisting of major depressive disorder, major depressive episode in bipolar disorder (bipolar depression), bipolar I disorder, bipolar II disorder, persistent depressive disorder (dysthymia), disruptive mood dysregulation disorder, major depressive disorder (including major depressive episode), premenstrual dysphoric disorder, substance/medication-induced depressive disorder, depressive disorder due to another medical condition, other specified depressive disorder, unspecified depressive disorder, anxiety disorder, generalized anxiety disorder, social anxiety disorder (social phobia), specific phobia, panic disorder, agoraphobia, separation anxiety disorder, selective mutism, substance-induced anxiety disorder, medication-induced anxiety disorder, anxiety disorder due to another medical condition, borderline personality disorder, treatment-resistant depression, unspecified anxiety disorder, and posttraumatic stress disorder.

73. The method of claim 50, wherein the subject suffers from a comorbid substance use disorder.

74. The method of claim 50, wherein presence of the μ-opioid receptor modulator prevents, ameliorates, or minimizes abuse of the N-methyl-D-aspartate receptor modulator and an abused substance that is not the N-methyl-D-aspartate receptor modulator.

75. The method of claim 74, wherein the abused substance is selected from the group consisting of alcohol, a stimulant, an opioid, cannabis, a hallucinogen, an inhalant, a sedative, a hypnotic, an anxiolytic, tobacco, caffeine, nicotine, and other (unknown) substances.

76. The method of claim 75, wherein at least one of the following applies:
the stimulant comprises cocaine or amphetamine;
the hallucinogen comprises lysergic acid diethylamide (LSD) or phencyclidine;
the anxiolytic comprises a barbiturate or a benzodiazepine.

77-78. (canceled)

79. The method of claim 50, wherein the administering has at least one effect selected from the group consisting of reduced anxiety, reduced irritability, reduced anger, and reduced alcohol consumption.
